(19) United States
(12) Patent Application Publication
(10) Pub. No.: US 2026/0247860 A1
(43) Pub. Date: Aug. 20, 2026

(54) ORGANIC COMPOUNDS AND ORGANIC LIGHT-EMITTING DIODE COMPRISING THE SAME

(71) Applicant: MATERIAL SCIENCE CO., LTD., Gyeonggi-do (KR)

(72) Inventors: Sang Mee KIM, Gyeonggi-do (KR); Tae Wan LEE, Gyeonggi-do (KR); Seung Uk CHO, Gyeonggi-do (KR)

(21) Appl. No.: 19/537,848

(22) Filed: Feb. 12, 2026

(30) Foreign Application Priority Data
Feb. 18, 2025 (KR) 10-2025-0020986
Feb. 12, 2026 (KR) 10-2026-0028979

(51) Int. Cl. H10K 85/60; H10K 50/11

(52) U.S. Cl. CPC H10K 85/6572; H10K 50/11; H10K 85/633

(57) ABSTRACT

Provided are a novel organic compound and an organic light-emitting diode including the same.

The organic light-emitting diode includes a first electrode, a second electrode, and one or more organic material layers disposed between the first electrode and the second electrode, and at least one of the one or more organic material layers includes the organic compound represented by Chemical Formula A of the present invention and thus may improve driving voltage and lifetime characteristics of the organic light-emitting diode.

### CROSS-REFERENCE TO RELATED APPLICATION

This application claims priority to Korean Patent Application No. 10-2025-0020986 filed on Feb. 18, 2025, and No. 10-2026-0028979 filed on Feb. 12, 2026, all the benefits accruing therefrom under 35 U.S.C. § 119, the contents of which are incorporated by reference in their entirety.

### BACKGROUND

The present disclosure herein relates to an organic compound, and an organic light-emitting diode comprising the same.

An organic light-emitting diode (OLED), compared to other flat panel display devices such as typical liquid crystal displays (LCDs), plasma display panels (PDPs), and field emission displays (FEDs), it has a simpler structure and various advantages in a manufacturing process. Due to high luminance, excellent viewing angle characteristics, fast response speed, and low operation voltage thereof, the organic light-emitting diode is being actively developed and commercialized for use as light sources in flat panel displays such as wall-mounted TVs, as well as in backlights for displays, lighting, and billboard.

An organic light-emitting diode is composed of an organic material layer disposed between two electrodes. The organic light-emitting diode is an element utilizing the principle in which electrons and holes are injected into an emitting layer from the two electrodes, respectively, and thus excitons are generated due to combination of the electrons and holes, thereby emitting light when the generated excitons relax from an excited state to the ground state.

The organic light-emitting diode may include at least one emitting layer. Generally, the organic light-emitting diode provided with a plurality of emitting layers includes emitting layers that emit light having different peak wavelengths and thus may implement a predetermined color through combination of light having different peak wavelengths from each other.

Such an organic light-emitting diode may be classified into a top light-emitting element and a bottom light-emitting element structure. The top light-emitting diode emits light generated in the emitting layer toward a translucent or transparent second electrode (cathode) using a reflective first electrode (anode). On the contrary, in the case of a bottom light-emitting element, a reflective second electrode is used to emit light generated in the emitting layer and reflected from the second electrode toward the transparent first electrode which is in the direction of a driving thin-film transistor.

### PRIOR ART DOCUMENTS

### Patent Documents

(Prior Art Document 1) Korean Patent Publication No. 10-2022-0156258 (Prior Art Document 2) Korean Patent Publication No. 10-2024-0044902

### SUMMARY

The present disclosure provides a novel organic compound and an organic light-emitting diode comprising the same.

In addition to the above-described tasks, an embodiment according to the inventive concept may also be used to achieve other tasks that are not specifically described.

The present disclosure is not limited to the tasks described above, and other tasks and effects of the present disclosure that are not described may be understood from the following description and will become clearer through embodiments of the present disclosure.

In addition, it will be easily understood that the aspects and effects of the present disclosure can be achieved by the means and combinations thereof set forth in the claims.

In accordance with an exemplary embodiment of the present invention, provided is an organic compound represented by Chemical Formula A below. A definition of Chemical Formula A below is the same as described in the present specification and claims.

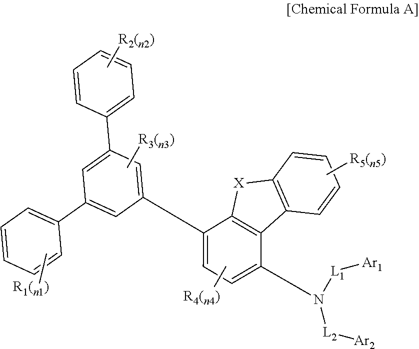

In Chemical Formula A above,
X is oxygen (O), or sulfur(S),
n1 and n2 are each an integer of 0 to 5,
n3 is an integer of 0 to 3,
n4 is an integer of 0 to 2,
n5 is an integer of 0 to 4,
L₁ and L₂ are the same as, or different from each other and are each independently a single bond, or a substituted or unsubstituted arylene group having 6 to 30 carbon atoms,
Ar₁ and Ar₂ are the same as, or different from each other and are each independently a substituted or unsubstituted aryl group having 6 to 30 carbon atoms, or a substituted or unsubstituted heteroaryl group having 3 to 60 carbon atoms,
R₁ to R₅ are the same as, or different from each other and are each independently one selected from the group consisting of a hydrogen atom, a deuterium atom, a substituted or unsubstituted alkyl group having 1 to 10 carbon atoms, a substituted or unsubstituted aryl group having 6 to 30 carbon atoms, and a substituted or unsubstituted heteroaryl group having 3 to 60 carbon atoms, and
when L₁, L₂, Ar₁, Ar₂, and R₁ to R₅ are substituted, the substituent may be one or more selected from the group consisting of a deuterium atom, a cyano group, a nitro group, a halogen group, a hydroxyl group, an alkyl group having 1 to 10 carbon atoms, a cycloalkyl group having 3 to 10 carbon atoms, an aryl group having 6 to 30 carbon atoms, a heteroaryl group having 3 to 60 carbon atoms, an alkylamino group having 1 to 10 carbon atoms, an arylamino group having 6 to 30 carbon atoms, a heteroarylamino group having 1 to 30 carbon atoms, an alkylsilyl group having a 1 to 10 carbon atoms, an arylsilyl group having 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms, and if there are a plurality of substituents, the substituents are the same as or different from each other.

In accordance with another exemplary embodiment of the present invention, an organic light-emitting diode may be provided, which includes a first electrode, a second electrode facing the first electrode, and one or more organic material layers disposed between the first electrode and the second electrode, and at least one among the organic material layers include the organic compound represented by Chemical Formula A above.

The organic compound represented Chemical Formula A of the present invention may achieve excellent hole transport characteristics.

In addition, one or more among the hole transport layer and the hole transport auxiliary layer of the organic light-emitting diode of the present invention includes an organic compound represented by Chemical Formula A, according to the present invention, and thus the organic light-emitting diode may have improved driving voltage, external quantum efficiency, and lifetime characteristics.

In addition, when the organic compound represented by Chemical Formula A according to the present invention is used as a material for the hole transport auxiliary layer, an appropriate energy level for the hole transport auxiliary layer, which serves to transport holes from the hole transport layer to the emitting layer and to block electrons migrating from the emitting layer, may be achieved.

In addition, even when the hole transport layer and/or the hole transport auxiliary layer including the organic compound represented by Chemical Formula A is combined with an emitting layer with any color, the organic light-emitting diode of the present invention may excellently achieve target color coordinates of the emitting layer.

The effects of the present invention are not limited to the above-described effects, and other effects not described will be clearly understood by those skilled in the art from the entire description of the present specification.

### DETAILED DESCRIPTION OF EMBODIMENTS

The above-described objects, features, and advantages will be described in detail below, which will enable a person of ordinary skill in the art to easily practice the technical spirit of the present invention.

In the description of the present specification, specific descriptions of related well-known technologies will be omitted if it is determined that they may unnecessarily obscure the gist of the present invention.

Unless the term “only” is used, the use of terms such as “comprising,” “having”, “consisting of”, “disposed”, or “provided with” in this specification does not preclude the addition of other components. The singular expression of a component is intended to include a plural form as well, unless the context clearly indicates otherwise.

In interpreting a component in the present specification, it is construed to include an error range even if there is no additional explicit description.

In the present specification, the expression that a certain component is disposed “on (or under)” or “above (or below)” another component means not only that another component is disposed in contact with a top (or bottom) surface of the component, but also that a third component may be disposed between the component and the certain component disposed on (or under) the component.

In this specification, the expressions “at least one among a, b, and c” and “among a, b, or c” are intended to encompass all of “a individually”, “b individually”, and “c individually”, “a and b”, “a and c”, “b and c”, or “all a, b, and c”.

The term “halogen group” used in this specification, includes fluorine, chlorine, bromine, and iodine.

The term “alkyl group” used in this specification indicates both a linear-chain alkyl radical, and a branched-chain alkyl radical. Unless otherwise specified, the alkyl group contains 1 to 30 carbon atoms and may include methyl, ethyl, propyl, isopropyl, butyl, sec-butyl, isobutyl, tert-butyl, pentyl, isoamyl, and hexyl, but an embodiment of the present invention is not limited thereto. Additionally, the alkyl group may be optionally substituted.

The term “cycloalkyl group” used in this specification indicates a cyclic alkyl radical. Unless otherwise specified, the cycloalkyl group contains 3 to 20 carbon atoms and may include cyclopropyl, cyclobutyl, cyclopentyl, cyclohexyl, norbornyl, adamantyl, etc., but an embodiment of the present invention is not limited thereto. Additionally, the cycloalkyl group may be optionally substituted.

Unless otherwise specified, the term “alkenyl group” used in this specification indicates both a linear-chain alkenyl radical and a branched-chain alkenyl radical, having one or more carbon-carbon double bonds. Unless otherwise specified, the alkenyl group contains 2 to 30 carbon atoms and may include vinyl, allyl, isopropenyl, 2-butenyl, etc., but an embodiment of the present invention is not limited thereto. Additionally, the alkenyl group may be optionally substituted.

The term “cycloalkenyl group” used in this specification indicates a cyclic alkenyl radical. Unless otherwise specified, the cycloalkenyl group contains 3 to 20 carbon atoms and may be optionally substituted.

The term “alkynyl group” used in this specification indicates both a linear-chain alkynyl radical and a branched-chain alkynyl radical, having one or more carbon-carbon triple bonds. Unless otherwise specified, the alkynyl group contains 2 to 30 carbon atoms and may include ethynyl, 2-propynyl, etc., but an embodiment of the present invention is not limited thereto. Additionally, the alkynyl group may be optionally substituted.

The term “cycloalkynyl group” used in this specification indicates a cyclic alkynyl radical. Unless otherwise specified, the cycloalkynyl group contains 3 to 20 carbon atoms and may be optionally substituted.

The terms “aralkyl group” and “arylalkyl group” used in this specification are used interchangeably used and indicate an alkyl group having an aromatic group as a substituent, and additionally, the aralkyl group (arylalkyl group) may be optionally substituted.

The terms “aryl group” and “aromatic group” used in this specification have the same meaning, and the aryl group includes both monocyclic and polycyclic ring groups. A polycyclic ring may include a “fused ring” having two or more rings, in which two adjacent rings share two carbon atoms. In addition, a form in which two or more rings are simply attached to each other or fused together may be also included. Unless otherwise specified, the aryl group contains 6 to 30 carbon atoms and may include phenyl, biphenyl, terphenyl, naphthyl, phenanthrenyl, anthracenyl, fluorenyl, dimethylfluorenyl, diphenylfluorenyl, spirobifluorenyl, etc., but an embodiment of the present invention is not limited thereto. In addition, the aryl group may be optionally substituted.

The terms “heteroaryl group” and “heteroaromatic group” used in this specification have the same meaning, and the heteroaryl group includes both monocyclic and polycyclic ring groups. A polycyclic ring may include a “fused ring” having two or more rings, in which two adjacent rings share two atoms selected from carbon, and heteroatoms. In addition, a form in which two or more rings are simply attached to each other or fused together may be also included. Unless otherwise specified, the heteroaryl group contains 3 to 60 carbon atoms, and in this case, one or more carbon atoms in the ring is substituted with a heteroatom such as oxygen (O), nitrogen (N), sulfur(S), or selenium (Se), and the heteroaryl group may include: a 6-membered monocyclic ring such as pyridyl, pyrazinyl, pyrimidinyl, pyridazinyl, and triazinyl; a polycyclic ring such as phenoxathiinyl, indolizinyl, indolyl, purinyl, quinolyl, isoquinolyl, benzoxazolyl, benzothiazolyl, benzoimidazolyl, benzofuranyl, dibenzofuranyl, benzothiophenyl, dibenzothiophenyl, carbazolyl, phenylcarbazoly, and 9-phenylcarbazolyl; 2-furanyl; N-imidazolyl; 2-isoxazolyl; 2-pyridinyl; 2-pyrimidinyl; and the like, but an embodiment of the present invention is not limited thereto. In addition, the heteroaryl group may be optionally substituted.

The term “heterocyclic group” used in this specification indicates a group in which at least one of carbon atoms forming an aryl group, a cycloalkyl group, a cycloalkenyl group, a cycloalkynyl group, an arylalkyl group, an arylamino group, or the like is replaced with a heteroatom such as oxygen (O), nitrogen (N), or sulfur(S). Based on this definition, the heterocyclic group may include a heteroaryl group, a heterocycloalkyl group, a heterocycloalkenyl group, a heterocycloalkynyl group, a heteroarylalkyl group, a heteroarylamino group, and the like, and the heterocycle may be optionally substituted.

The term “carbon ring” used in this specification may be used as a term encompassing both an alicyclic ring group such as a “cycloalkyl group”, a “cycloalkenyl group”, and a “cycloalkynyl group”, and an aromatic ring group such as an “aryl group (aromatic ring)”, unless otherwise specifically limited.

The terms “heteroalkyl group”, “heteroalkenyl group”, “heteroalkynyl group”, and “heteroarylalkyl group” used in this specification mean that at least one of carbon atoms forming each group is replaced with a heteroatom such as oxygen (O), nitrogen (N), or sulfur(S), and furthermore, the heteroalkyl group, the heteroalkenyl group, the heteroalkynyl group, and the heteroarylalkyl group may be optionally substituted.

The terms “alkylamino group”, “arylalkylamino group”, “arylamino group”, and “heteroarylamino group” used in this specification indicate groups in which amino groups (or amine groups) are substituted with the above-described alkyl, arylalkyl, aryl, and heteroaryl groups, respectively, and are intended to include all of primary, secondary, and tertiary amino groups (or amine groups). Additionally, the alkylamino group, the arylalkylamino group, the arylamino group, and the heteroarylamino group may be optionally substituted.

The terms “alkylsilyl group”, “arylsilyl group”, “alkoxy group”, “aryloxy group”, “alkylthio group”, “arylthio group” used in this specification indicate that a silyl group, oxy group, and thio group are substituted with each of the alkyl group, and the aryl group, and additionally, the alkylsilyl group, the arylsilyl group, the alkoxy group, the aryloxy group, the alkylthio group, and the arylthio group may be optionally substituted.

The terms “arylene group”, “arylalkylene group”, “heteroarylene group”, “heteroarylalkylene group” used in this specification indicate divalent substituents corresponding to the aryl group, arylalkyl group, heteroaryl group, and heteroarylalkyl group, respectively, each of which contains one more point of substitution. In addition, the arylene group, the arylalkylene group, the heteroarylene group, and the heteroarylalkylene group may be optionally substituted.

The term “substituted” used in this specification means that a hydrogen (H) atom bonded to a carbon, nitrogen, or other atom of the compound of the present invention is replaced with a substituent other than hydrogen. When substituents are in plurality, the substituents may be the same as or different from each other.

The substituent may be each independently selected from deuterium, a cyano group, a trifluoromethyl group, a nitro group, a halogen group, a hydroxyl group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 3 to 60 carbon atoms, a heteroarylalkyl group having 4 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 3 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms.

Each entity and substituent defined in this specification may be the same or different, unless otherwise specified.

Unless otherwise specified, all units used herein are weight (wt)-based. For example, a value expressed in ‘%’ refers to percent by weight (wt %).

Hereinafter, an organic compound according to the present invention and an organic light-emitting diode including the same will be described in detail.

The organic compound of the present invention may be represented by Chemical Formula A below.

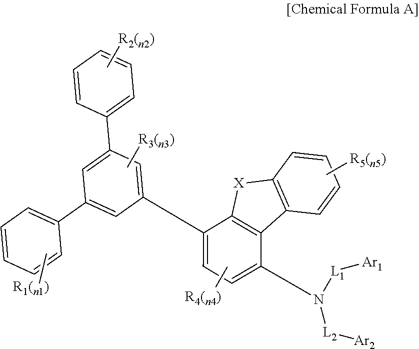

In Chemical Formula A above,
X is selected from oxygen (O), or sulfur(S),
n1 and n2 are each an integer of 0 to 5,
n3 is an integer of 0 to 3,
n4 is an integer of 0 to 2,
n5 is an integer of 0 to 4,
L₁ and L₂ are the same as, or different from each other and are each independently a single bond, or a substituted or unsubstituted arylene group having 6 to 30 carbon atoms,
Ar₁ and Ar₂ are the same as, or different from each other and are each independently a substituted or unsubstituted aryl group having 6 to 30 carbon atoms, or a substituted or unsubstituted heteroaryl group having 3 to 60 carbon atoms,
R₁ to R₅ are the same as, or different from each other and are each independently one selected from the group consisting of hydrogen, deuterium, a substituted or unsubstituted alkyl group having 1 to 10 carbon atoms, a substituted or unsubstituted aryl group having 6 to 30 carbon atoms, or a substituted or unsubstituted heteroaryl group having 3 to 60 carbon atoms,
when L₁, L₂, Ar₁, Ar₂, and R₁ to R₅ are each substituted, the substituent may be one selected from the group consisting of deuterium, a cyano group, a nitro group, a halogen group, a hydroxyl group, an alkyl group having 1 to 10 carbon atoms, a cycloalkyl group having 3 to 10 carbon atoms, an aryl group having 6 to 30 carbon atoms, a heteroaryl group having 3 to 30 carbon atoms, an alkylamino group having 1 to 30 carbon atoms, an arylamino group having 6 to 30 carbon atoms, a heteroarylamino group having a 1 to 30 carbon atoms, an alkylsilyl group having a 1 to 10 carbon atoms, an arylsilyl group having a 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms, and if there are a plurality of substituents, the substituents are the same as or different from each other.

According to an embodiment of the present invention, Ar₁ may be a substituted or unsubstituted aryl group having carbon atoms of 6 to 30, 6 to 25, 6 to 15, 6 to 12, or 6 to 10, or a substituted or unsubstituted heteroaryl group having carbon atoms of 3 to 60, 3 to 30, 3 to 18, 3 to 12, 3 to 9, 3 to 8, 3 to 7, 3 to 5, or 3 to 4. For example, Ar₁ may be a substituted or unsubstituted naphthyl group, a substituted or unsubstituted phenanthrenyl group, a substituted or unsubstituted terphenyl group, a substituted or unsubstituted dibenzofuranyl group, a substituted or unsubstituted dibenzothiophenyl group, a substituted or unsubstituted phenyl-naphthyl group, a substituted or unsubstituted phenyl-phenanthrenyl group, a substituted or unsubstituted phenyl-dibenzofuranyl group, or a substituted or unsubstituted phenyl-dibenzothiophenyl group.

According to an embodiment of the present invention, Ar₁ may be a substituted or unsubstituted aryl group having carbon atoms of 6 to 30, 6 to 25, 6 to 15, 6 to 12, or 6 to 10, or a substituted or unsubstituted heteroaryl group having carbon atoms of 3 to 60, 3 to 30, 3 to 18, 3 to 12, 3 to 9, 3 to 8, 3 to 7, 3 to 5, or 3 to 4. For example, Ar₁ may be a substituted or unsubstituted naphthyl group, a substituted or unsubstituted phenanthrenyl group, a substituted or unsubstituted dibenzofuranyl group, a substituted or unsubstituted dibenzothiophenyl group, a substituted or unsubstituted phenyl-naphthyl group, a substituted or unsubstituted phenyl-phenanthrenyl group, a substituted or unsubstituted phenyl-dibenzofuranyl group, or a substituted or unsubstituted phenyl-dibenzothiophenyl group.

According to an embodiment of the present invention, Ar₂ may be a substituted or unsubstituted aryl group having carbon atoms of 6 to 30, 6 to 25, 6 to 15, 6 to 12, or 6 to 10, or a substituted or unsubstituted heteroaryl group having carbon atoms of 3 to 60, 3 to 30, 3 to 18, 3 to 12, 3 to 9, 3 to 8, 3 to 7, 3 to 5, or 3 to 4. For example, Ar₂ may be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted dimethylfluorene group, a substituted or unsubstituted diphenylfluorene group, a substituted or unsubstituted spirofluorene group, a substituted or unsubstituted dibenzofuranyl group, a substituted or unsubstituted dibenzothiophenyl group, a substituted or unsubstituted phenyl-naphthyl group, a substituted or unsubstituted phenyl-phenanthrenyl group, a substituted or unsubstituted phenyl-dibenzofuranyl group, or a substituted or unsubstituted phenyl-dibenzothiophenyl group.

According to an embodiment of the present invention, Ar₂ may be a substituted or unsubstituted aryl group having carbon atoms of 6 to 30, 6 to 25, 6 to 15, 6 to 12, or 6 to 10, or a substituted or unsubstituted heteroaryl group having carbon atoms of 3 to 60, 3 to 30, 3 to 18, 3 to 12, 3 to 9, 3 to 8, 3 to 7, 3 to 5, or 3 to 4. For example, Ar₂ may be a substituted or unsubstituted phenyl group, a substituted or unsubstituted biphenyl group, a substituted or unsubstituted naphthyl group, a substituted or unsubstituted phenanthrenyl group, a substituted or unsubstituted dibenzofuranyl group, a substituted or unsubstituted dibenzothiophenyl group, a substituted or unsubstituted phenyl-naphthyl group, a substituted or unsubstituted phenyl-phenanthrenyl group, a substituted or unsubstituted phenyl-dibenzofuranyl group, a substituted or unsubstituted phenyl-dibenzothiophenyl group.

According to an embodiment of the present invention, L₁ and L₂ may be a single bond (direct linkage) or one among substituents represented by F1 to F3 below. In F1 to F3 below, * represents a bonding position.

According to an embodiment of the present invention, Dn of F1 to F3 below indicates a number of deuterium substituted in a structure, and n indicating the number of deuterium is an integer of 0 or greater.

According to an embodiment of the present invention, n of each of F1 to F3 below is an integer selected from 0 to 4.

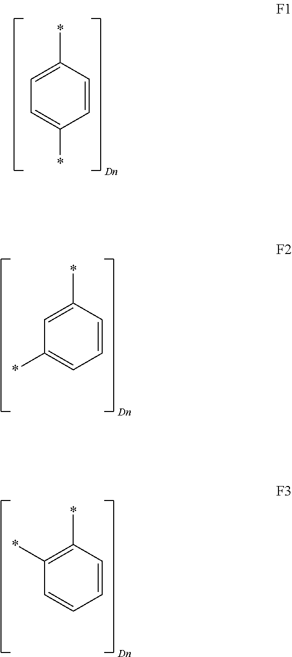

According to an embodiment of the present invention, Ar₁ may be one among substituents represented by M1 to M48 below. In M1 to M48 below, * represents a bonding position.

According to an embodiment of the present invention, Dn of M1 to M48 below indicates a number of deuterium substituted in a structure, and n indicating the number of deuterium is an integer of 0 or greater.

According to an embodiment of the present invention, n of each of M1 and M2 is an integer selected from 0 to 7, n is an integer of 0 to 9 in M3, n is an integer selected from 0 to 7 in M4 to M11, n is an integer selected from 0 to 11 in M12 and M17, n is an integer selected from 0 to 13 in M18 to M20, n is an integer selected from 0 to 11 in M21 to M44, and n is an integer of 0 to 7 in M45 to M48.

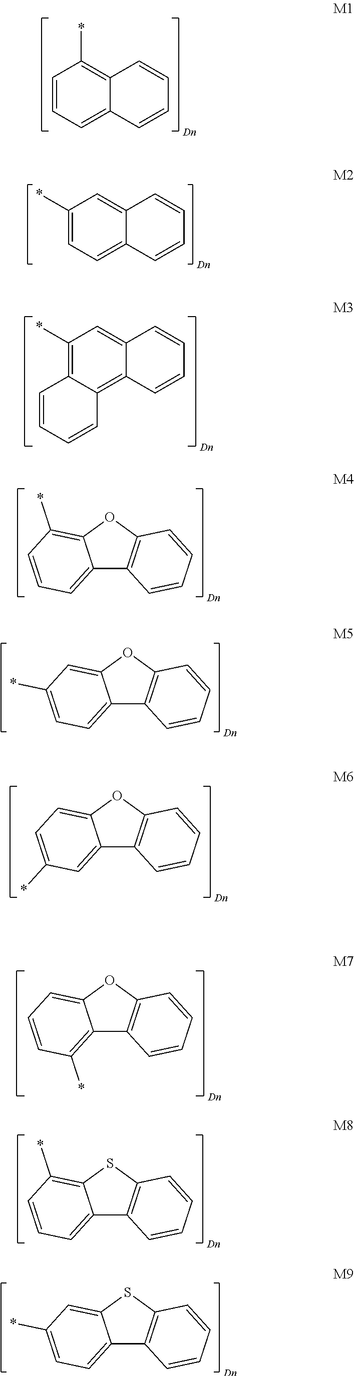

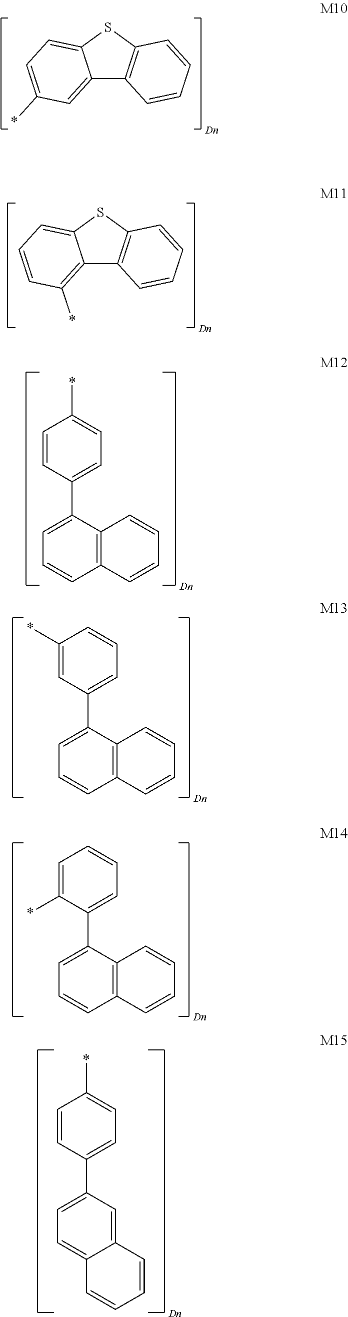

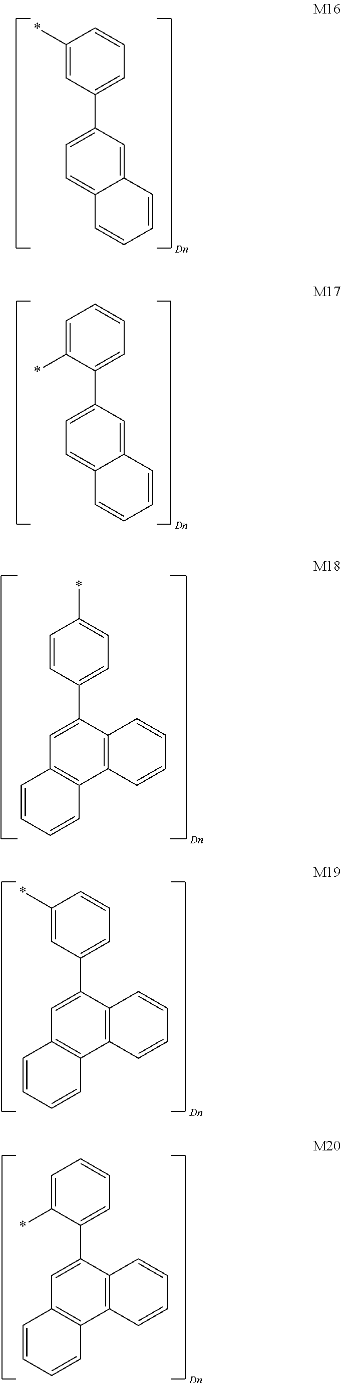

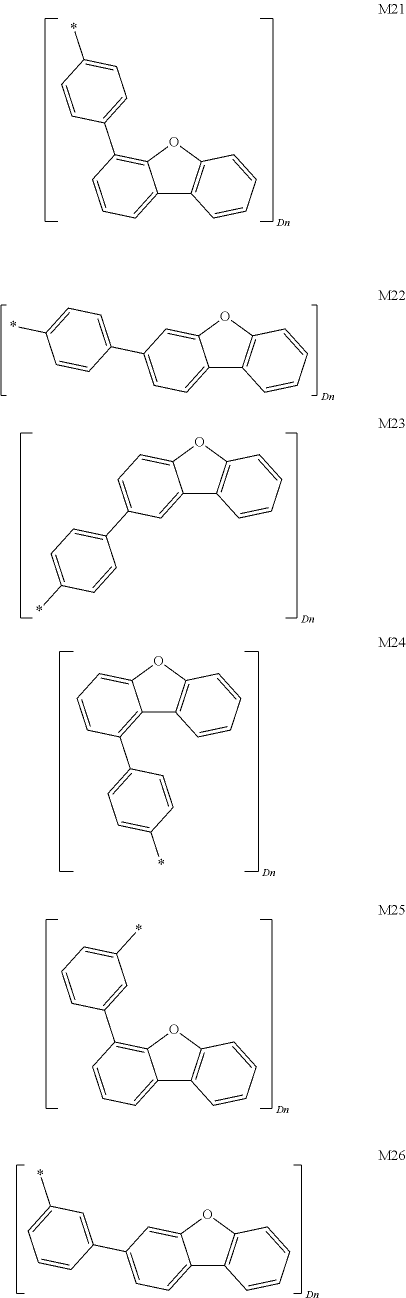

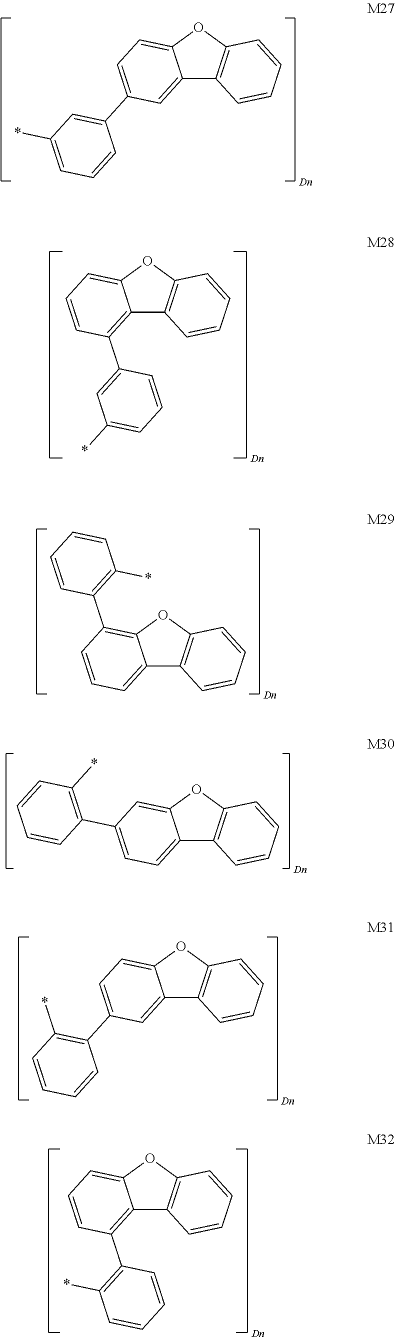

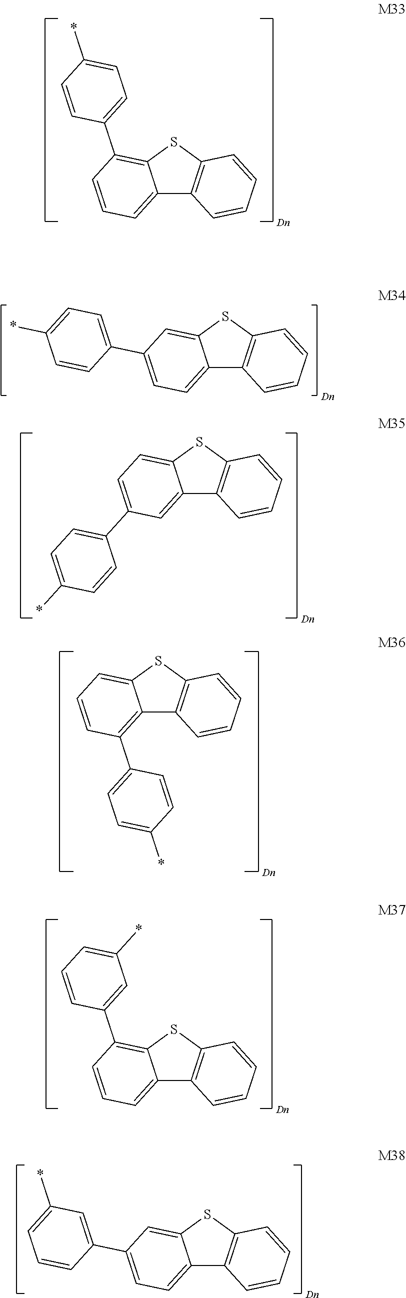

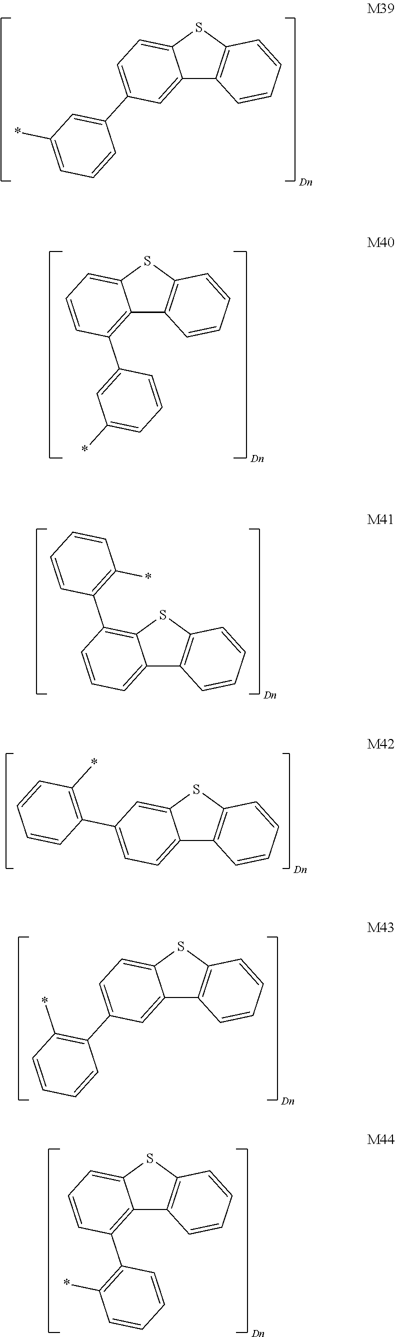

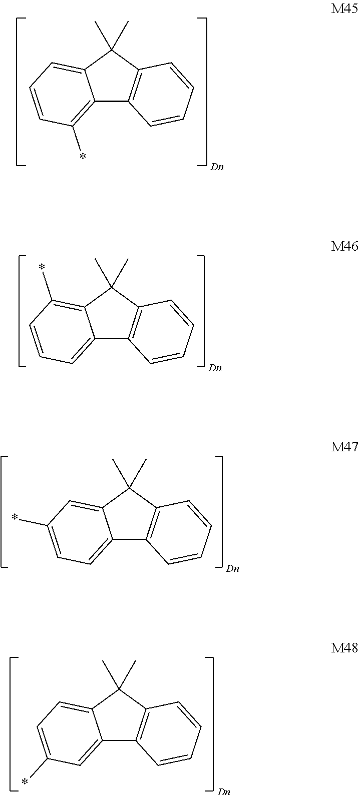

According to an embodiment of the present invention, Ar₂ may be one among substituents represented by A1 to A58 below. In A1 to A58 below, * represents a bonding position.

According to an embodiment of the present invention, Dn of A1 to A58 below indicates a number of deuterium substituted in a structure, and n indicating the number of deuterium is an integer of 0 or greater.

According to an embodiment of the present invention, n of A1 is an integer selected from 0 to 5, n of A2 to A4 is an integer selected from 0 to 9, n of A5 to A10 is an integer selected from 0 to 13, n of A11 and A12 is an integer selected from 0 to 7, n of A13 is an integer selected from 0 to 9, n of A14 to A25 is an integer selected from 0 to 7, n of A26 to A31 is an integer selected from 0 to 11, n of A32 to A34 is an integer selected from 0 to 13, and n of A35 to A58 is an integer selected from 0 to 11.

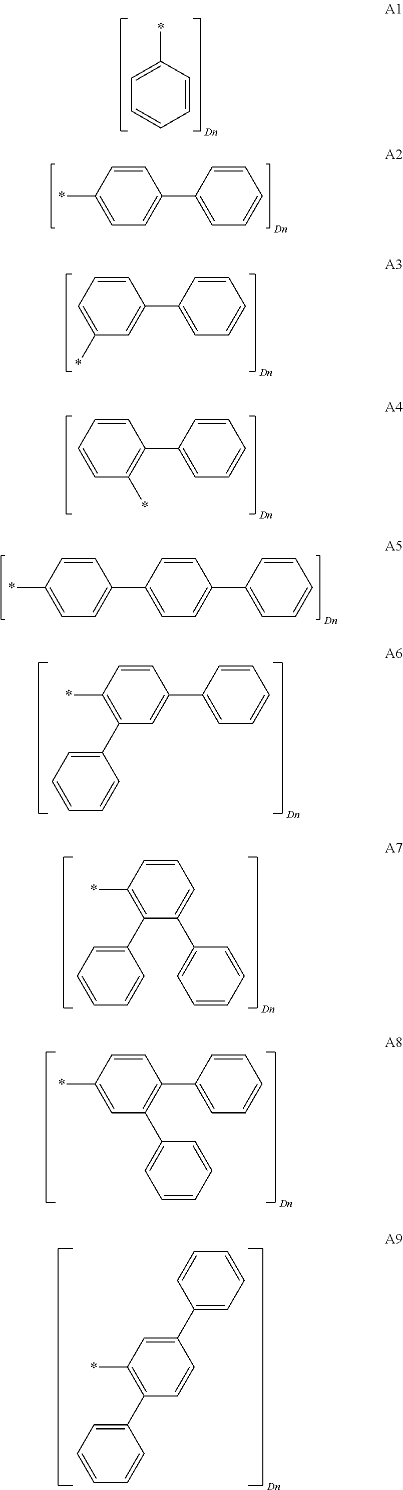

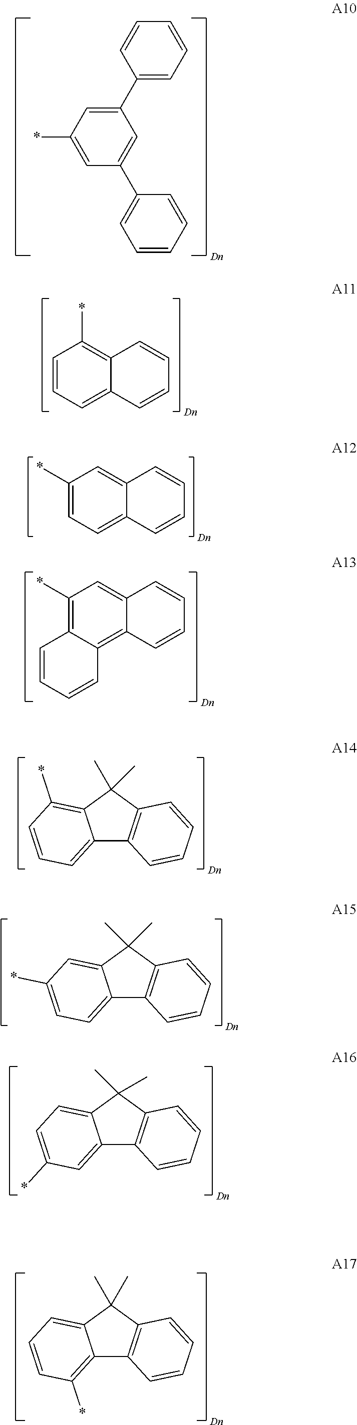

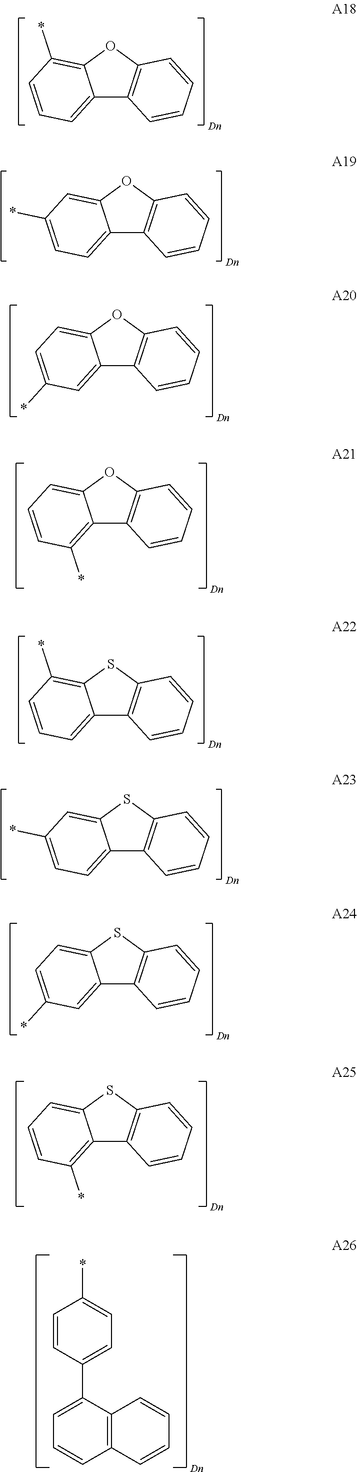

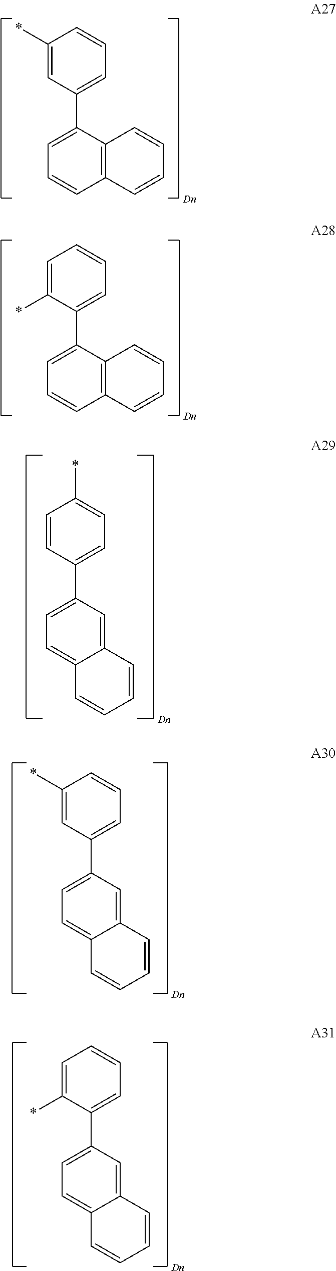

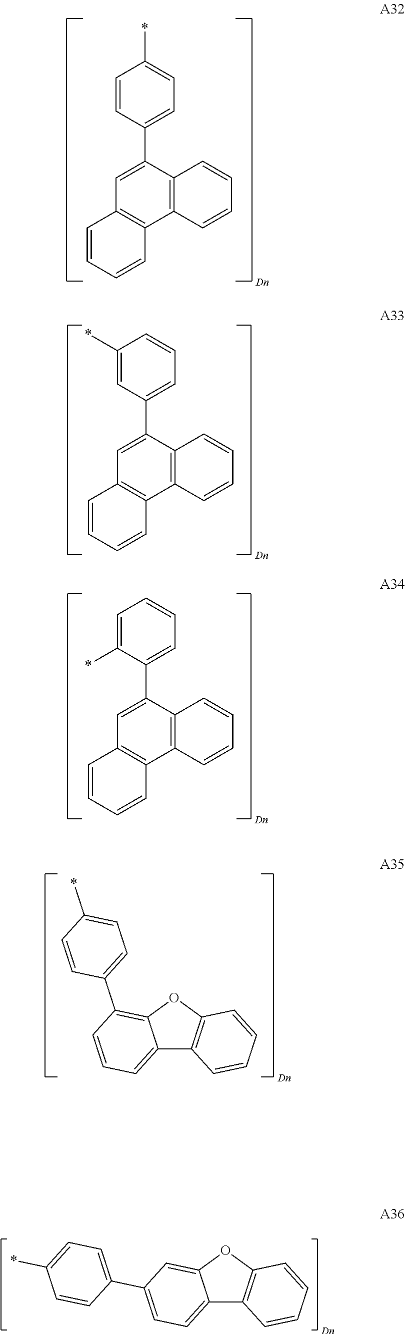

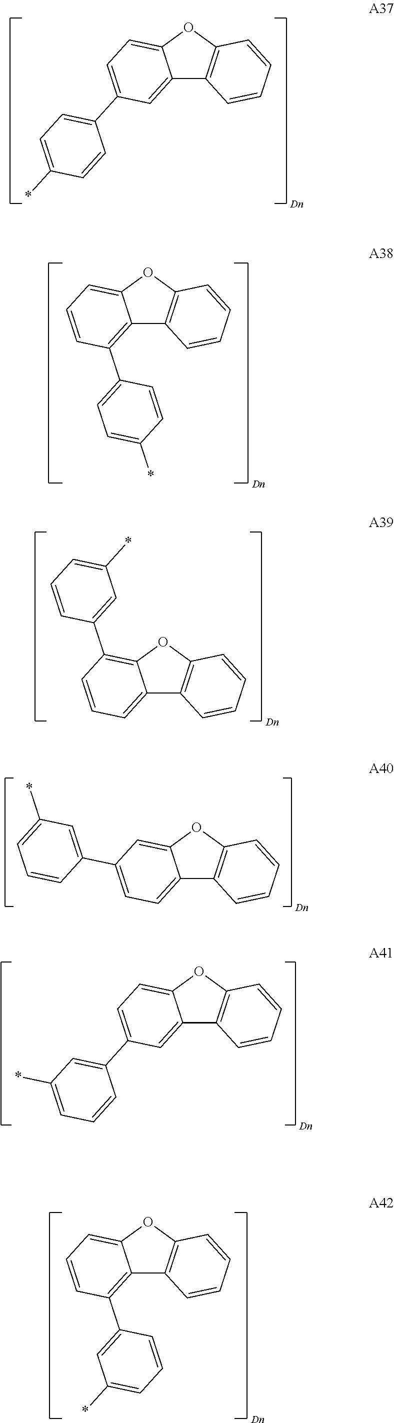

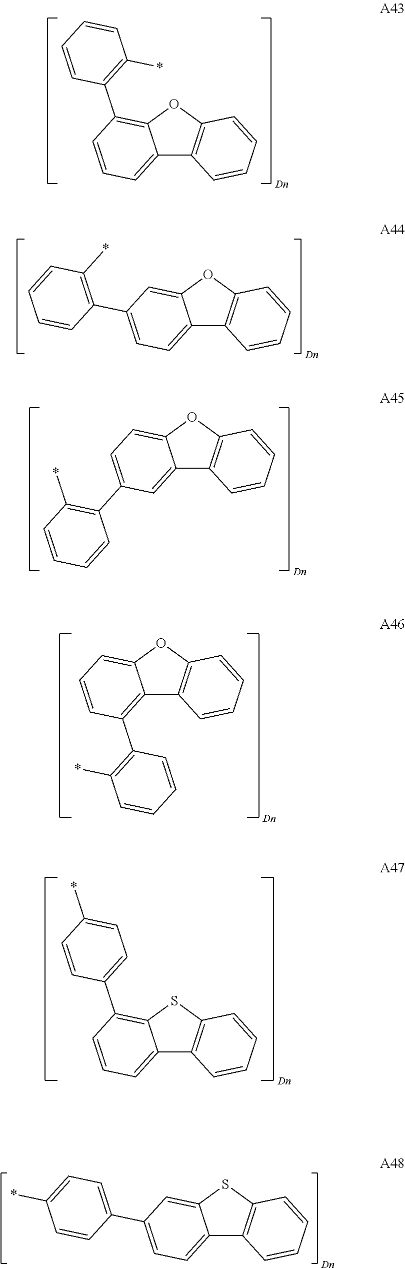

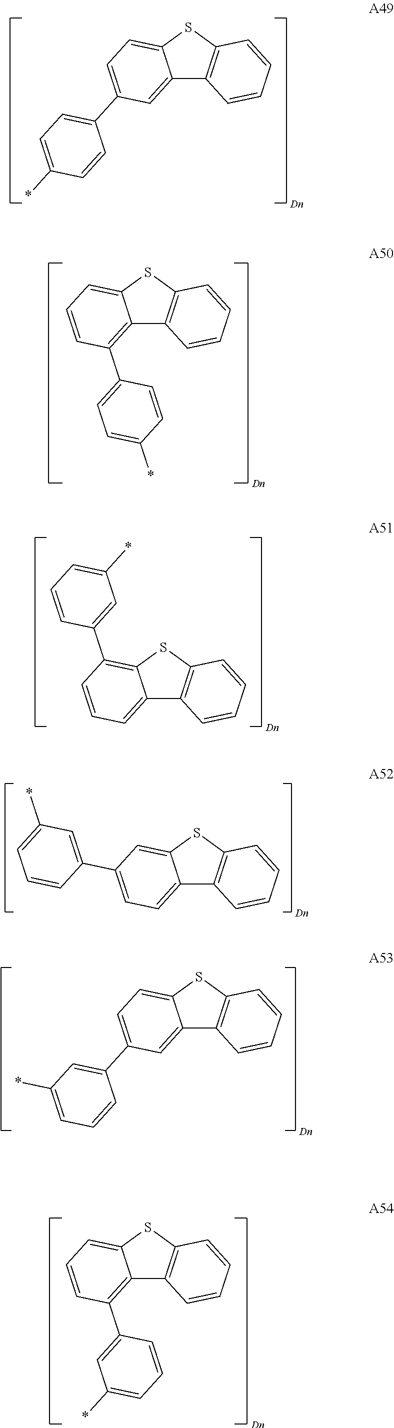

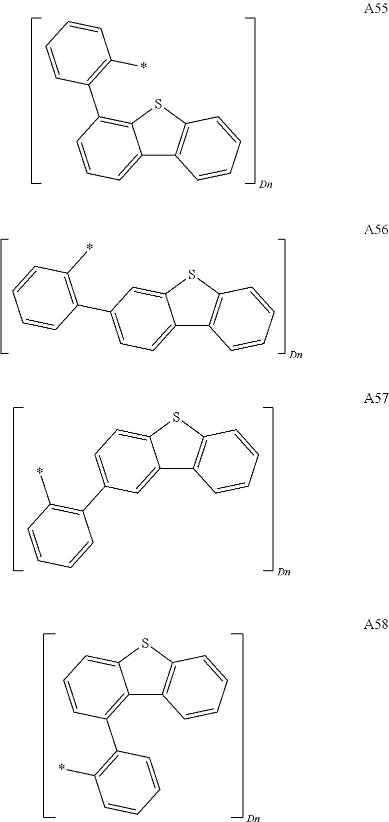

According to an embodiment of the present invention, R₁ to R₅ may be the same as or different from each other, and may be each differently hydrogen, or deuterium.

According to an embodiment of the present invention, the organic compound represented by Chemical Formula A may be selected from the group consisting of the following compounds but is not limited thereto.

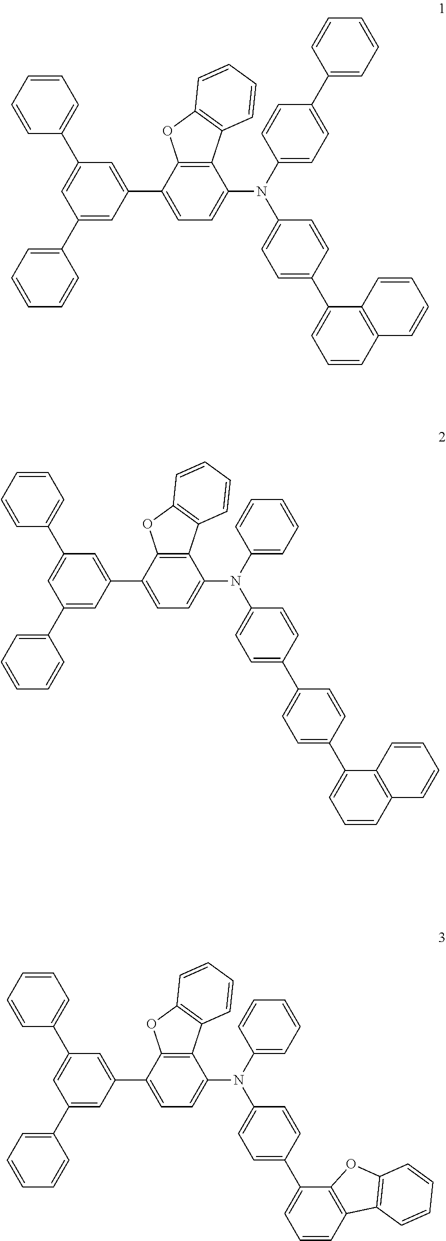 [SMILES: c1ccc(-c2ccc(N(c3ccc(-c4cccc5ccccc45)cc3)c3ccc(-c4cc(-c5ccccc5)cc(-c5ccccc5)c4)c4oc5ccccc5c34)cc2)cc1 ; c1ccc(-c2cc(-c3ccccc3)cc(-c3ccc(N(c4ccccc4)c4ccc(-c5ccc(-c6cccc7ccccc67)cc5)cc4)c4c3oc3ccccc34)c2)cc1 ; c1ccc(-c2cc(-c3ccccc3)cc(-c3ccc(N(c4ccccc4)c4ccc(-c5cccc6c5oc5ccccc56)cc4)c4c3oc3ccccc34)c2)cc1]

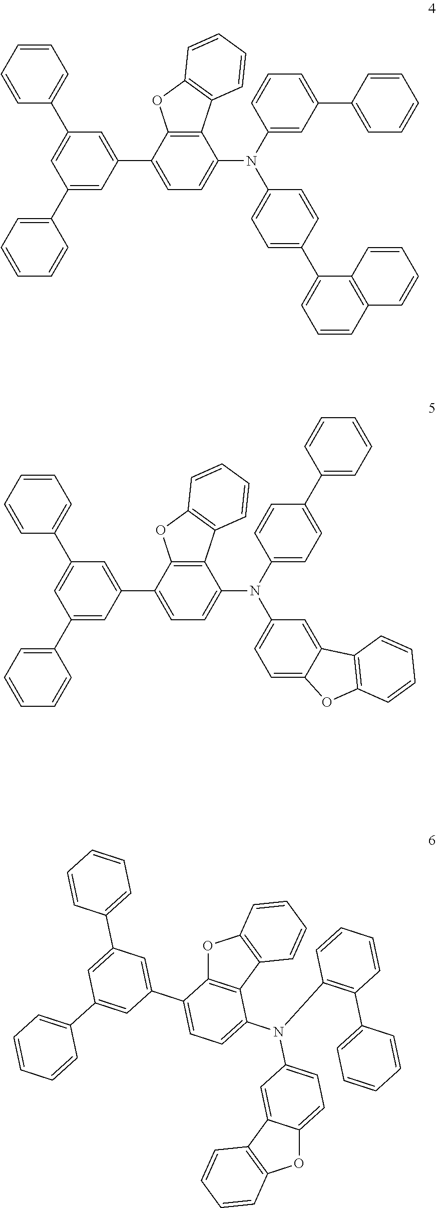 [SMILES: c1ccc(-c2cc(-c3ccccc3)cc(-c3ccc(N(c4ccc(-c5cccc6ccccc56)cc4)c4cccc(-c5ccccc5)c4)c4c3oc3ccccc34)c2)cc1 ; c1ccc(-c2ccc(N(c3ccc4oc5ccccc5c4c3)c3ccc(-c4cc(-c5ccccc5)cc(-c5ccccc5)c4)c4oc5ccccc5c34)cc2)cc1 ; c1ccc(-c2cc(-c3ccccc3)cc(-c3ccc(N(c4ccc5oc6ccccc6c5c4)c4ccccc4-c4ccccc4)c4c3oc3ccccc34)c2)cc1]

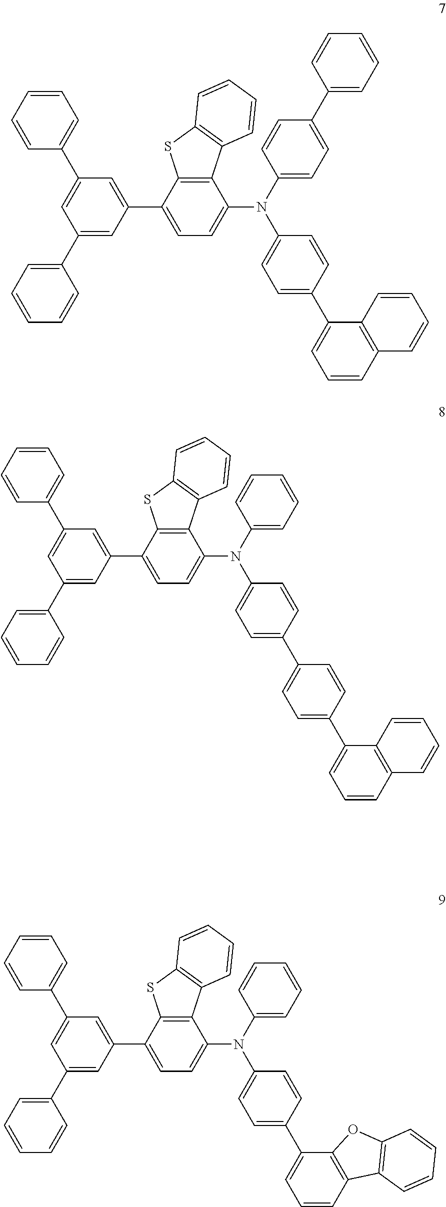 [SMILES: c1ccc(-c2ccc(N(c3ccc(-c4cccc5ccccc45)cc3)c3ccc(-c4cc(-c5ccccc5)cc(-c5ccccc5)c4)c4sc5ccccc5c34)cc2)cc1 ; c1ccc(-c2cc(-c3ccccc3)cc(-c3ccc(N(c4ccccc4)c4ccc(-c5ccc(-c6cccc7ccccc67)cc5)cc4)c4c3sc3ccccc34)c2)cc1 ; c1ccc(-c2cc(-c3ccccc3)cc(-c3ccc(N(c4ccccc4)c4ccc(-c5cccc6c5oc5ccccc56)cc4)c4c3sc3ccccc34)c2)cc1]

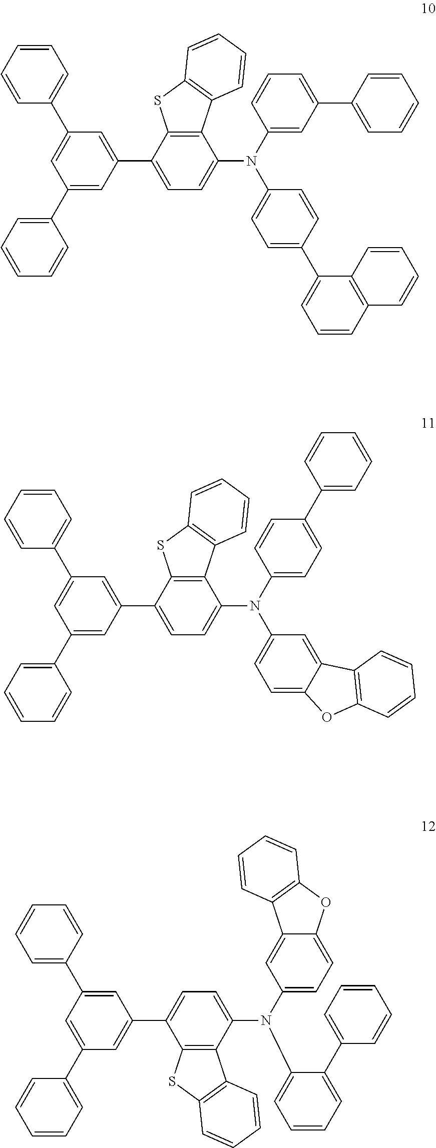 [SMILES: c1ccc(-c2cc(-c3ccccc3)cc(-c3ccc(N(c4ccc(-c5cccc6ccccc56)cc4)c4cccc(-c5ccccc5)c4)c4c3sc3ccccc34)c2)cc1 ; c1ccc(-c2ccc(N(c3ccc4oc5ccccc5c4c3)c3ccc(-c4cc(-c5ccccc5)cc(-c5ccccc5)c4)c4sc5ccccc5c34)cc2)cc1 ; c1ccc(-c2cc(-c3ccccc3)cc(-c3ccc(N(c4ccc5oc6ccccc6c5c4)c4ccccc4-c4ccccc4)c4c3sc3ccccc34)c2)cc1]

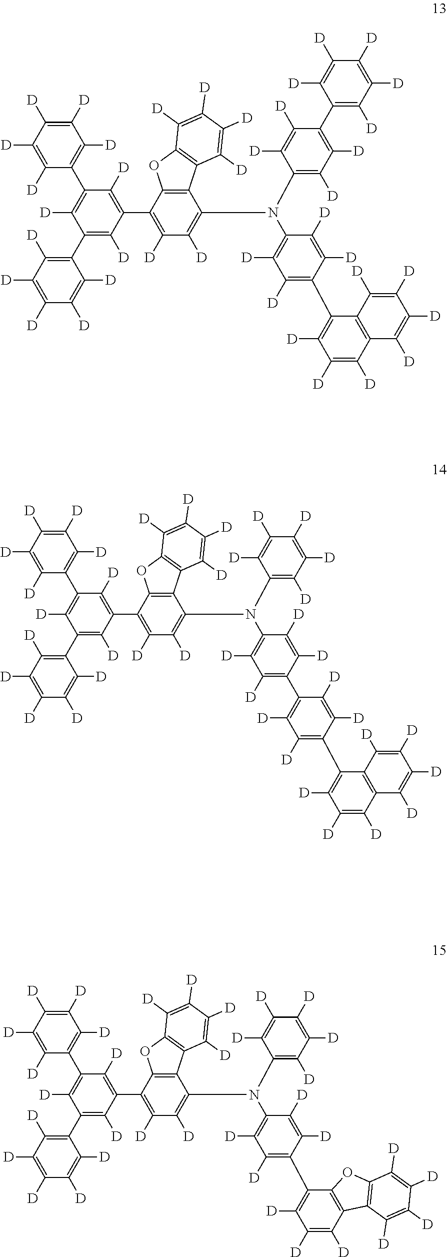

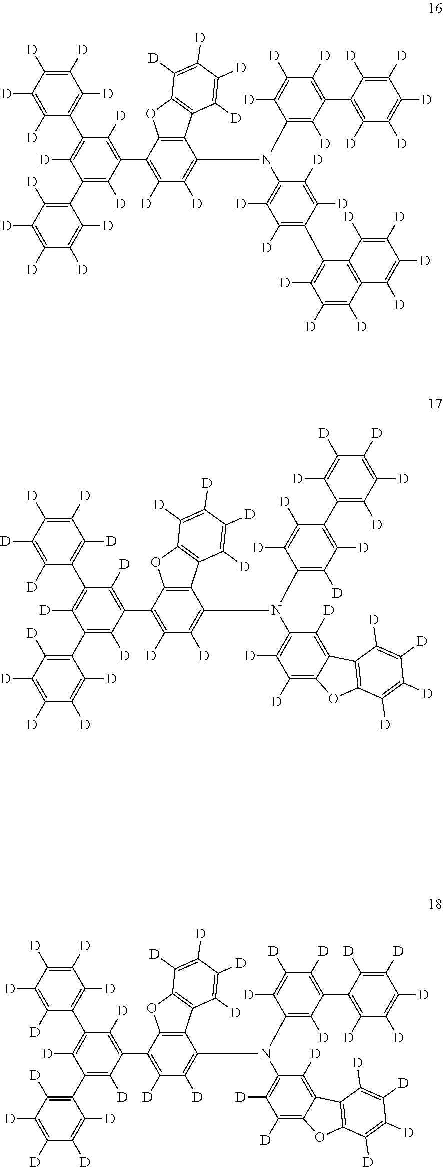

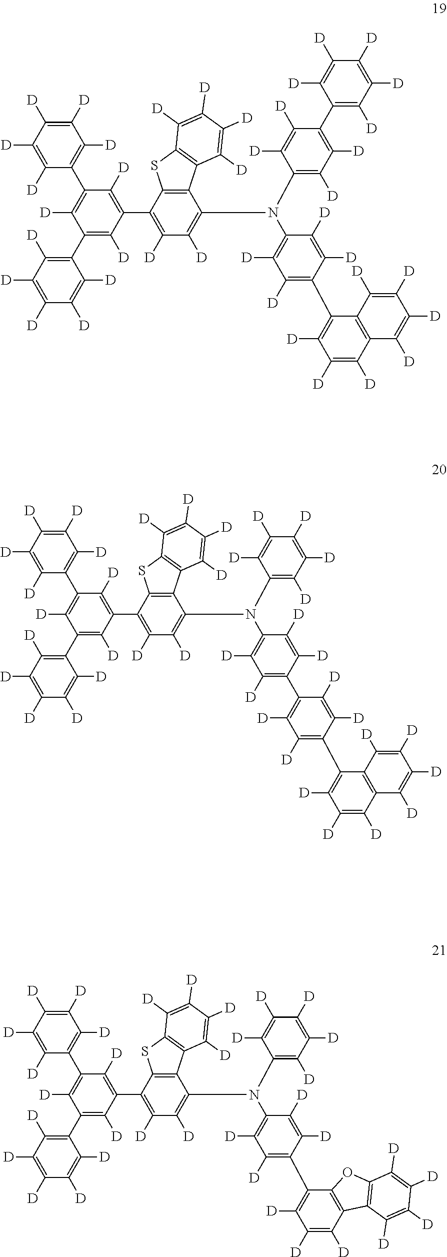

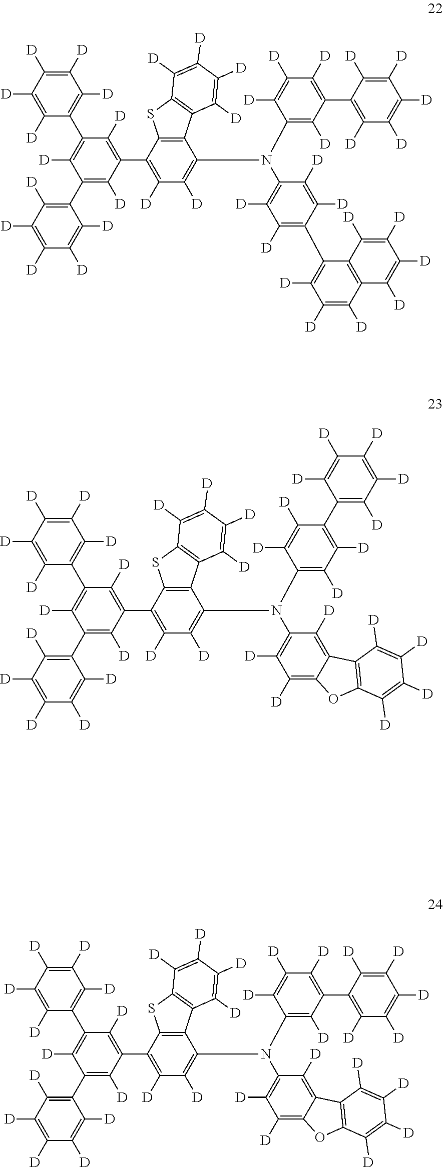

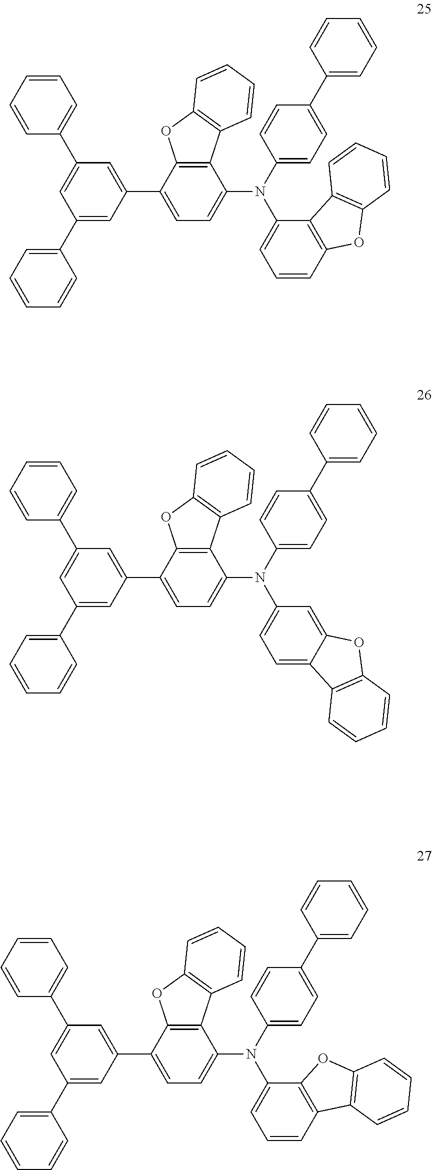 [SMILES: c1ccc(-c2ccc(N(c3cccc4oc5ccccc5c34)c3ccc(-c4cc(-c5ccccc5)cc(-c5ccccc5)c4)c4oc5ccccc5c34)cc2)cc1 ; c1ccc(-c2ccc(N(c3ccc4c(c3)oc3ccccc34)c3ccc(-c4cc(-c5ccccc5)cc(-c5ccccc5)c4)c4oc5ccccc5c34)cc2)cc1 ; c1ccc(-c2ccc(N(c3cccc4c3oc3ccccc34)c3ccc(-c4cc(-c5ccccc5)cc(-c5ccccc5)c4)c4oc5ccccc5c34)cc2)cc1]

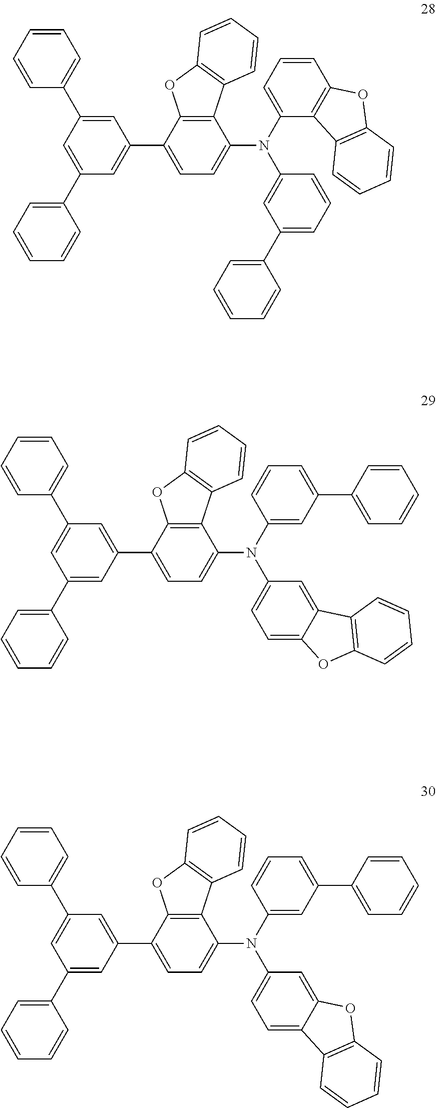 [SMILES: c1ccc(-c2cc(-c3ccccc3)cc(-c3ccc(N(c4cccc(-c5ccccc5)c4)c4cccc5oc6ccccc6c45)c4c3oc3ccccc34)c2)cc1 ; c1ccc(-c2cc(-c3ccccc3)cc(-c3ccc(N(c4cccc(-c5ccccc5)c4)c4ccc5oc6ccccc6c5c4)c4c3oc3ccccc34)c2)cc1 ; c1ccc(-c2cc(-c3ccccc3)cc(-c3ccc(N(c4cccc(-c5ccccc5)c4)c4ccc5c(c4)oc4ccccc45)c4c3oc3ccccc34)c2)cc1]

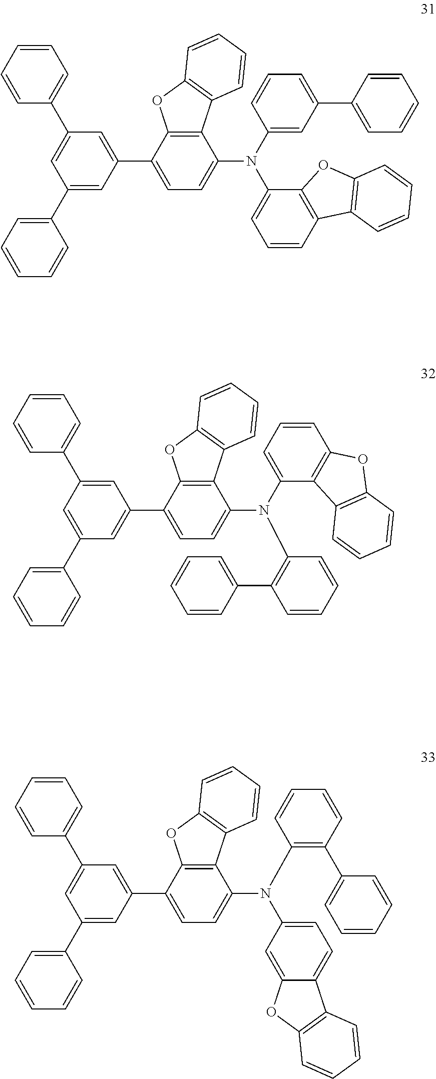 [SMILES: c1ccc(-c2cc(-c3ccccc3)cc(-c3ccc(N(c4cccc(-c5ccccc5)c4)c4cccc5c4oc4ccccc45)c4c3oc3ccccc34)c2)cc1 ; c1ccc(-c2cc(-c3ccccc3)cc(-c3ccc(N(c4ccccc4-c4ccccc4)c4cccc5oc6ccccc6c45)c4c3oc3ccccc34)c2)cc1 ; c1ccc(-c2cc(-c3ccccc3)cc(-c3ccc(N(c4ccc5c(c4)oc4ccccc45)c4ccccc4-c4ccccc4)c4c3oc3ccccc34)c2)cc1]

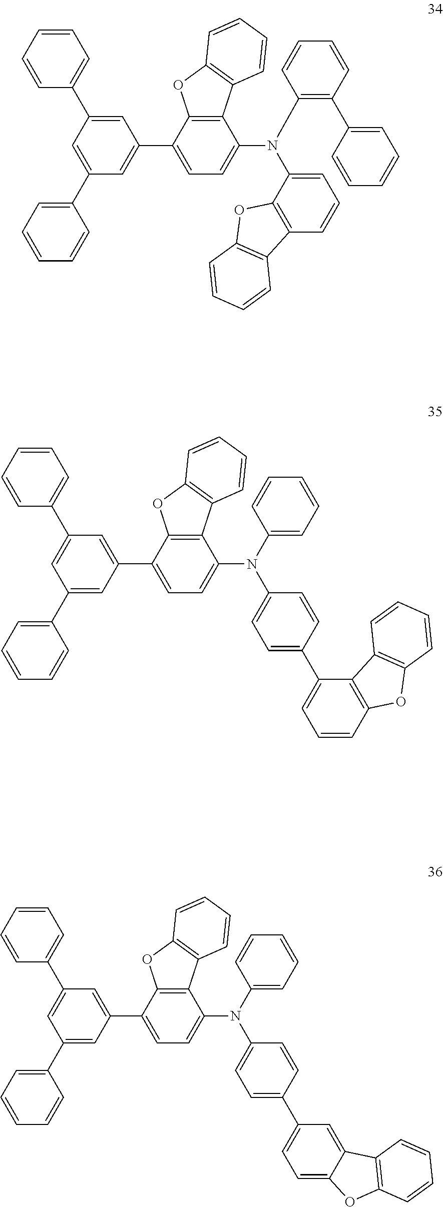 [SMILES: c1ccc(-c2cc(-c3ccccc3)cc(-c3ccc(N(c4ccccc4-c4ccccc4)c4cccc5c4oc4ccccc45)c4c3oc3ccccc34)c2)cc1 ; c1ccc(-c2cc(-c3ccccc3)cc(-c3ccc(N(c4ccccc4)c4ccc(-c5cccc6oc7ccccc7c56)cc4)c4c3oc3ccccc34)c2)cc1 ; c1ccc(-c2cc(-c3ccccc3)cc(-c3ccc(N(c4ccccc4)c4ccc(-c5ccc6oc7ccccc7c6c5)cc4)c4c3oc3ccccc34)c2)cc1]

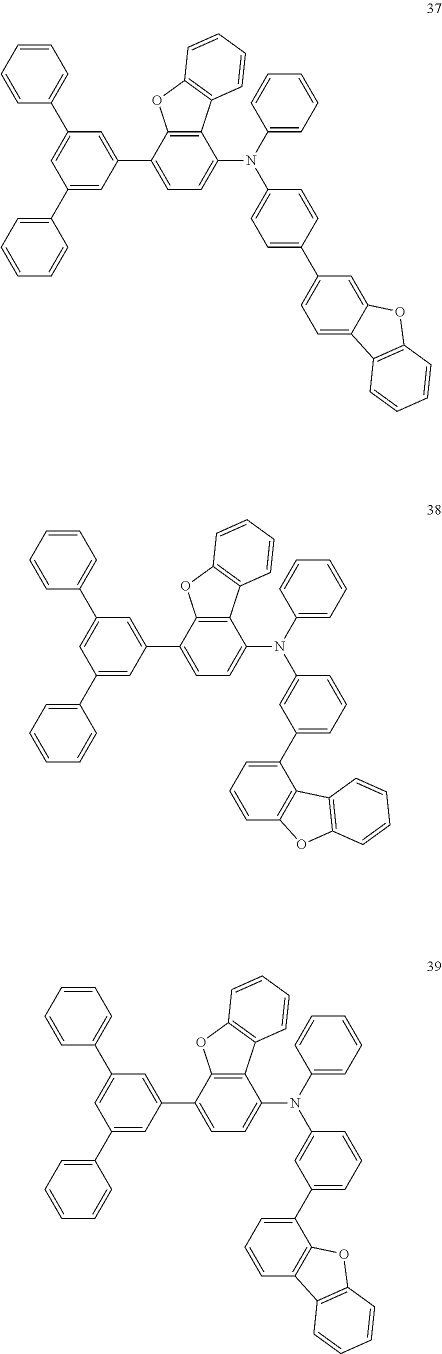 [SMILES: c1ccc(-c2cc(-c3ccccc3)cc(-c3ccc(N(c4ccccc4)c4ccc(-c5ccc6c(c5)oc5ccccc56)cc4)c4c3oc3ccccc34)c2)cc1 ; c1ccc(-c2cc(-c3ccccc3)cc(-c3ccc(N(c4ccccc4)c4cccc(-c5cccc6oc7ccccc7c56)c4)c4c3oc3ccccc34)c2)cc1 ; c1ccc(-c2cc(-c3ccccc3)cc(-c3ccc(N(c4ccccc4)c4cccc(-c5cccc6c5oc5ccccc56)c4)c4c3oc3ccccc34)c2)cc1]

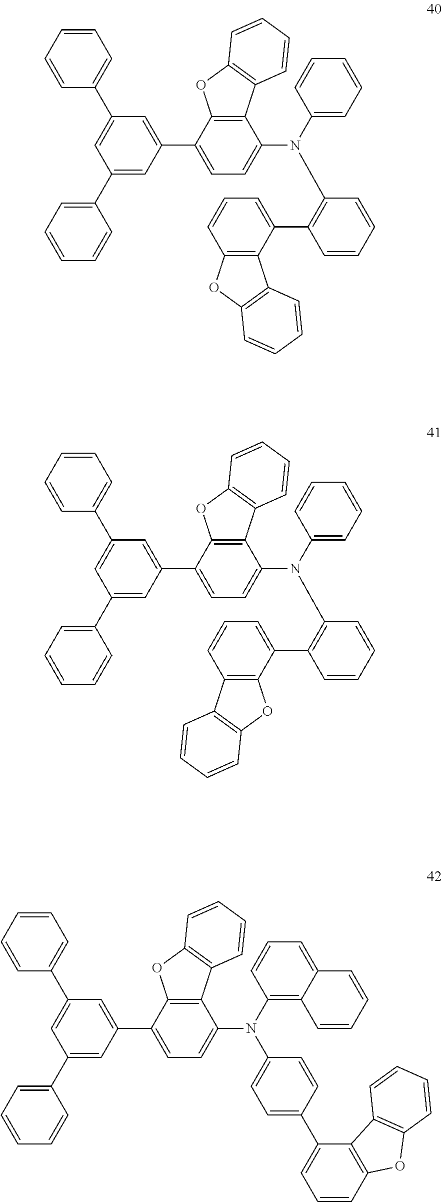 [SMILES: c1ccc(-c2cc(-c3ccccc3)cc(-c3ccc(N(c4ccccc4)c4ccccc4-c4cccc5oc6ccccc6c45)c4c3oc3ccccc34)c2)cc1 ; c1ccc(-c2cc(-c3ccccc3)cc(-c3ccc(N(c4ccccc4)c4ccccc4-c4cccc5c4oc4ccccc45)c4c3oc3ccccc34)c2)cc1 ; c1ccc(-c2cc(-c3ccccc3)cc(-c3ccc(N(c4ccc(-c5cccc6oc7ccccc7c56)cc4)c4cccc5ccccc45)c4c3oc3ccccc34)c2)cc1]

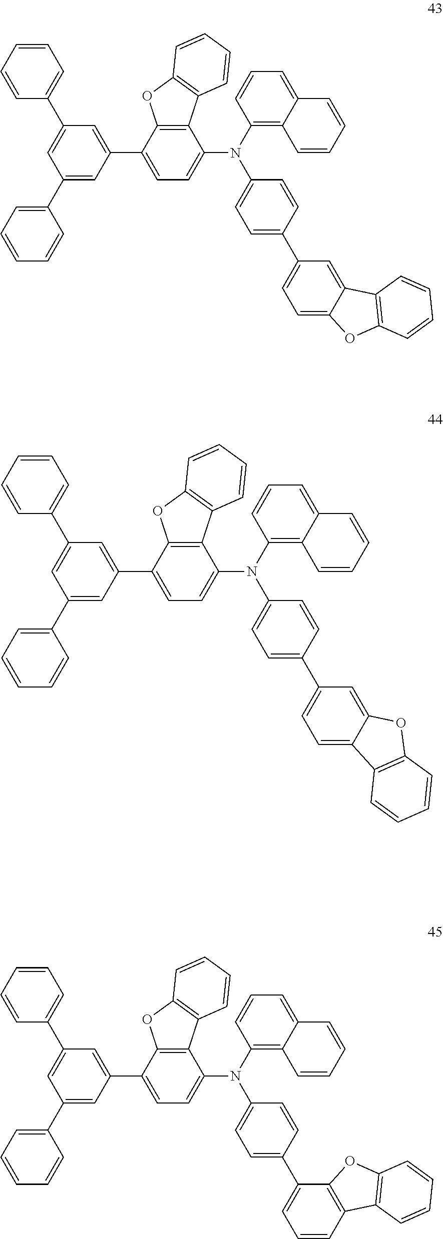 [SMILES: c1ccc(-c2cc(-c3ccccc3)cc(-c3ccc(N(c4ccc(-c5ccc6oc7ccccc7c6c5)cc4)c4cccc5ccccc45)c4c3oc3ccccc34)c2)cc1 ; c1ccc(-c2cc(-c3ccccc3)cc(-c3ccc(N(c4ccc(-c5ccc6c(c5)oc5ccccc56)cc4)c4cccc5ccccc45)c4c3oc3ccccc34)c2)cc1 ; c1ccc(-c2cc(-c3ccccc3)cc(-c3ccc(N(c4ccc(-c5cccc6c5oc5ccccc56)cc4)c4cccc5ccccc45)c4c3oc3ccccc34)c2)cc1]

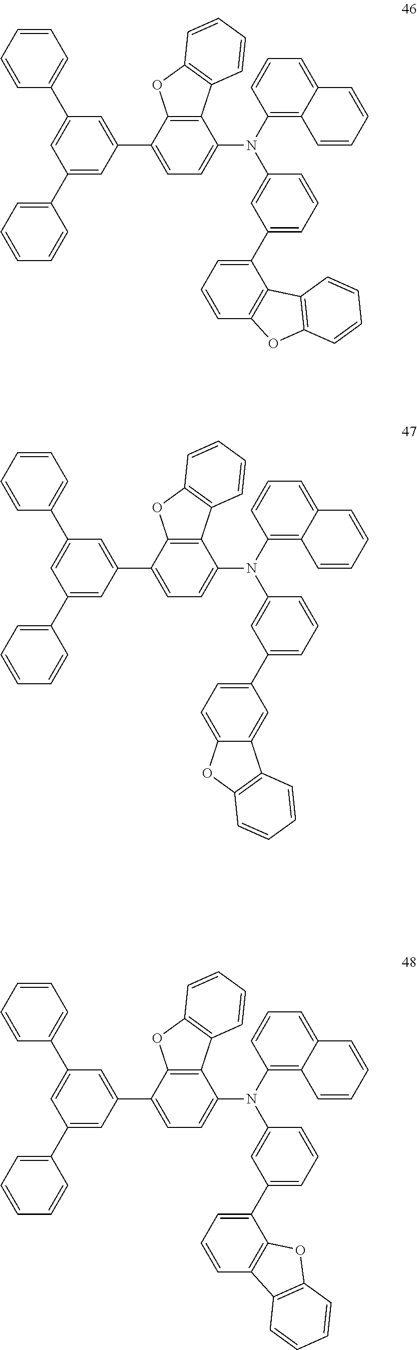 [SMILES: c1ccc(-c2cc(-c3ccccc3)cc(-c3ccc(N(c4cccc(-c5cccc6oc7ccccc7c56)c4)c4cccc5ccccc45)c4c3oc3ccccc34)c2)cc1 ; c1ccc(-c2cc(-c3ccccc3)cc(-c3ccc(N(c4cccc(-c5ccc6oc7ccccc7c6c5)c4)c4cccc5ccccc45)c4c3oc3ccccc34)c2)cc1 ; c1ccc(-c2cc(-c3ccccc3)cc(-c3ccc(N(c4cccc(-c5cccc6c5oc5ccccc56)c4)c4cccc5ccccc45)c4c3oc3ccccc34)c2)cc1]

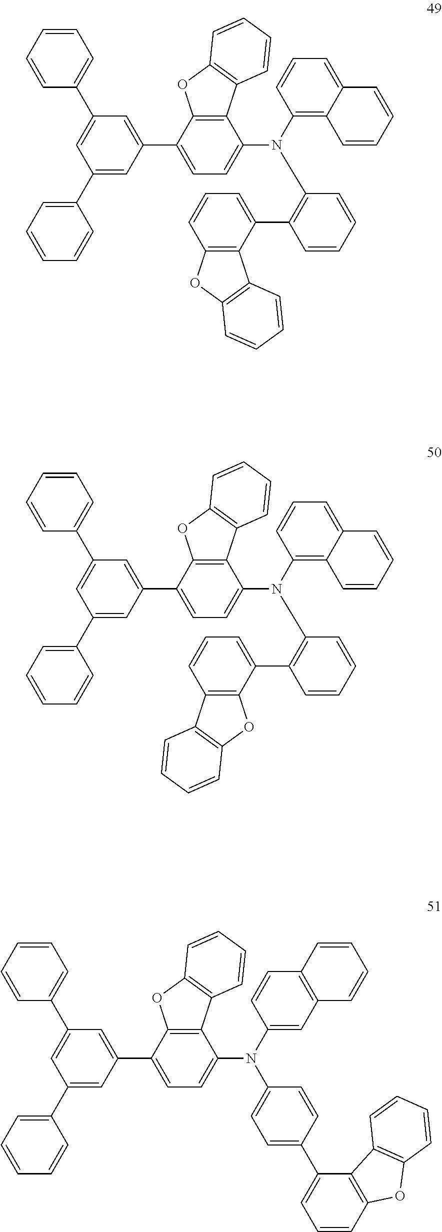 [SMILES: c1ccc(-c2cc(-c3ccccc3)cc(-c3ccc(N(c4ccccc4-c4cccc5oc6ccccc6c45)c4cccc5ccccc45)c4c3oc3ccccc34)c2)cc1 ; c1ccc(-c2cc(-c3ccccc3)cc(-c3ccc(N(c4ccccc4-c4cccc5c4oc4ccccc45)c4cccc5ccccc45)c4c3oc3ccccc34)c2)cc1 ; c1ccc(-c2cc(-c3ccccc3)cc(-c3ccc(N(c4ccc(-c5cccc6oc7ccccc7c56)cc4)c4ccc5ccccc5c4)c4c3oc3ccccc34)c2)cc1]

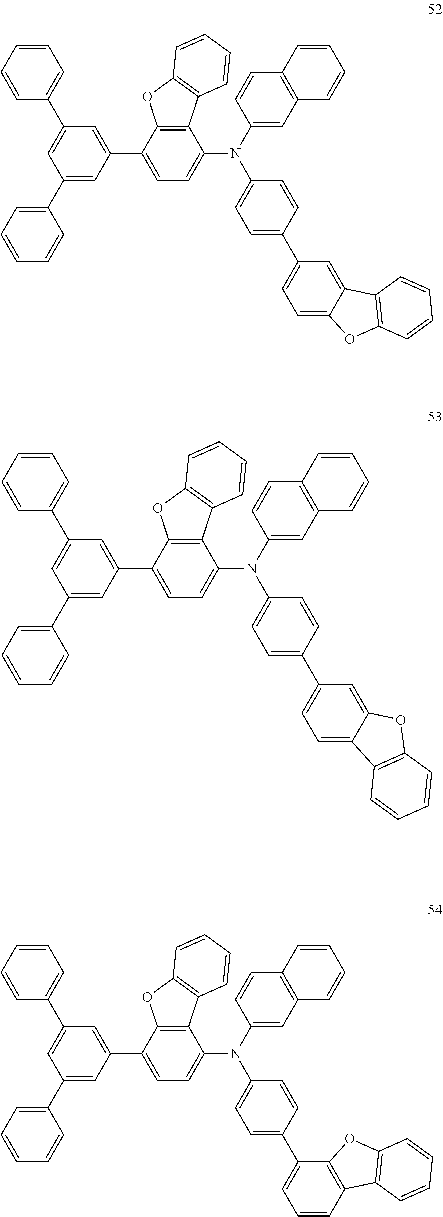 [SMILES: c1ccc(-c2cc(-c3ccccc3)cc(-c3ccc(N(c4ccc(-c5ccc6oc7ccccc7c6c5)cc4)c4ccc5ccccc5c4)c4c3oc3ccccc34)c2)cc1 ; c1ccc(-c2cc(-c3ccccc3)cc(-c3ccc(N(c4ccc(-c5ccc6c(c5)oc5ccccc56)cc4)c4ccc5ccccc5c4)c4c3oc3ccccc34)c2)cc1 ; c1ccc(-c2cc(-c3ccccc3)cc(-c3ccc(N(c4ccc(-c5cccc6c5oc5ccccc56)cc4)c4ccc5ccccc5c4)c4c3oc3ccccc34)c2)cc1]

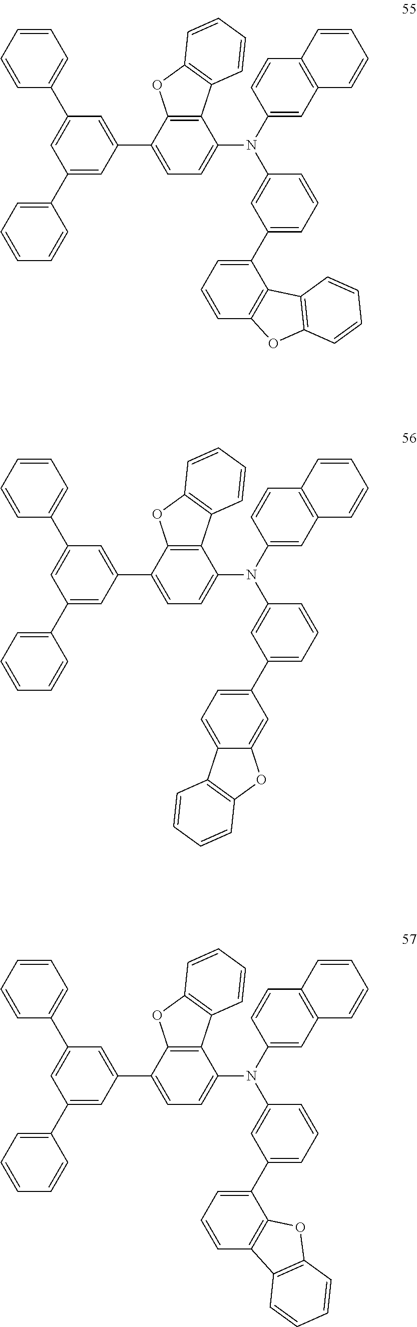 [SMILES: c1ccc(-c2cc(-c3ccccc3)cc(-c3ccc(N(c4cccc(-c5cccc6oc7ccccc7c56)c4)c4ccc5ccccc5c4)c4c3oc3ccccc34)c2)cc1 ; c1ccc(-c2cc(-c3ccccc3)cc(-c3ccc(N(c4cccc(-c5ccc6c(c5)oc5ccccc56)c4)c4ccc5ccccc5c4)c4c3oc3ccccc34)c2)cc1 ; c1ccc(-c2cc(-c3ccccc3)cc(-c3ccc(N(c4cccc(-c5cccc6c5oc5ccccc56)c4)c4ccc5ccccc5c4)c4c3oc3ccccc34)c2)cc1]

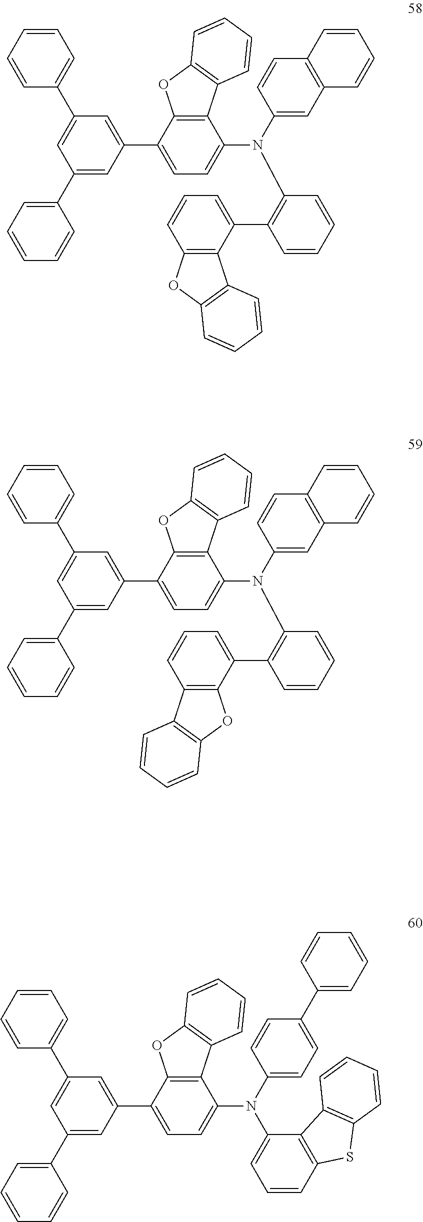 [SMILES: c1ccc(-c2cc(-c3ccccc3)cc(-c3ccc(N(c4ccc5ccccc5c4)c4ccccc4-c4cccc5oc6ccccc6c45)c4c3oc3ccccc34)c2)cc1 ; c1ccc(-c2cc(-c3ccccc3)cc(-c3ccc(N(c4ccc5ccccc5c4)c4ccccc4-c4cccc5c4oc4ccccc45)c4c3oc3ccccc34)c2)cc1 ; c1ccc(-c2ccc(N(c3cccc4sc5ccccc5c34)c3ccc(-c4cc(-c5ccccc5)cc(-c5ccccc5)c4)c4oc5ccccc5c34)cc2)cc1]

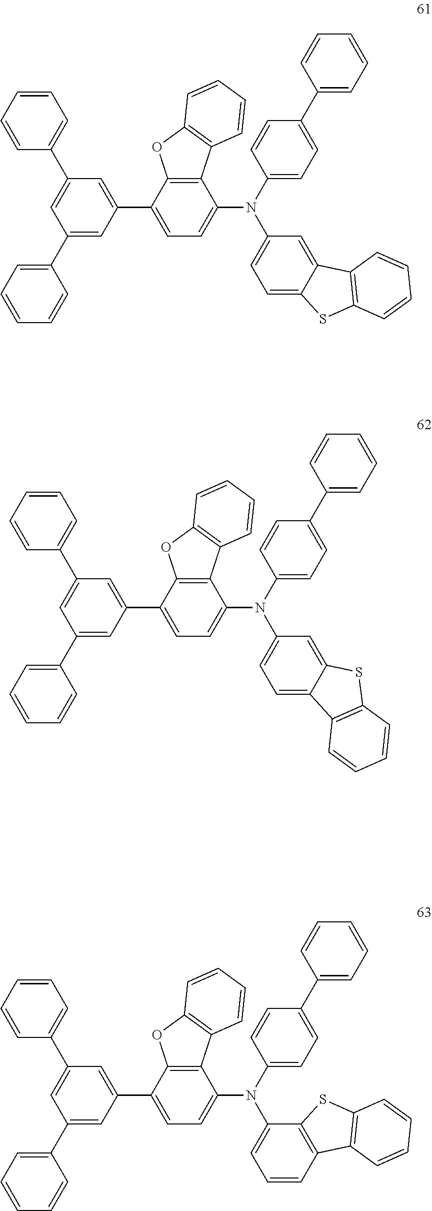 [SMILES: c1ccc(-c2ccc(N(c3ccc4sc5ccccc5c4c3)c3ccc(-c4cc(-c5ccccc5)cc(-c5ccccc5)c4)c4oc5ccccc5c34)cc2)cc1 ; c1ccc(-c2ccc(N(c3ccc4c(c3)sc3ccccc34)c3ccc(-c4cc(-c5ccccc5)cc(-c5ccccc5)c4)c4oc5ccccc5c34)cc2)cc1 ; c1ccc(-c2ccc(N(c3cccc4c3sc3ccccc34)c3ccc(-c4cc(-c5ccccc5)cc(-c5ccccc5)c4)c4oc5ccccc5c34)cc2)cc1]

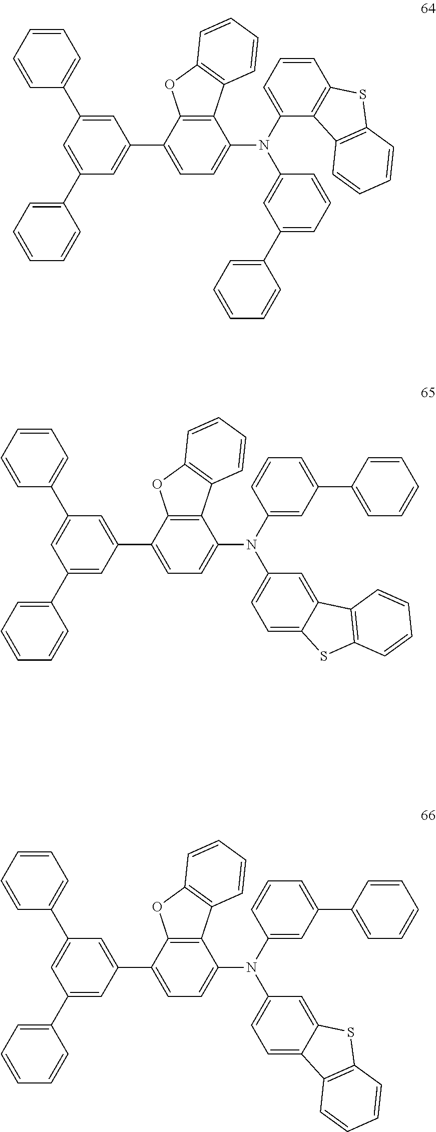 [SMILES: c1ccc(-c2cc(-c3ccccc3)cc(-c3ccc(N(c4cccc(-c5ccccc5)c4)c4cccc5sc6ccccc6c45)c4c3oc3ccccc34)c2)cc1 ; c1ccc(-c2cc(-c3ccccc3)cc(-c3ccc(N(c4cccc(-c5ccccc5)c4)c4ccc5sc6ccccc6c5c4)c4c3oc3ccccc34)c2)cc1 ; c1ccc(-c2cc(-c3ccccc3)cc(-c3ccc(N(c4cccc(-c5ccccc5)c4)c4ccc5c(c4)sc4ccccc45)c4c3oc3ccccc34)c2)cc1]

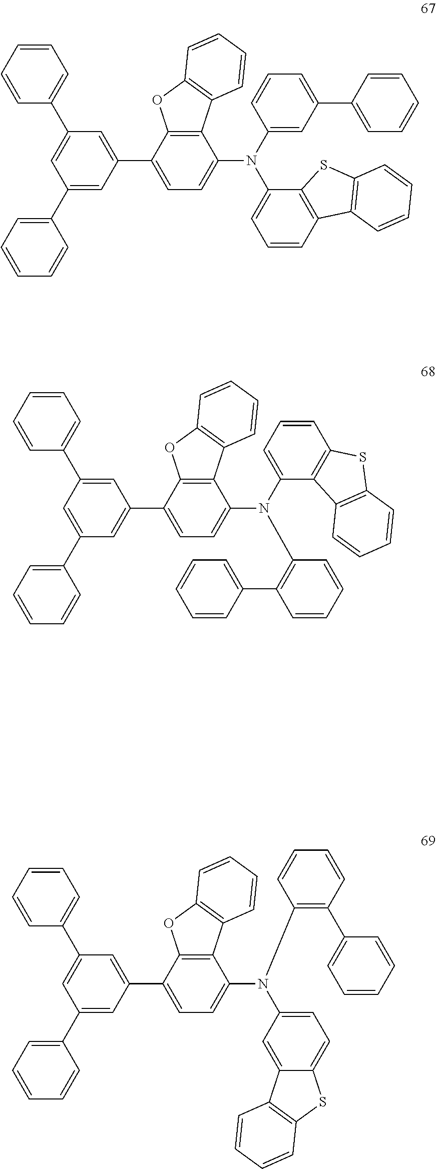 [SMILES: c1ccc(-c2cc(-c3ccccc3)cc(-c3ccc(N(c4cccc(-c5ccccc5)c4)c4cccc5c4sc4ccccc45)c4c3oc3ccccc34)c2)cc1 ; c1ccc(-c2cc(-c3ccccc3)cc(-c3ccc(N(c4ccccc4-c4ccccc4)c4cccc5sc6ccccc6c45)c4c3oc3ccccc34)c2)cc1 ; c1ccc(-c2cc(-c3ccccc3)cc(-c3ccc(N(c4ccc5sc6ccccc6c5c4)c4ccccc4-c4ccccc4)c4c3oc3ccccc34)c2)cc1]

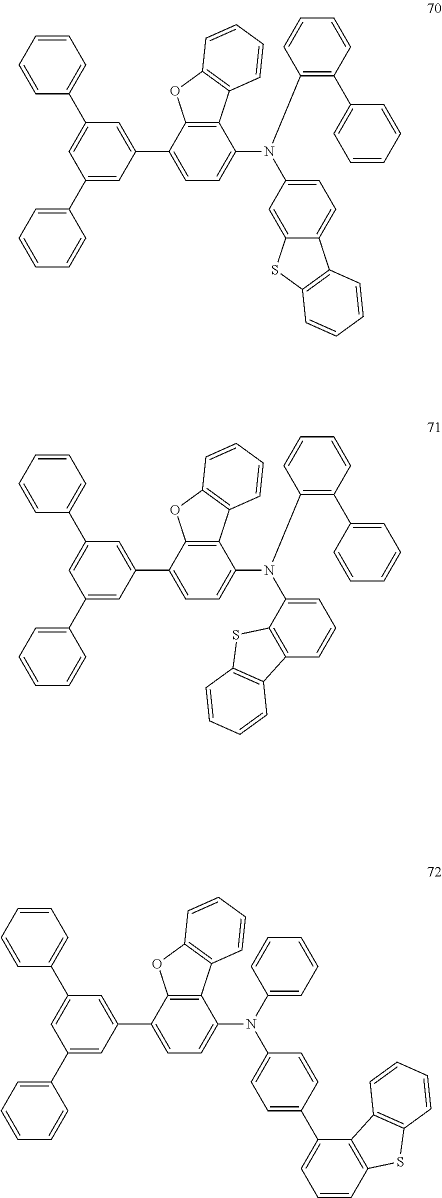 [SMILES: c1ccc(-c2cc(-c3ccccc3)cc(-c3ccc(N(c4ccc5c(c4)sc4ccccc45)c4ccccc4-c4ccccc4)c4c3oc3ccccc34)c2)cc1 ; c1ccc(-c2cc(-c3ccccc3)cc(-c3ccc(N(c4ccccc4-c4ccccc4)c4cccc5c4sc4ccccc45)c4c3oc3ccccc34)c2)cc1 ; c1ccc(-c2cc(-c3ccccc3)cc(-c3ccc(N(c4ccccc4)c4ccc(-c5cccc6sc7ccccc7c56)cc4)c4c3oc3ccccc34)c2)cc1]

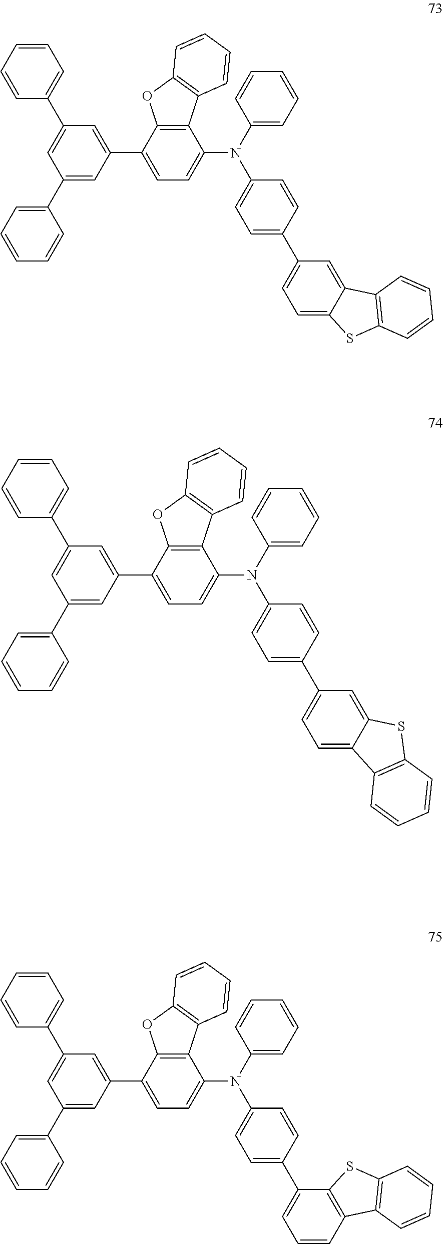 [SMILES: c1ccc(-c2cc(-c3ccccc3)cc(-c3ccc(N(c4ccccc4)c4ccc(-c5ccc6sc7ccccc7c6c5)cc4)c4c3oc3ccccc34)c2)cc1 ; c1ccc(-c2cc(-c3ccccc3)cc(-c3ccc(N(c4ccccc4)c4ccc(-c5ccc6c(c5)sc5ccccc56)cc4)c4c3oc3ccccc34)c2)cc1 ; c1ccc(-c2cc(-c3ccccc3)cc(-c3ccc(N(c4ccccc4)c4ccc(-c5cccc6c5sc5ccccc56)cc4)c4c3oc3ccccc34)c2)cc1]

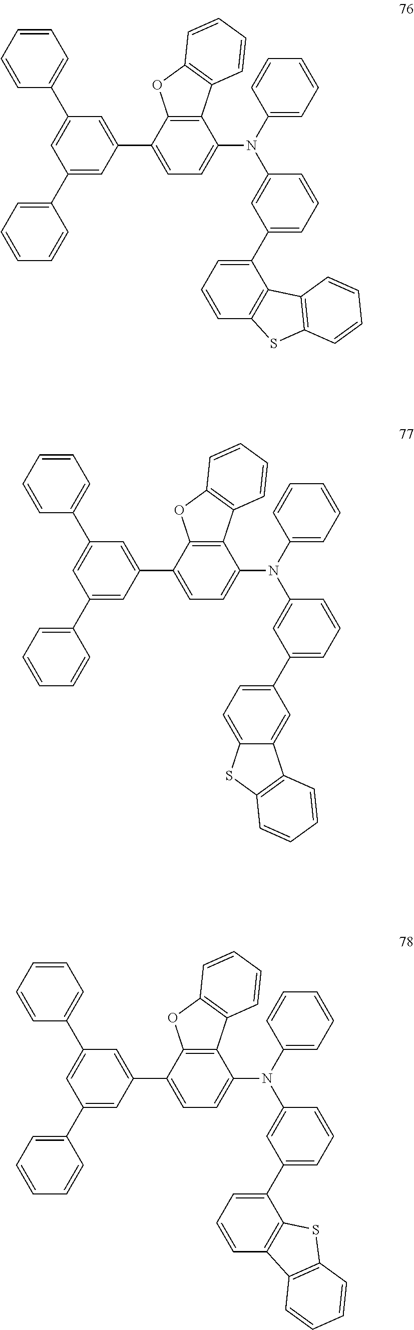 [SMILES: c1ccc(-c2cc(-c3ccccc3)cc(-c3ccc(N(c4ccccc4)c4cccc(-c5cccc6sc7ccccc7c56)c4)c4c3oc3ccccc34)c2)cc1 ; c1ccc(-c2cc(-c3ccccc3)cc(-c3ccc(N(c4ccccc4)c4cccc(-c5ccc6sc7ccccc7c6c5)c4)c4c3oc3ccccc34)c2)cc1 ; c1ccc(-c2cc(-c3ccccc3)cc(-c3ccc(N(c4ccccc4)c4cccc(-c5cccc6c5sc5ccccc56)c4)c4c3oc3ccccc34)c2)cc1]

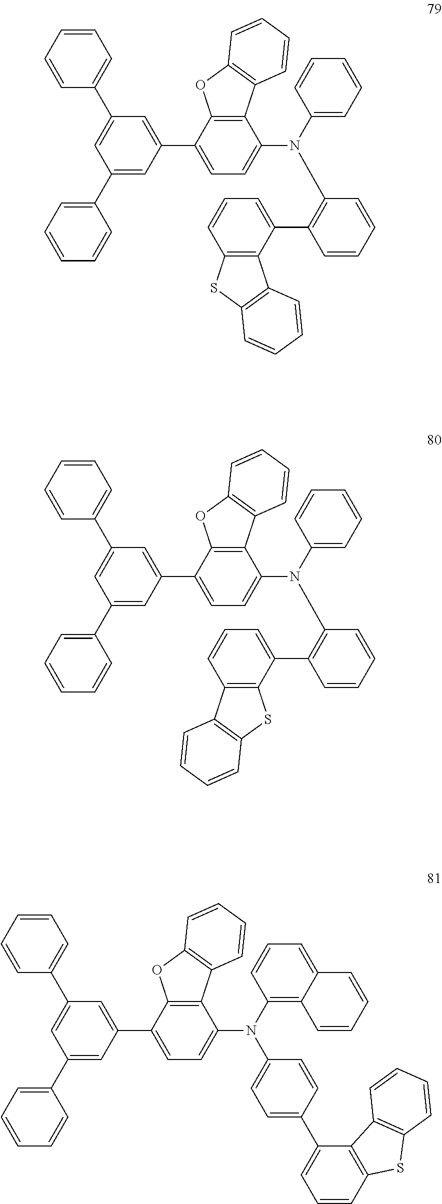 [SMILES: c1ccc(-c2cc(-c3ccccc3)cc(-c3ccc(N(c4ccccc4)c4ccccc4-c4cccc5sc6ccccc6c45)c4c3oc3ccccc34)c2)cc1 ; c1ccc(-c2cc(-c3ccccc3)cc(-c3ccc(N(c4ccccc4)c4ccccc4-c4cccc5c4sc4ccccc45)c4c3oc3ccccc34)c2)cc1 ; c1ccc(-c2cc(-c3ccccc3)cc(-c3ccc(N(c4ccc(-c5cccc6sc7ccccc7c56)cc4)c4cccc5ccccc45)c4c3oc3ccccc34)c2)cc1]

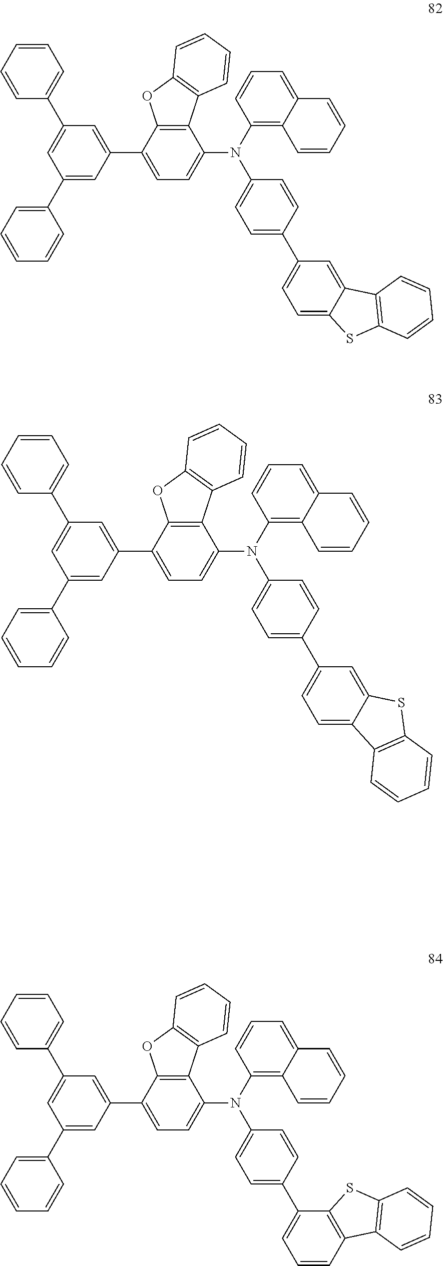 [SMILES: c1ccc(-c2cc(-c3ccccc3)cc(-c3ccc(N(c4ccc(-c5ccc6sc7ccccc7c6c5)cc4)c4cccc5ccccc45)c4c3oc3ccccc34)c2)cc1 ; c1ccc(-c2cc(-c3ccccc3)cc(-c3ccc(N(c4ccc(-c5ccc6c(c5)sc5ccccc56)cc4)c4cccc5ccccc45)c4c3oc3ccccc34)c2)cc1 ; c1ccc(-c2cc(-c3ccccc3)cc(-c3ccc(N(c4ccc(-c5cccc6c5sc5ccccc56)cc4)c4cccc5ccccc45)c4c3oc3ccccc34)c2)cc1]

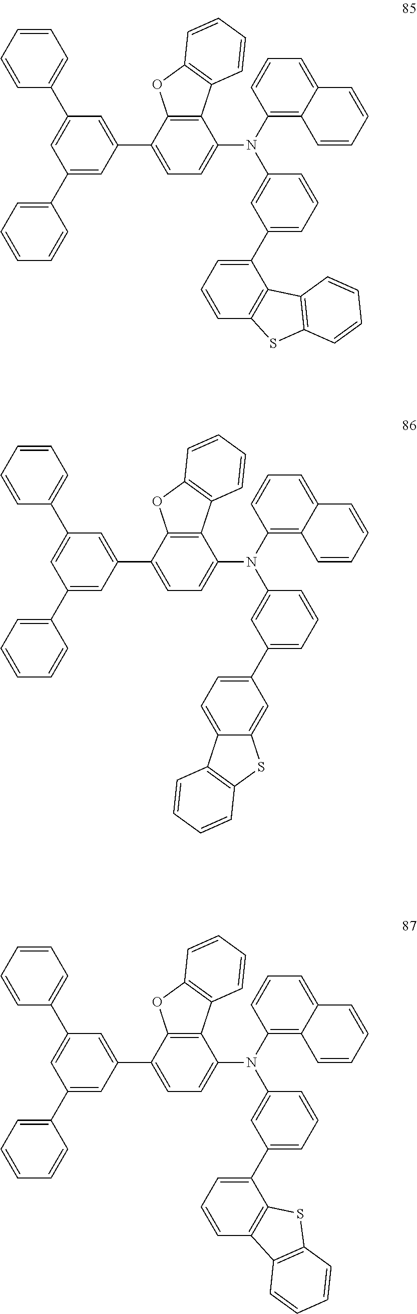 [SMILES: c1ccc(-c2cc(-c3ccccc3)cc(-c3ccc(N(c4cccc(-c5cccc6sc7ccccc7c56)c4)c4cccc5ccccc45)c4c3oc3ccccc34)c2)cc1 ; c1ccc(-c2cc(-c3ccccc3)cc(-c3ccc(N(c4cccc(-c5ccc6c(c5)sc5ccccc56)c4)c4cccc5ccccc45)c4c3oc3ccccc34)c2)cc1 ; c1ccc(-c2cc(-c3ccccc3)cc(-c3ccc(N(c4cccc(-c5cccc6c5sc5ccccc56)c4)c4cccc5ccccc45)c4c3oc3ccccc34)c2)cc1]

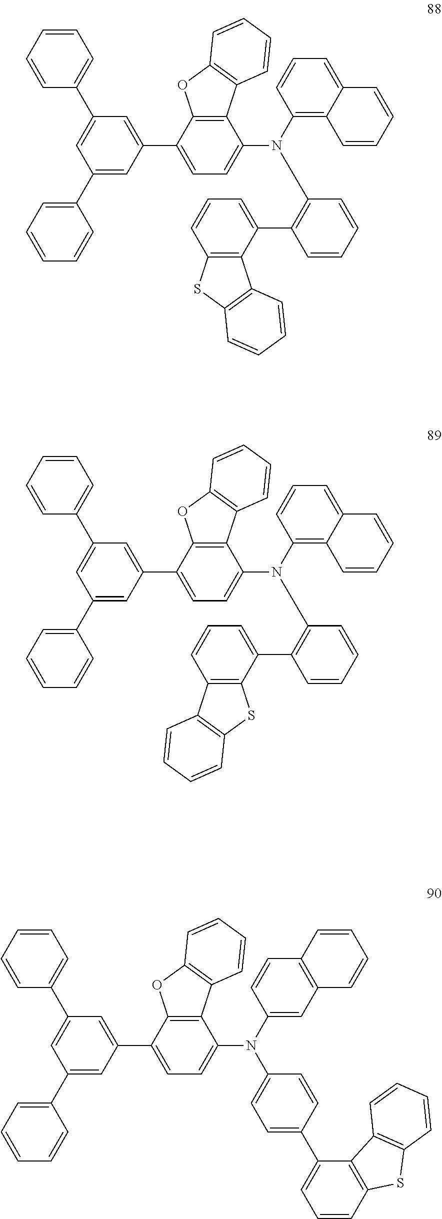 [SMILES: c1ccc(-c2cc(-c3ccccc3)cc(-c3ccc(N(c4ccccc4-c4cccc5sc6ccccc6c45)c4cccc5ccccc45)c4c3oc3ccccc34)c2)cc1 ; c1ccc(-c2cc(-c3ccccc3)cc(-c3ccc(N(c4ccccc4-c4cccc5c4sc4ccccc45)c4cccc5ccccc45)c4c3oc3ccccc34)c2)cc1 ; c1ccc(-c2cc(-c3ccccc3)cc(-c3ccc(N(c4ccc(-c5cccc6sc7ccccc7c56)cc4)c4ccc5ccccc5c4)c4c3oc3ccccc34)c2)cc1]

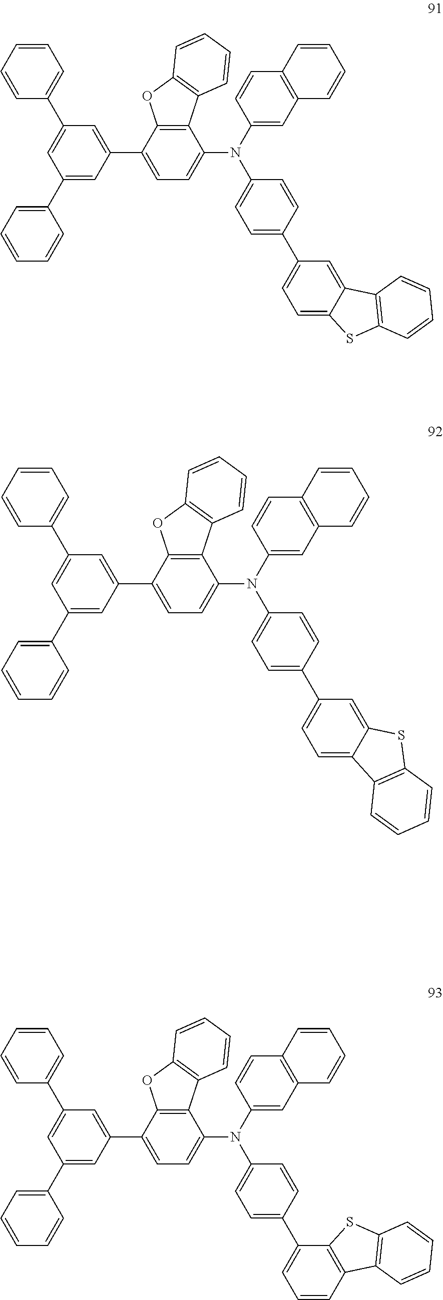 [SMILES: c1ccc(-c2cc(-c3ccccc3)cc(-c3ccc(N(c4ccc(-c5ccc6sc7ccccc7c6c5)cc4)c4ccc5ccccc5c4)c4c3oc3ccccc34)c2)cc1 ; c1ccc(-c2cc(-c3ccccc3)cc(-c3ccc(N(c4ccc(-c5ccc6c(c5)sc5ccccc56)cc4)c4ccc5ccccc5c4)c4c3oc3ccccc34)c2)cc1 ; c1ccc(-c2cc(-c3ccccc3)cc(-c3ccc(N(c4ccc(-c5cccc6c5sc5ccccc56)cc4)c4ccc5ccccc5c4)c4c3oc3ccccc34)c2)cc1]

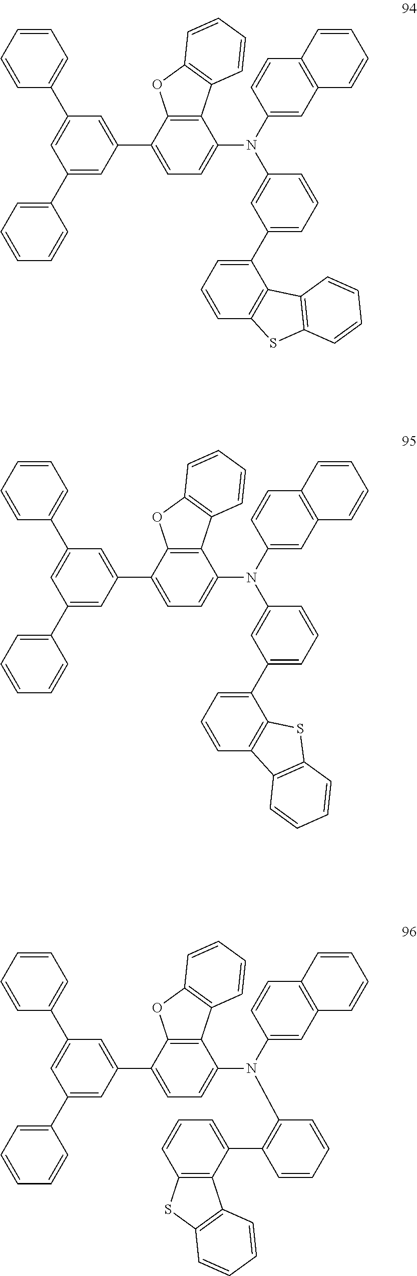 [SMILES: c1ccc(-c2cc(-c3ccccc3)cc(-c3ccc(N(c4cccc(-c5cccc6sc7ccccc7c56)c4)c4ccc5ccccc5c4)c4c3oc3ccccc34)c2)cc1 ; c1ccc(-c2cc(-c3ccccc3)cc(-c3ccc(N(c4cccc(-c5cccc6c5sc5ccccc56)c4)c4ccc5ccccc5c4)c4c3oc3ccccc34)c2)cc1 ; c1ccc(-c2cc(-c3ccccc3)cc(-c3ccc(N(c4ccc5ccccc5c4)c4ccccc4-c4cccc5sc6ccccc6c45)c4c3oc3ccccc34)c2)cc1]

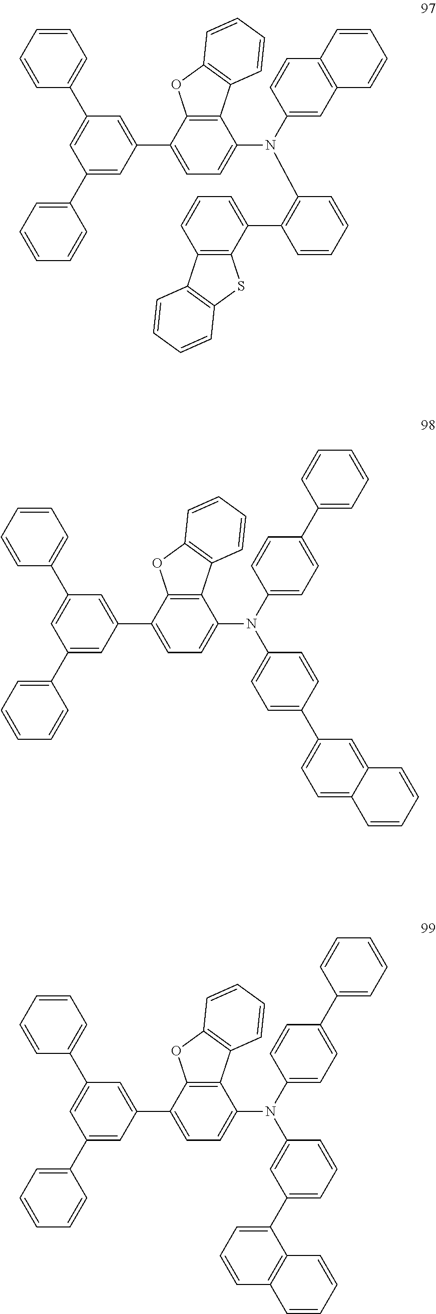 [SMILES: c1ccc(-c2cc(-c3ccccc3)cc(-c3ccc(N(c4ccc5ccccc5c4)c4ccccc4-c4cccc5c4sc4ccccc45)c4c3oc3ccccc34)c2)cc1 ; c1ccc(-c2ccc(N(c3ccc(-c4ccc5ccccc5c4)cc3)c3ccc(-c4cc(-c5ccccc5)cc(-c5ccccc5)c4)c4oc5ccccc5c34)cc2)cc1 ; c1ccc(-c2ccc(N(c3cccc(-c4cccc5ccccc45)c3)c3ccc(-c4cc(-c5ccccc5)cc(-c5ccccc5)c4)c4oc5ccccc5c34)cc2)cc1]

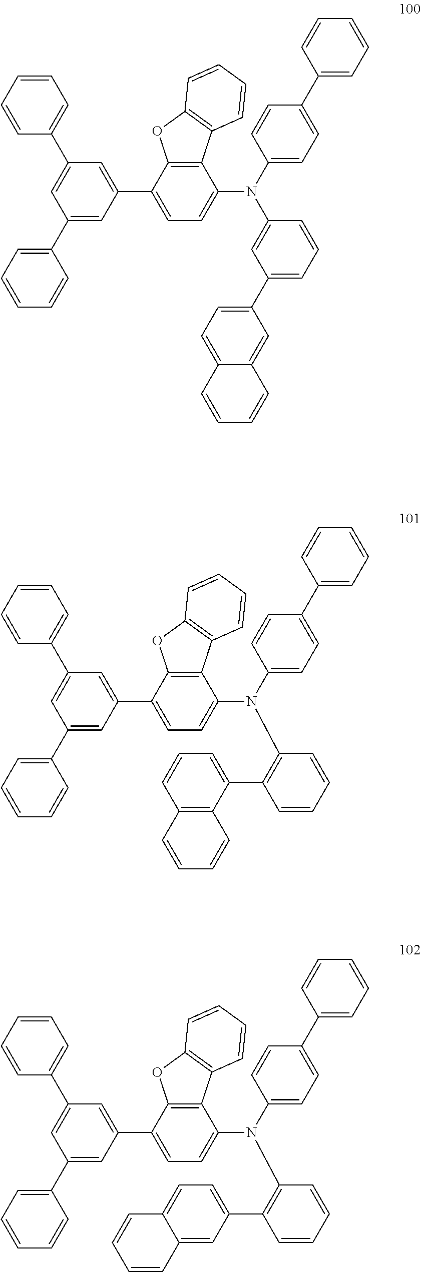 [SMILES: c1ccc(-c2ccc(N(c3cccc(-c4ccc5ccccc5c4)c3)c3ccc(-c4cc(-c5ccccc5)cc(-c5ccccc5)c4)c4oc5ccccc5c34)cc2)cc1 ; c1ccc(-c2ccc(N(c3ccccc3-c3cccc4ccccc34)c3ccc(-c4cc(-c5ccccc5)cc(-c5ccccc5)c4)c4oc5ccccc5c34)cc2)cc1 ; c1ccc(-c2ccc(N(c3ccccc3-c3ccc4ccccc4c3)c3ccc(-c4cc(-c5ccccc5)cc(-c5ccccc5)c4)c4oc5ccccc5c34)cc2)cc1]

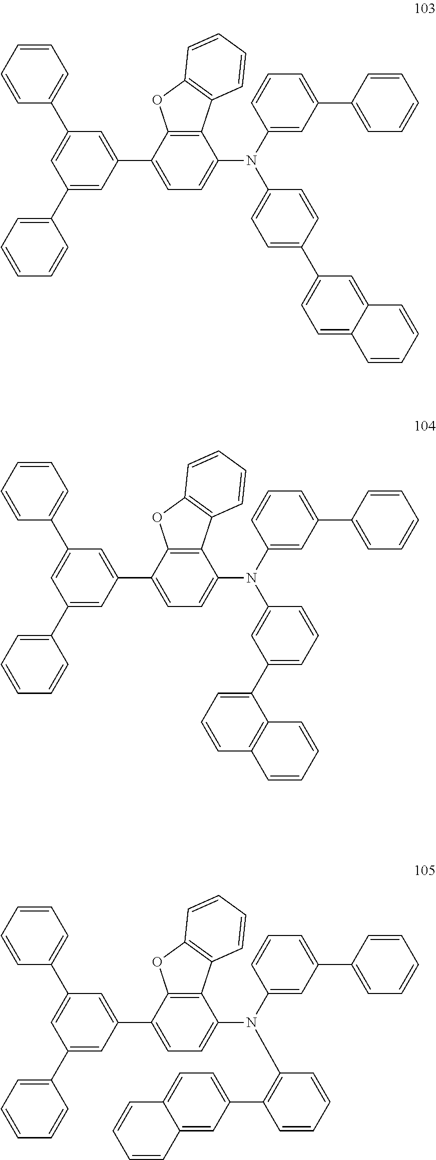 [SMILES: c1ccc(-c2cc(-c3ccccc3)cc(-c3ccc(N(c4ccc(-c5ccc6ccccc6c5)cc4)c4cccc(-c5ccccc5)c4)c4c3oc3ccccc34)c2)cc1 ; c1ccc(-c2cc(-c3ccccc3)cc(-c3ccc(N(c4cccc(-c5ccccc5)c4)c4cccc(-c5cccc6ccccc56)c4)c4c3oc3ccccc34)c2)cc1 ; c1ccc(-c2cc(-c3ccccc3)cc(-c3ccc(N(c4cccc(-c5ccccc5)c4)c4ccccc4-c4ccc5ccccc5c4)c4c3oc3ccccc34)c2)cc1]

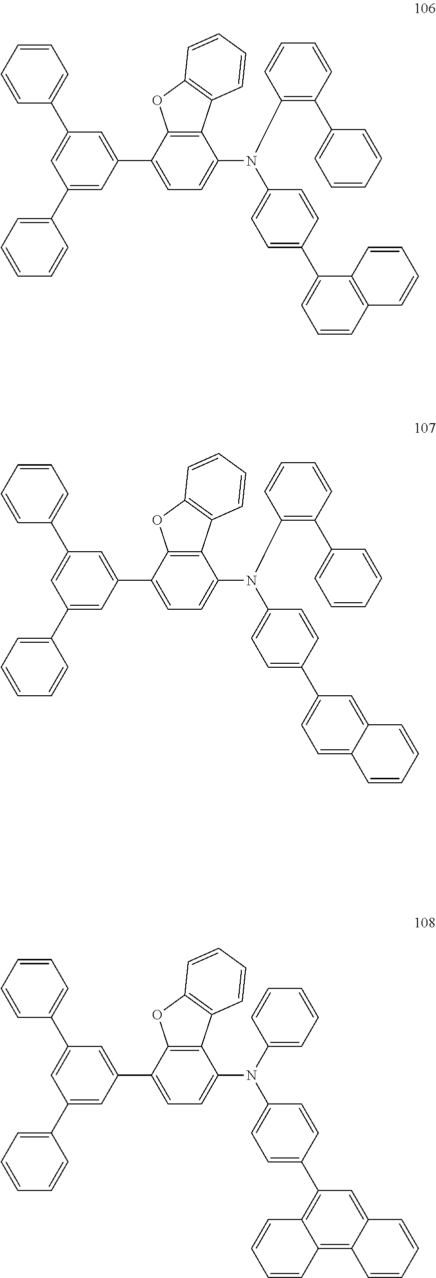 [SMILES: c1ccc(-c2cc(-c3ccccc3)cc(-c3ccc(N(c4ccc(-c5cccc6ccccc56)cc4)c4ccccc4-c4ccccc4)c4c3oc3ccccc34)c2)cc1 ; c1ccc(-c2cc(-c3ccccc3)cc(-c3ccc(N(c4ccc(-c5ccc6ccccc6c5)cc4)c4ccccc4-c4ccccc4)c4c3oc3ccccc34)c2)cc1 ; c1ccc(-c2cc(-c3ccccc3)cc(-c3ccc(N(c4ccccc4)c4ccc(-c5cc6ccccc6c6ccccc56)cc4)c4c3oc3ccccc34)c2)cc1]

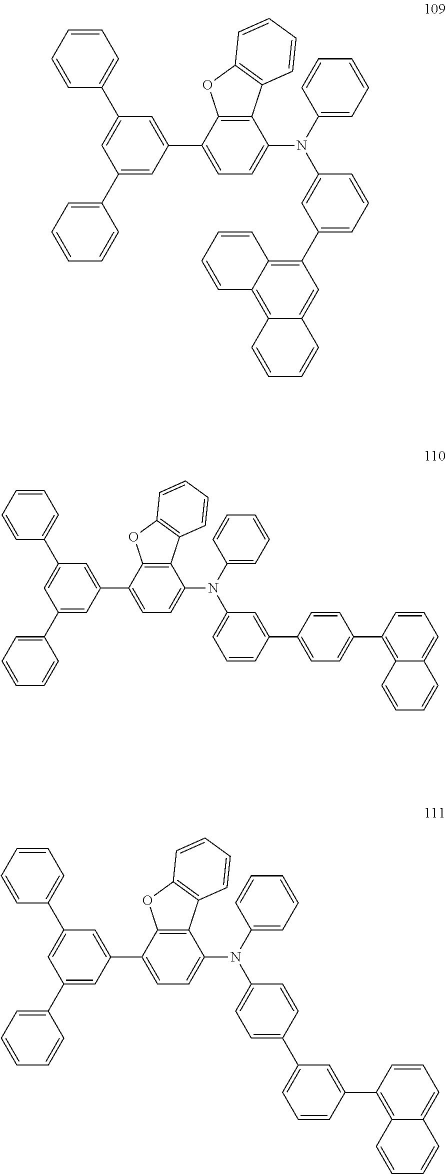 [SMILES: c1ccc(-c2cc(-c3ccccc3)cc(-c3ccc(N(c4ccccc4)c4cccc(-c5cc6ccccc6c6ccccc56)c4)c4c3oc3ccccc34)c2)cc1 ; c1ccc(-c2cc(-c3ccccc3)cc(-c3ccc(N(c4ccccc4)c4cccc(-c5ccc(-c6cccc7ccccc67)cc5)c4)c4c3oc3ccccc34)c2)cc1 ; c1ccc(-c2cc(-c3ccccc3)cc(-c3ccc(N(c4ccccc4)c4ccc(-c5cccc(-c6cccc7ccccc67)c5)cc4)c4c3oc3ccccc34)c2)cc1]

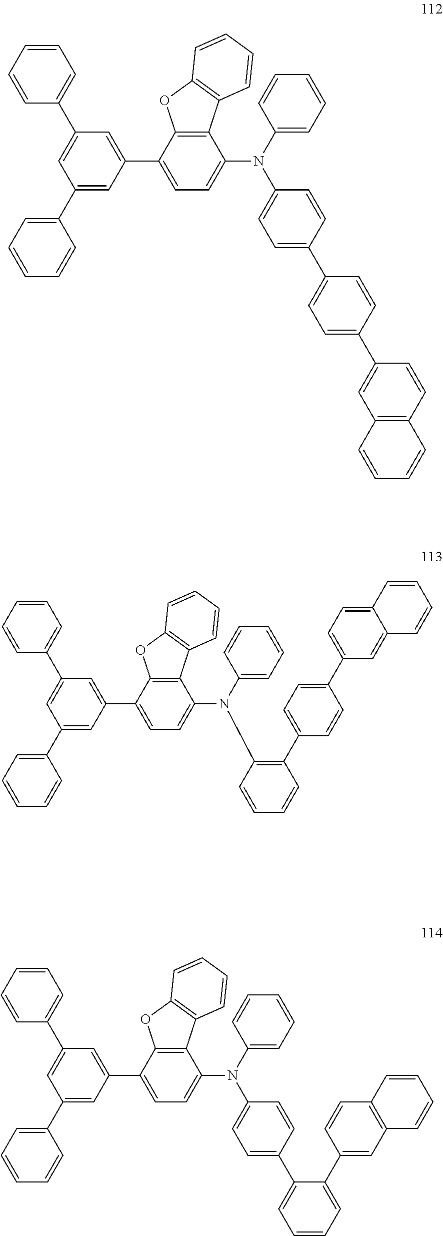 [SMILES: c1ccc(-c2cc(-c3ccccc3)cc(-c3ccc(N(c4ccccc4)c4ccc(-c5ccc(-c6ccc7ccccc7c6)cc5)cc4)c4c3oc3ccccc34)c2)cc1 ; c1ccc(-c2cc(-c3ccccc3)cc(-c3ccc(N(c4ccccc4)c4ccccc4-c4ccc(-c5ccc6ccccc6c5)cc4)c4c3oc3ccccc34)c2)cc1 ; c1ccc(-c2cc(-c3ccccc3)cc(-c3ccc(N(c4ccccc4)c4ccc(-c5ccccc5-c5ccc6ccccc6c5)cc4)c4c3oc3ccccc34)c2)cc1]

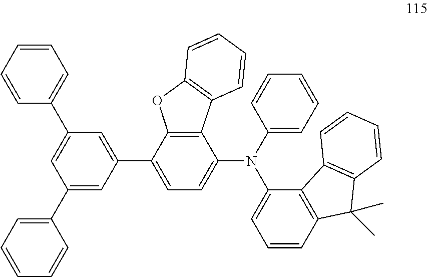 [SMILES: CC1(C)c2ccccc2-c2c(N(c3ccccc3)c3ccc(-c4cc(-c5ccccc5)cc(-c5ccccc5)c4)c4oc5ccccc5c34)cccc21]

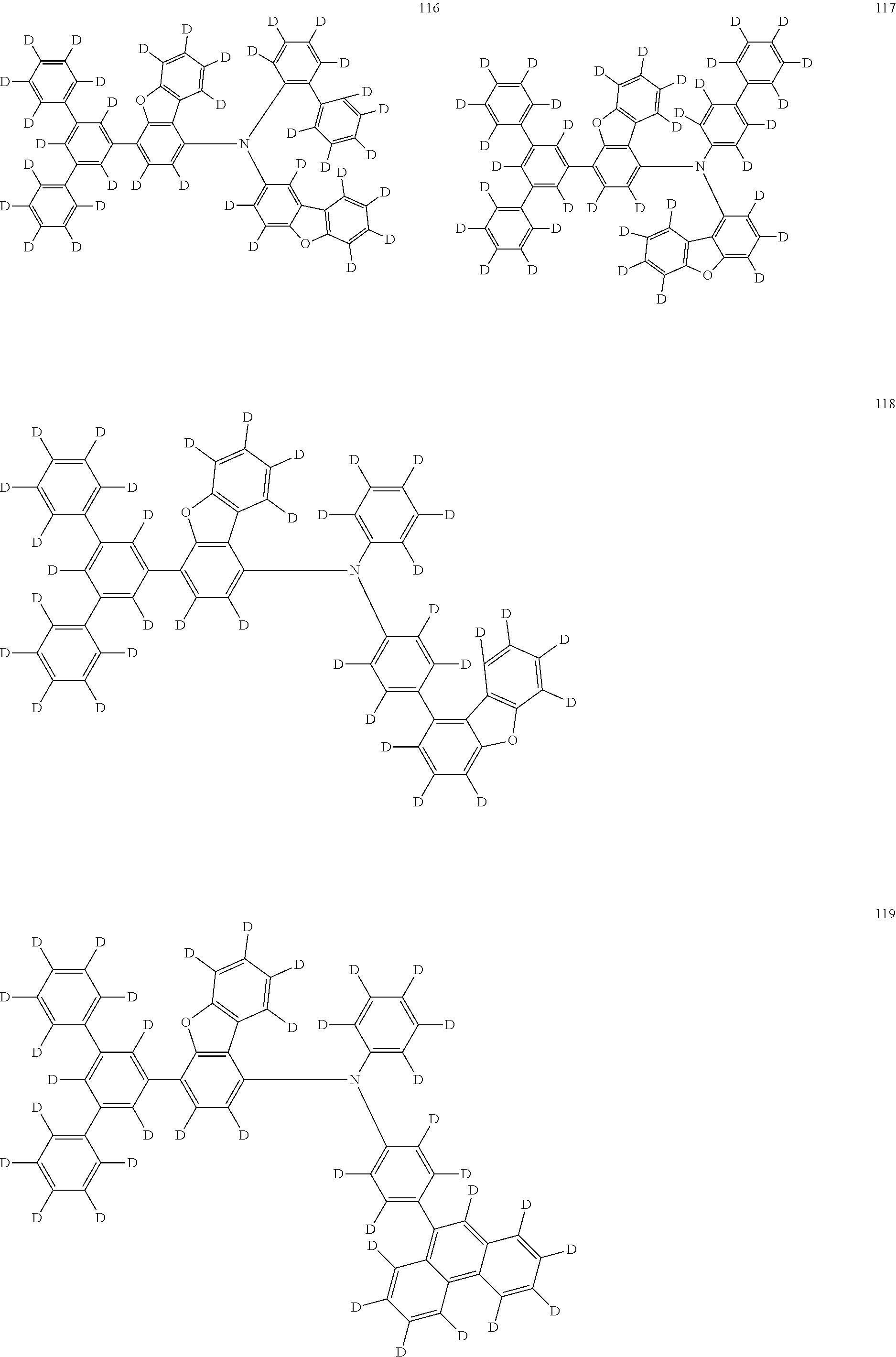

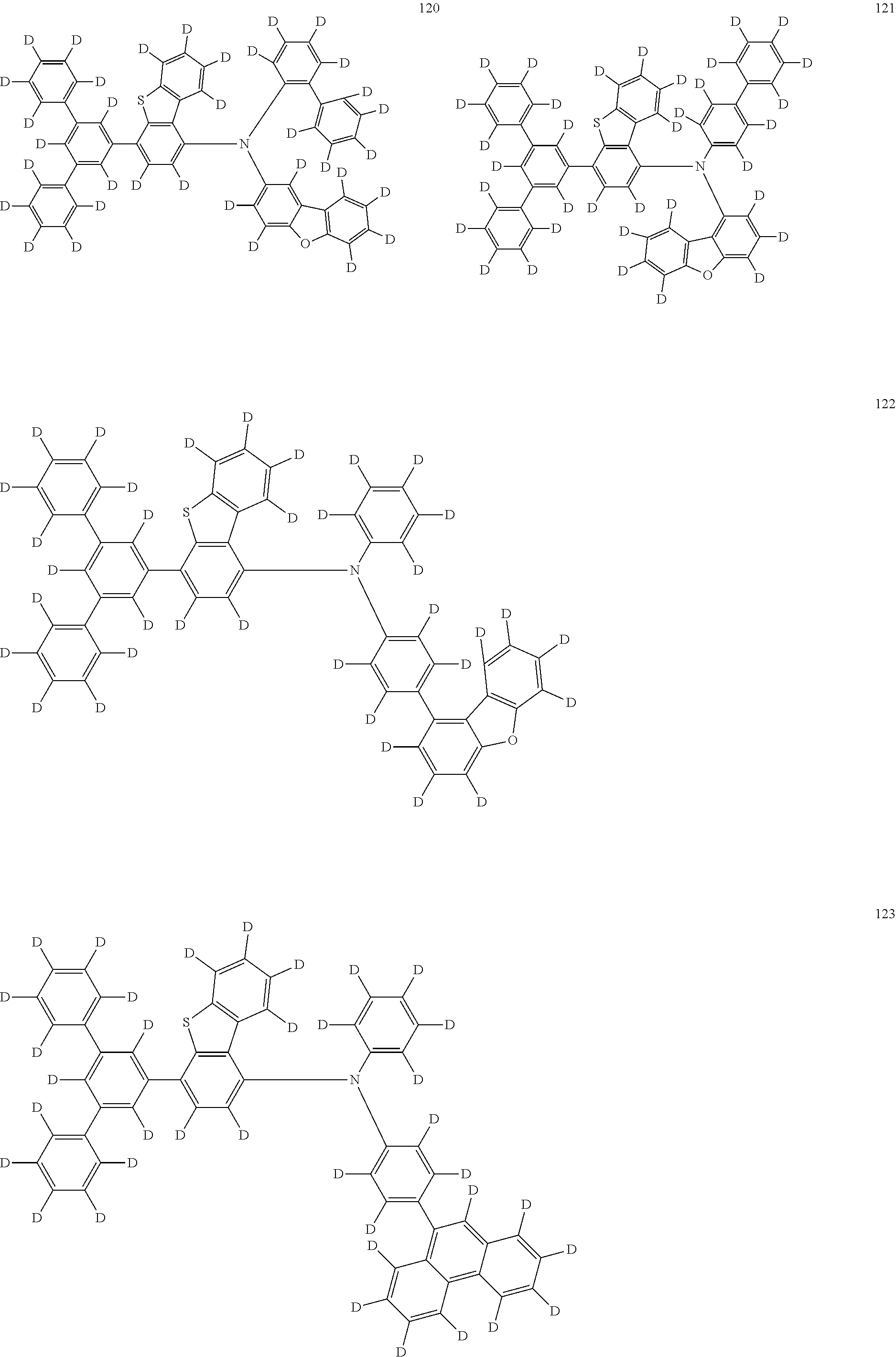

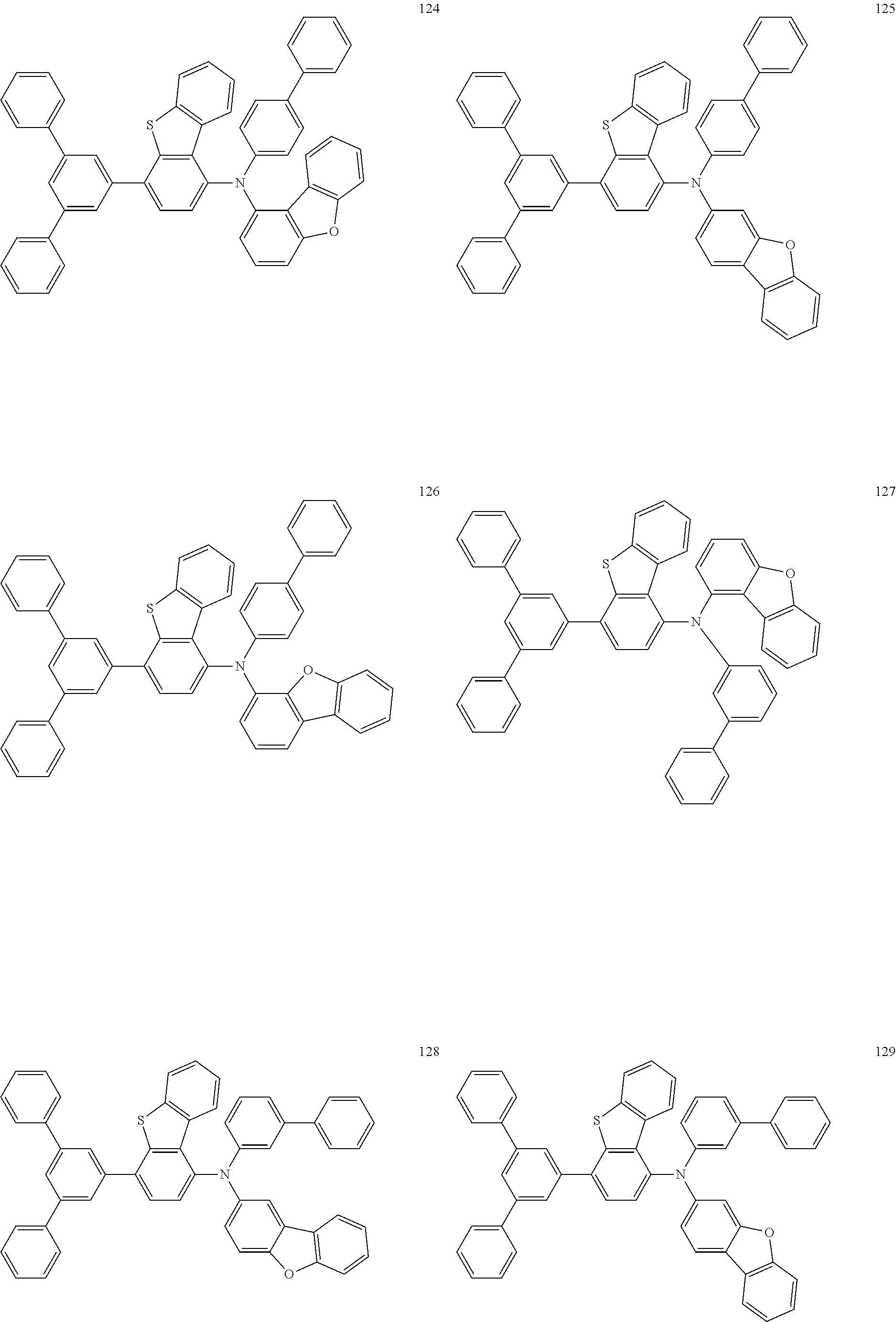

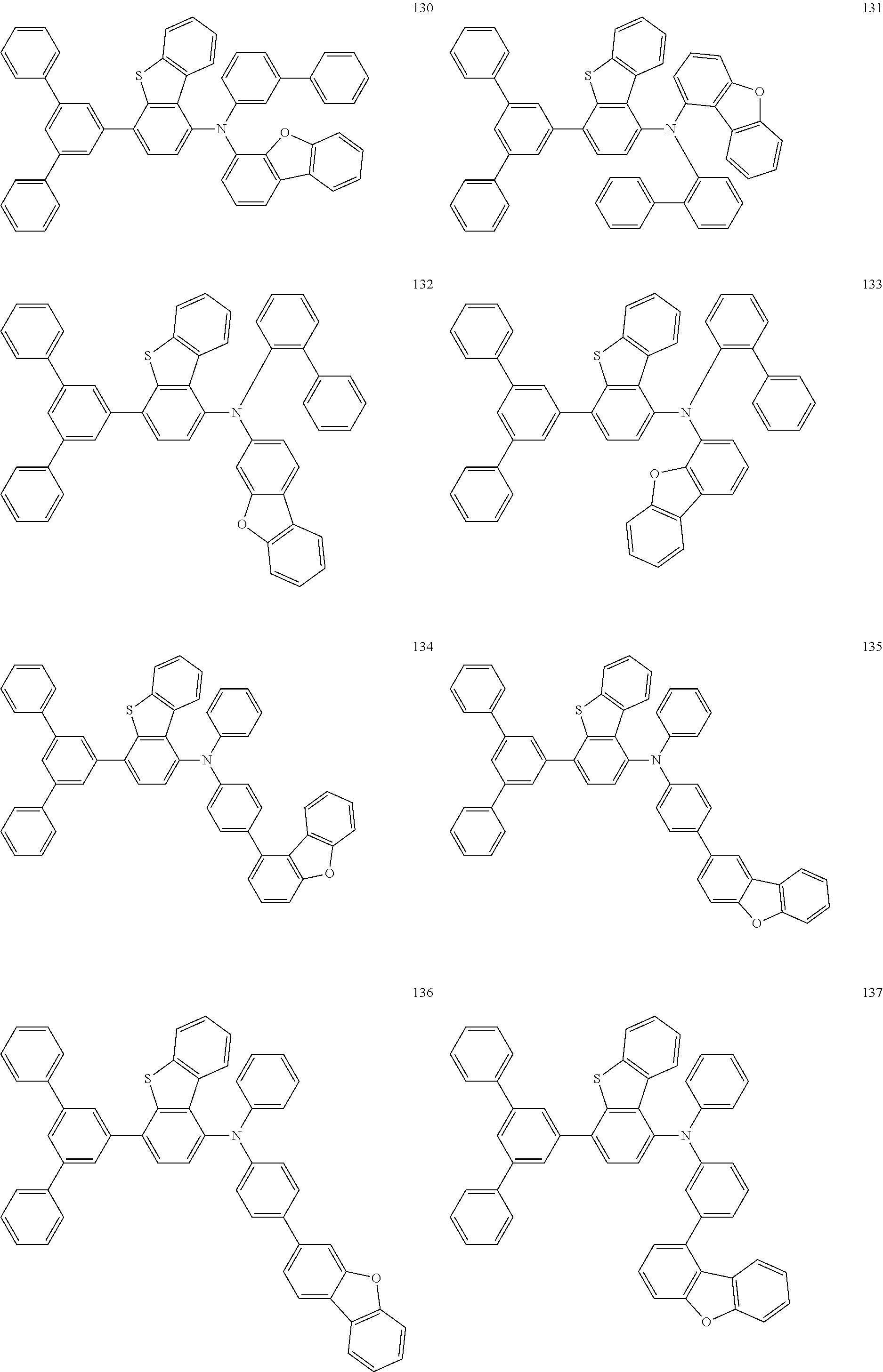

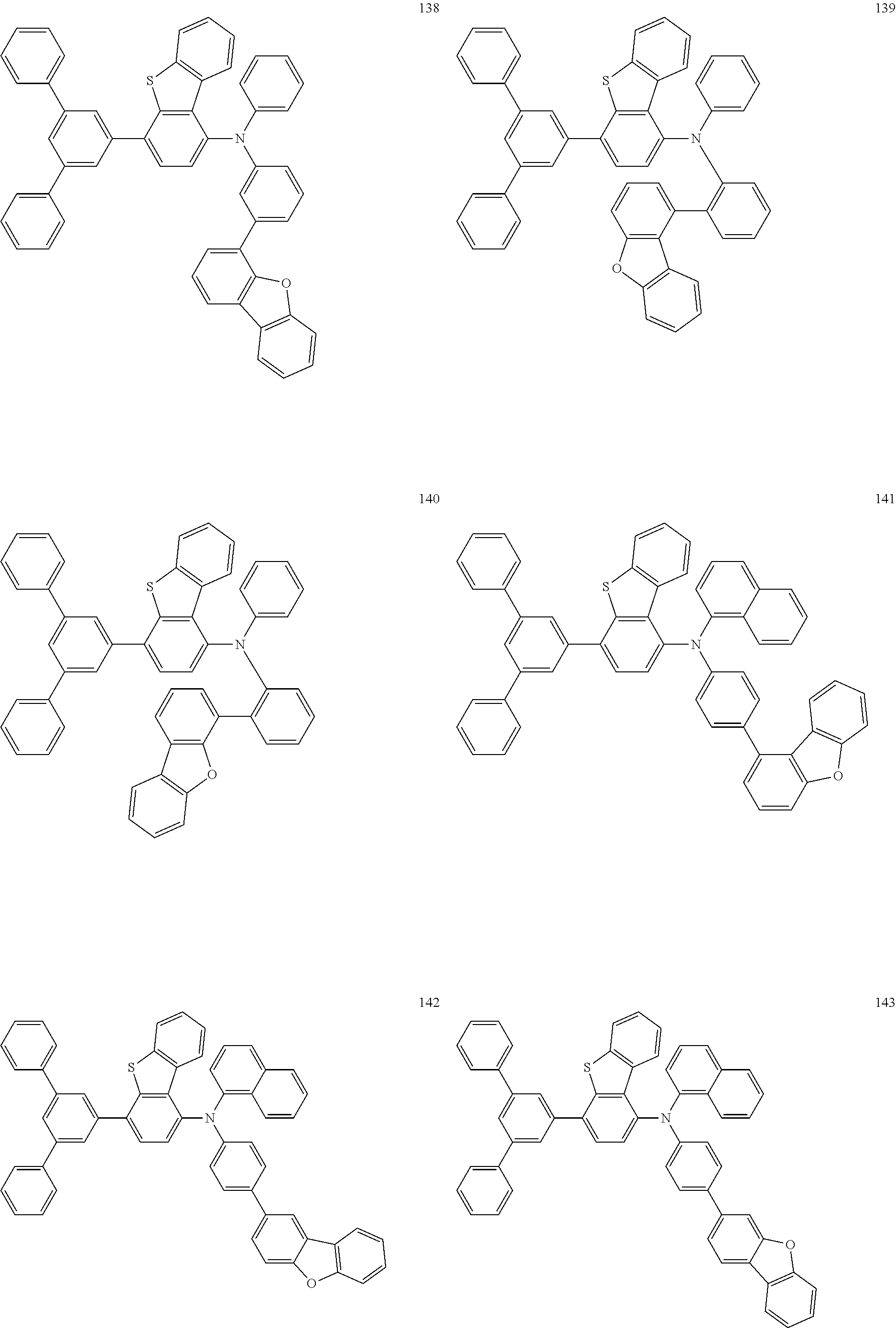

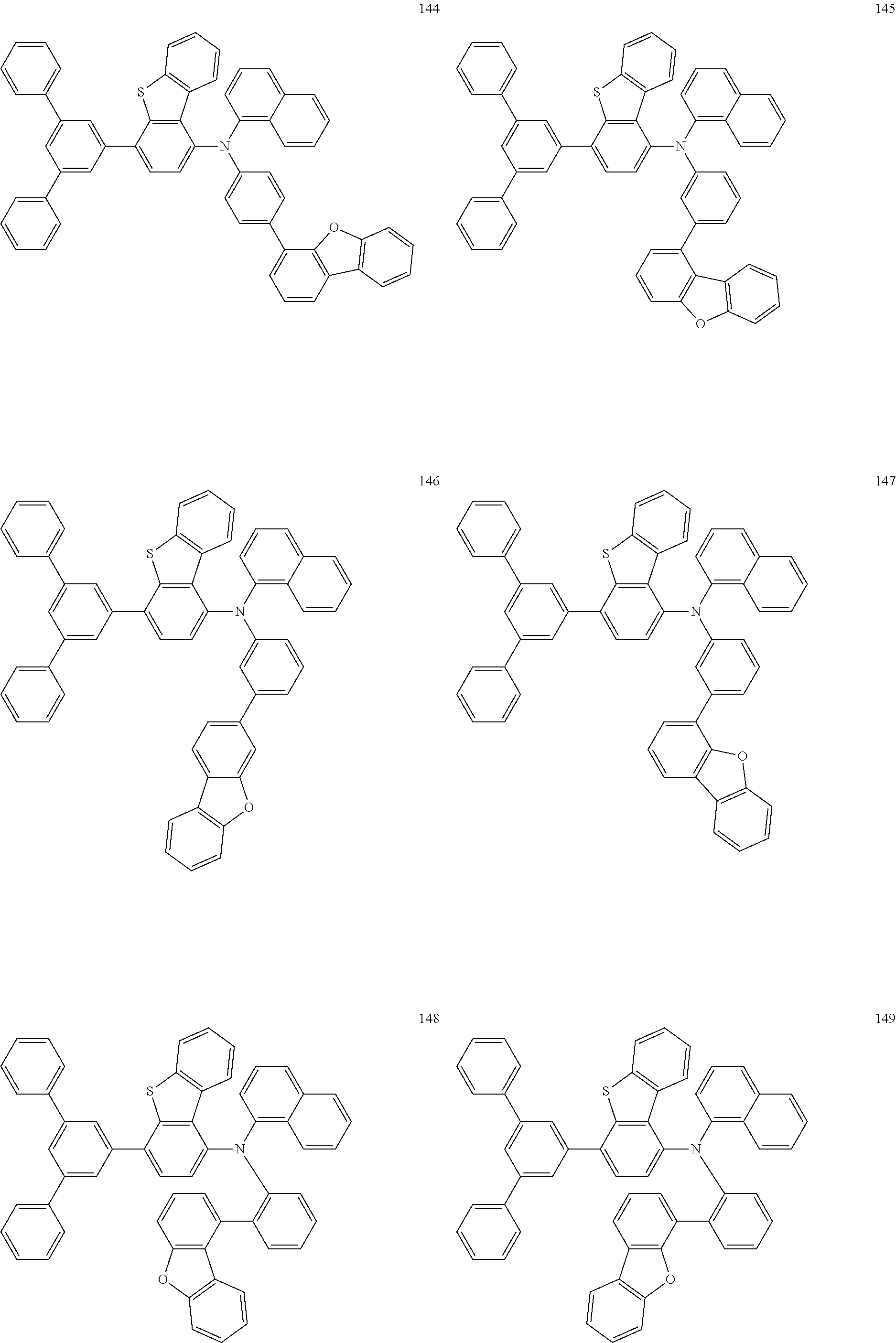

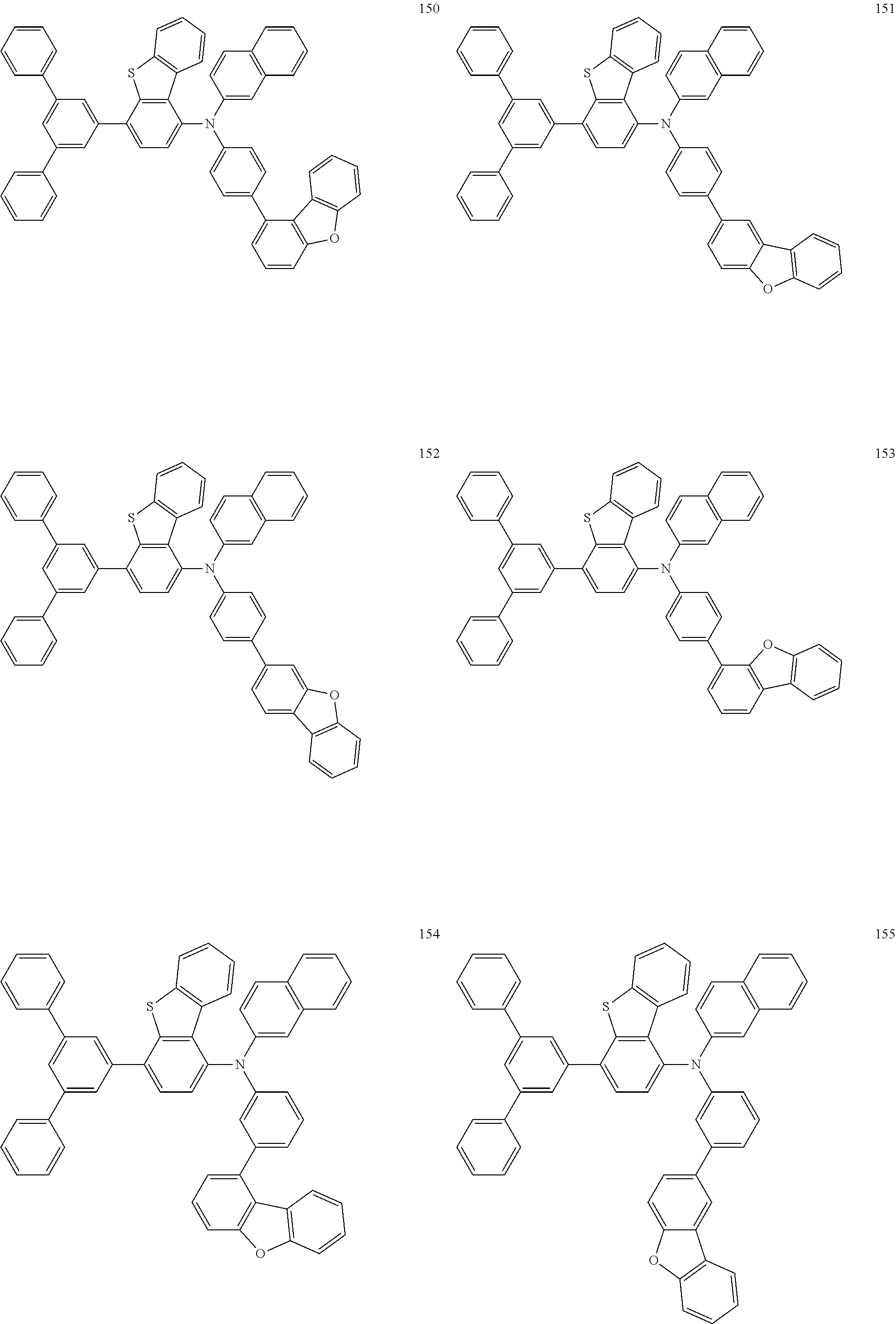

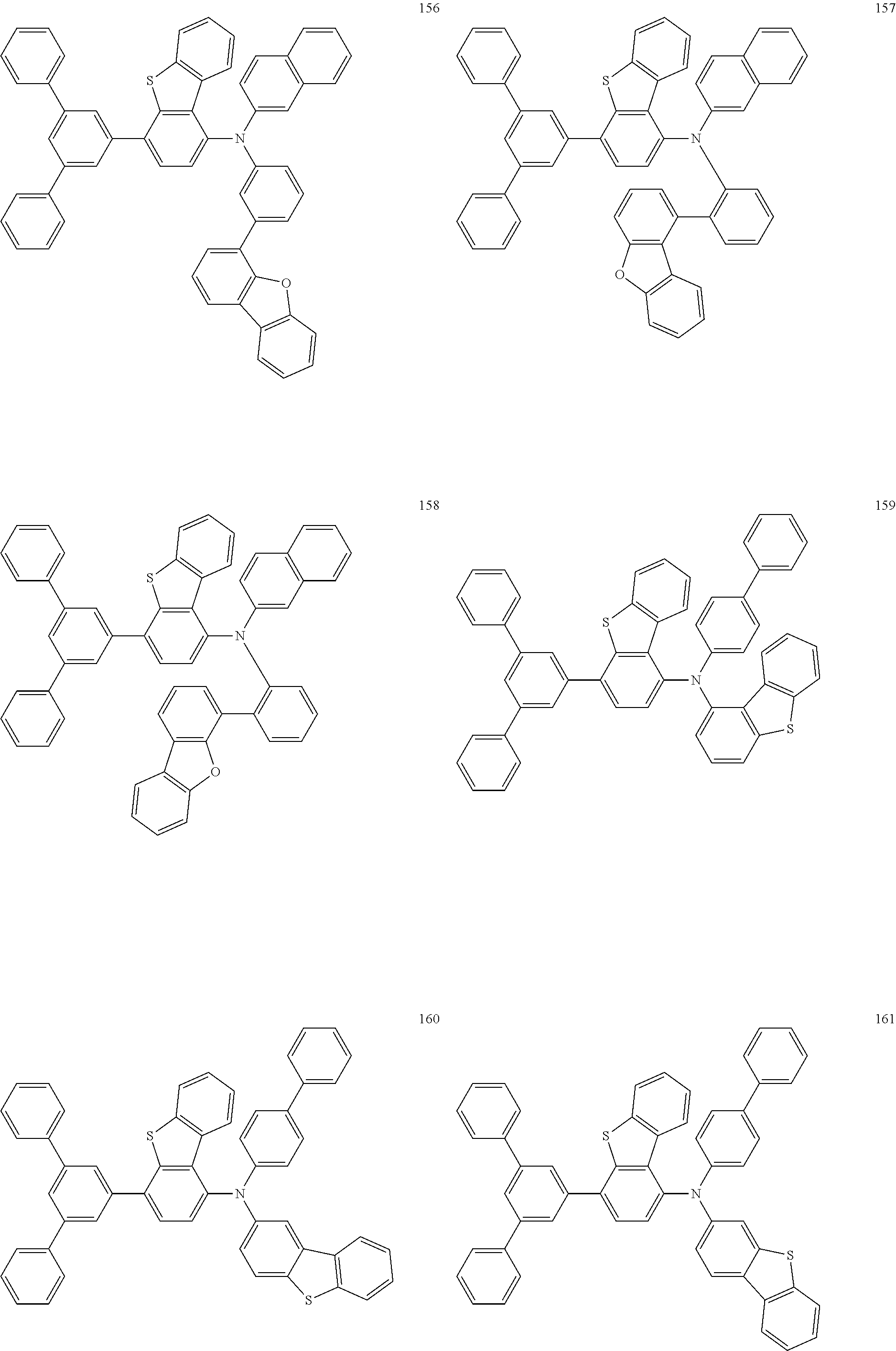

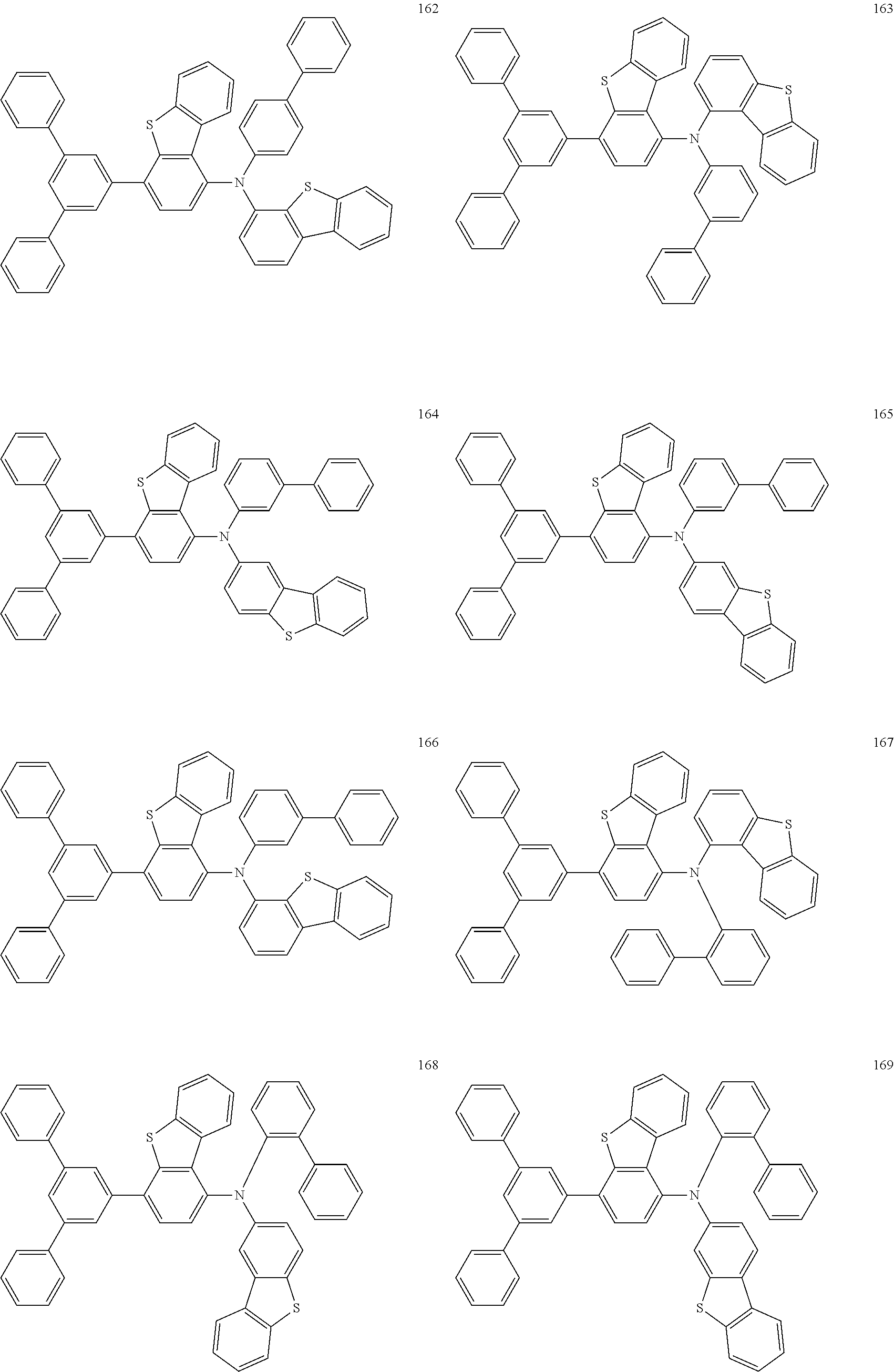

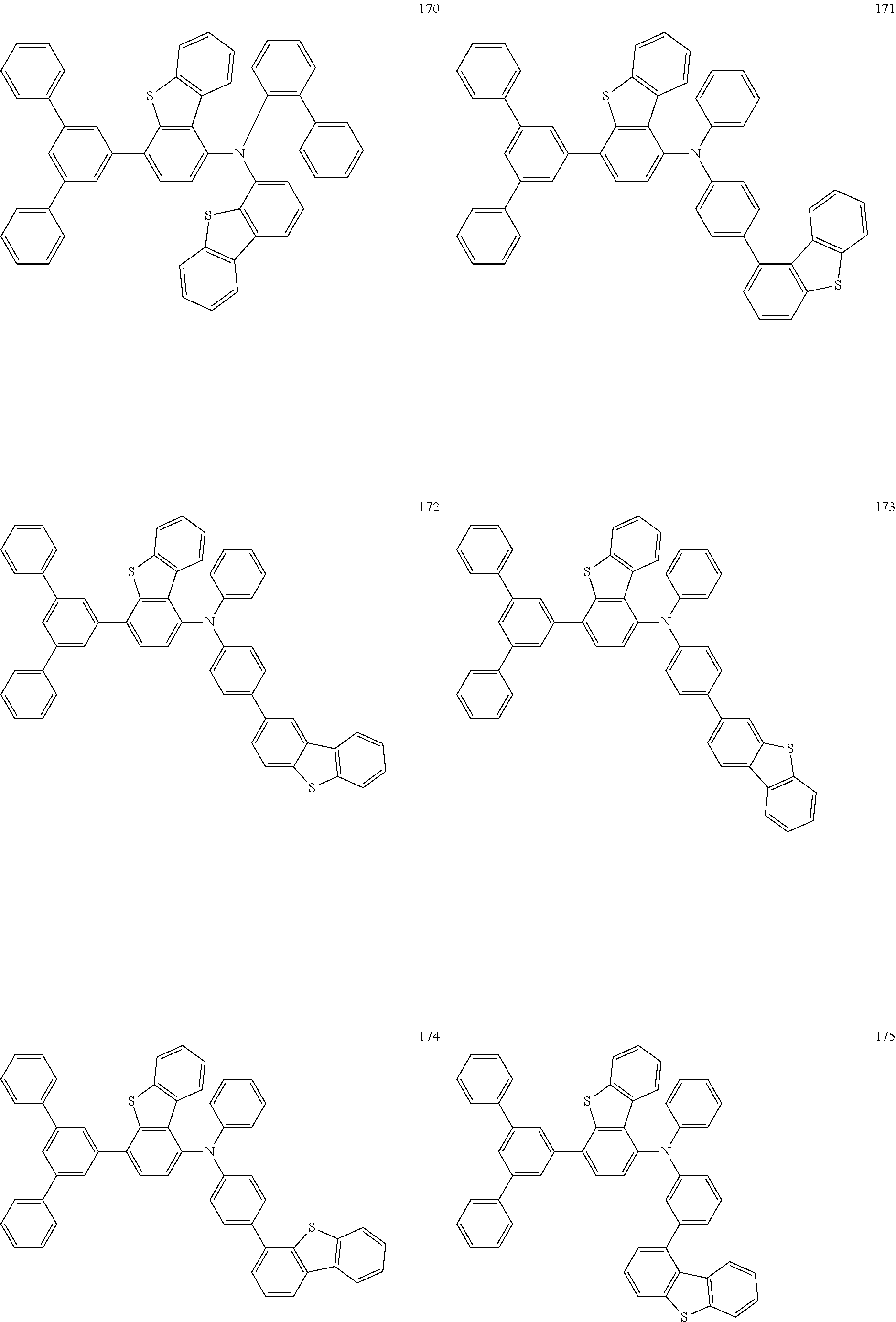

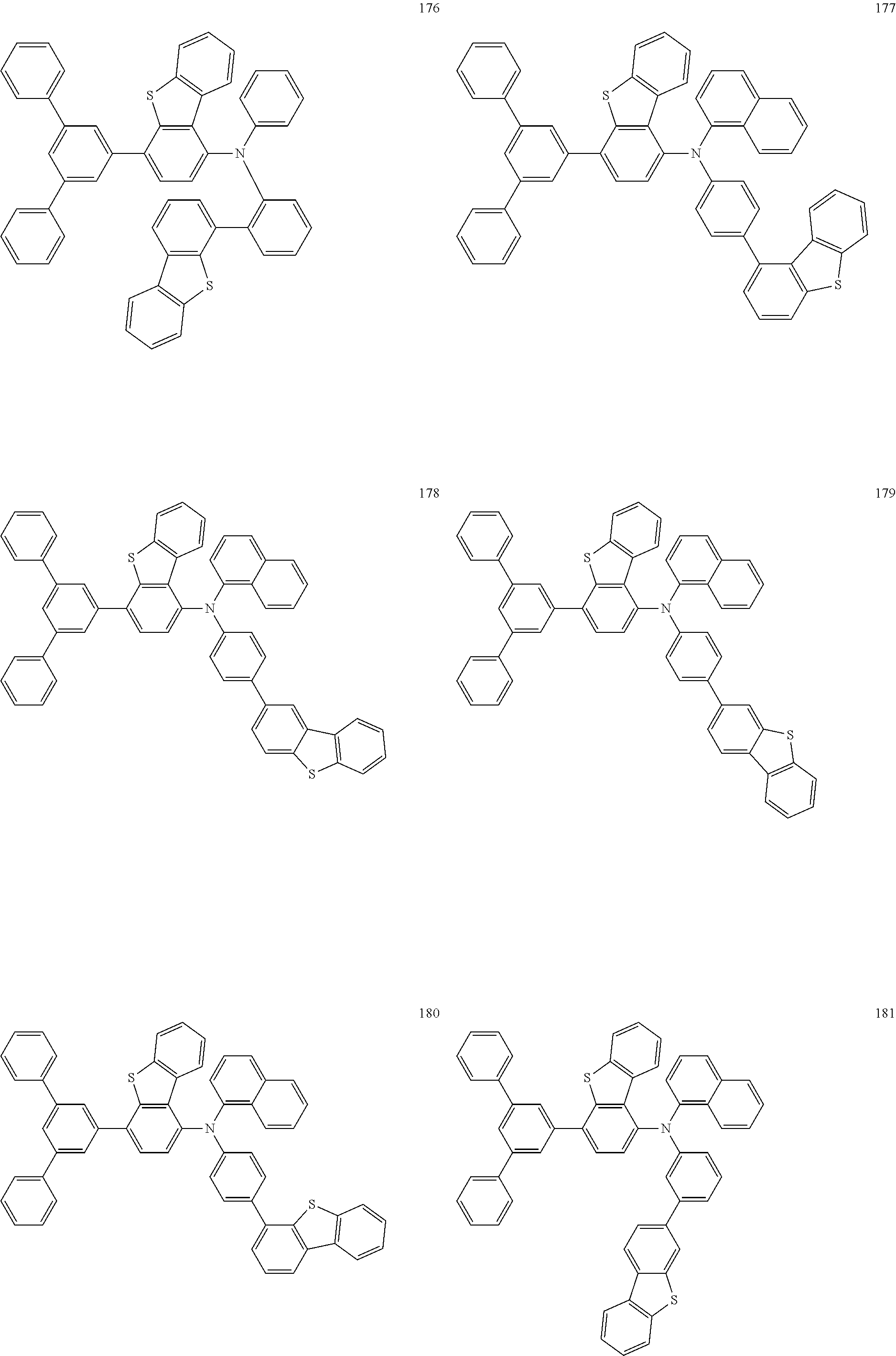

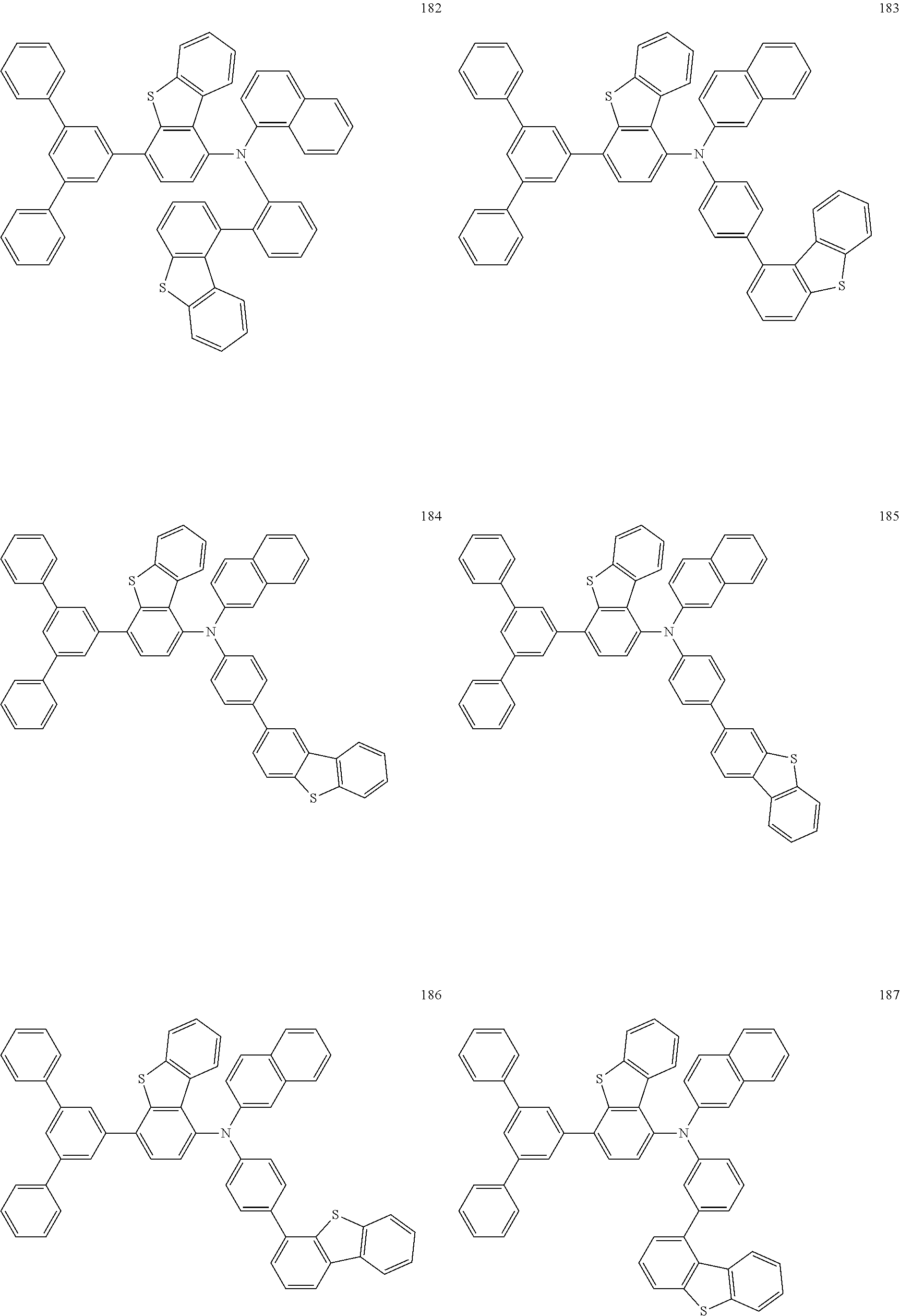

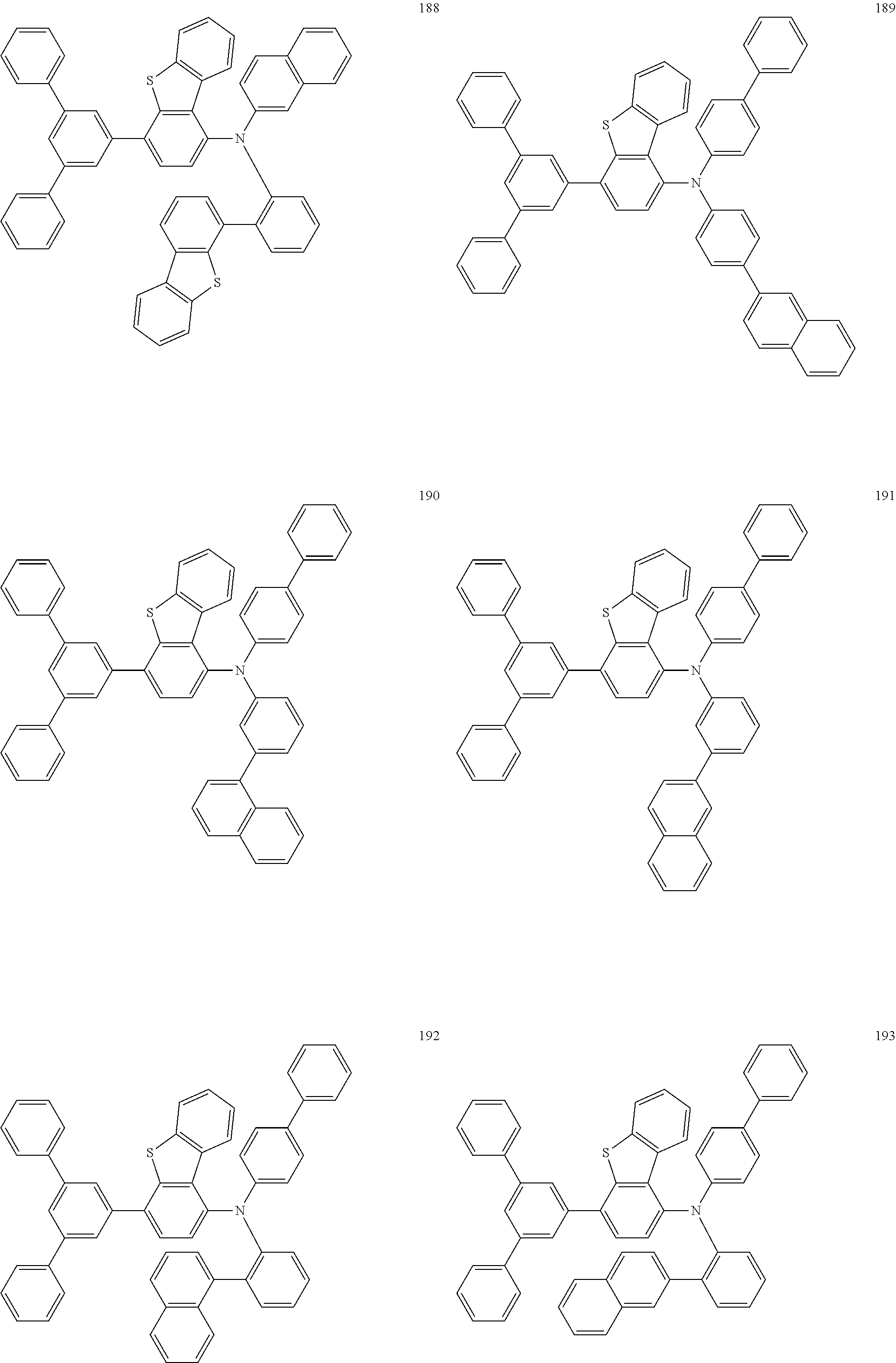

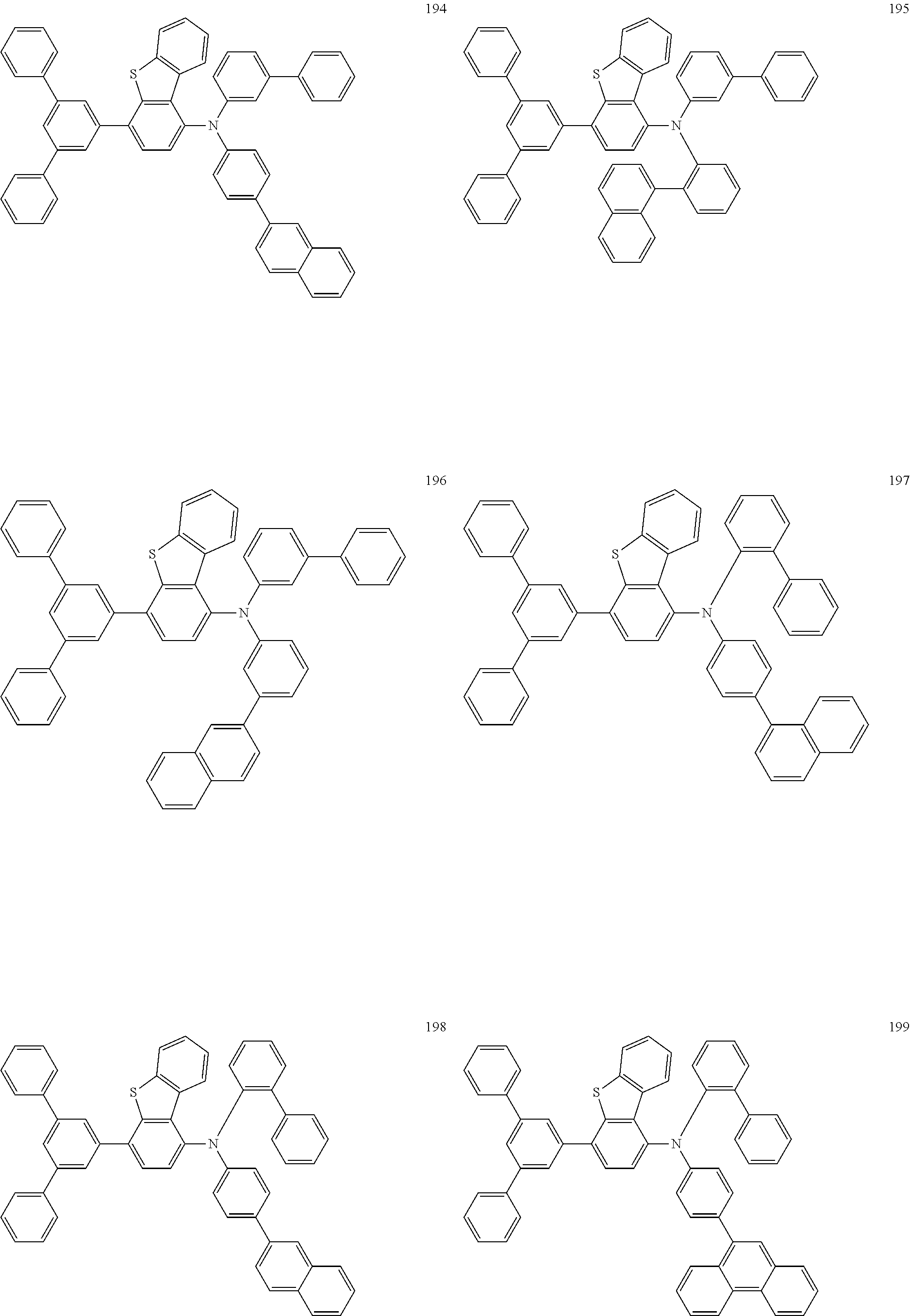

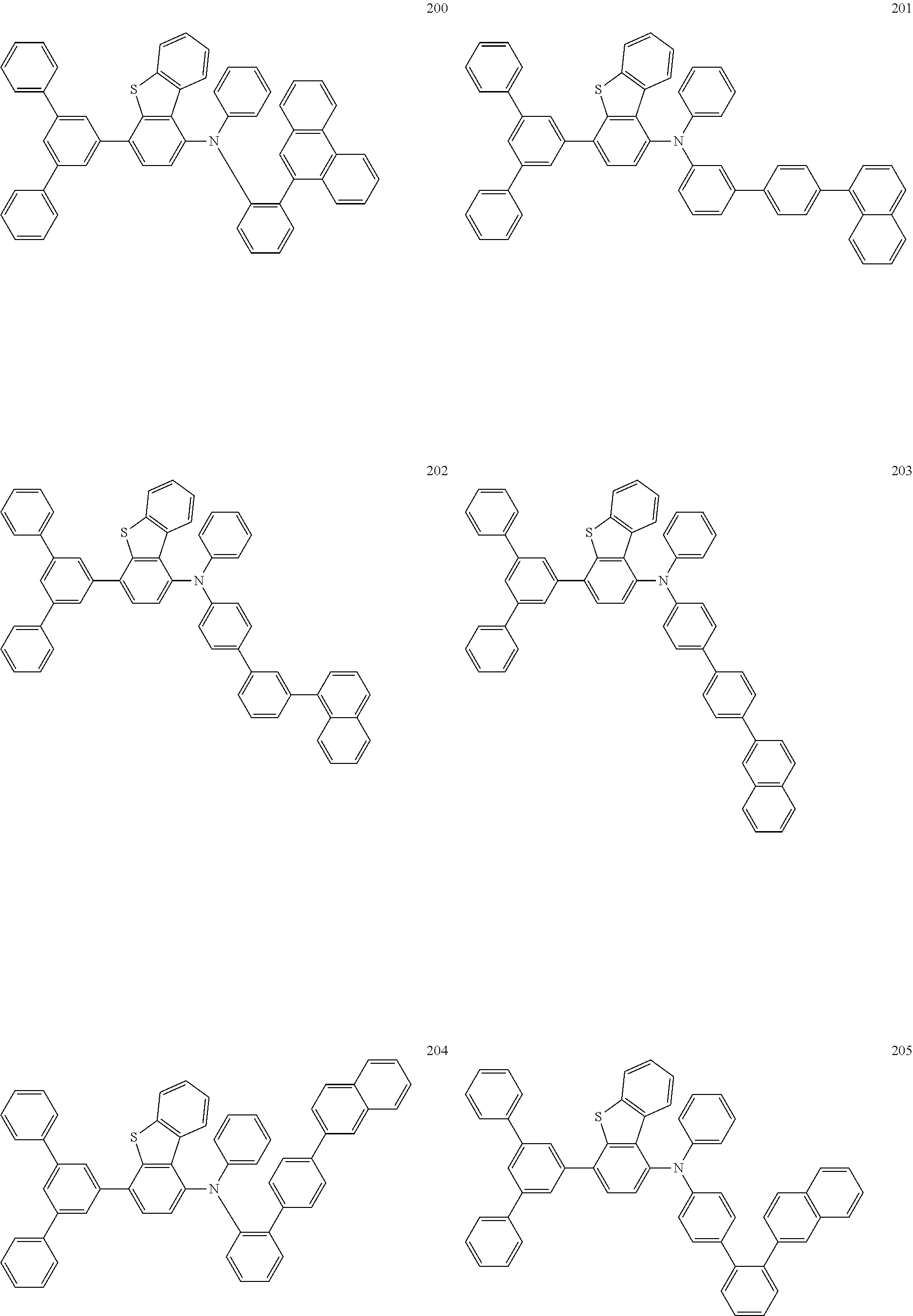

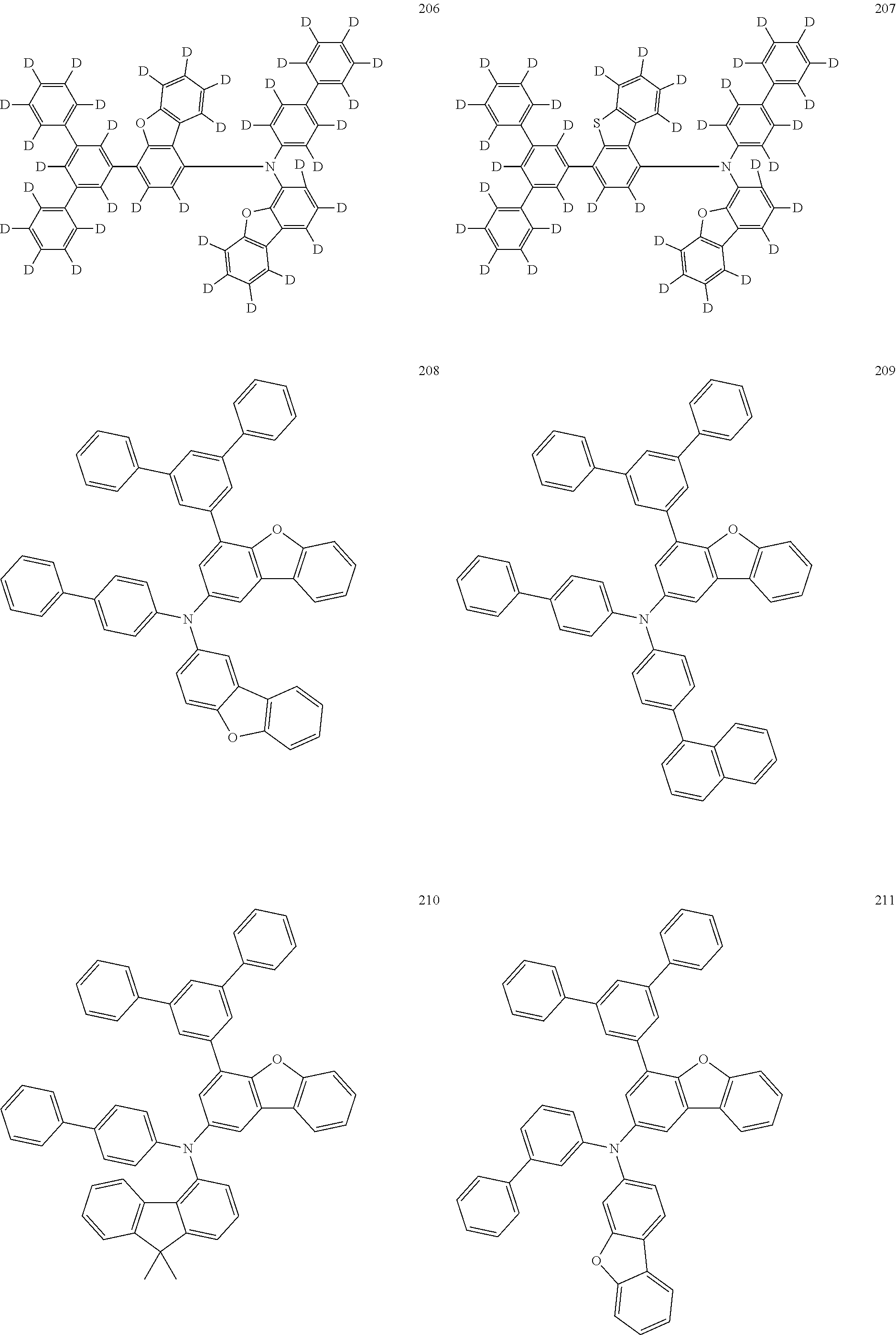

The organic compound represented by Chemical Formula A, of the present invention, is characterized in that an umbrella-shaped terphenyl group is substituted at a 4-position of dibenzofuran, and an amine group is included at a 1-position. The organic compound represented by Chemical Formula A, of the present invention, has a characteristic structure as described above, compared to a compound that does not satisfy a structure of Chemical Formula A, has an excellent hole injection characteristic. Therefore, when the organic compound represented by Chemical Formula A, of the present invention is applied to a hole transport layer and/or a hole transport auxiliary layer in an organic light-emitting diode, the organic compound may contribute to securing low operation voltage, high efficiency, and long lifetime characteristics in an element performance evaluation.

That is, when used as a material for the hole transport layer, the organic compound represented by Chemical Formula A, of the present invention may exhibit excellent hole mobility characteristics and thus may increase efficiency and lifetime of the element. In addition, when, in an embodiment, the organic compound represented by Chemical Formula A of the present invention is used as a material for the hole transport auxiliary layer, it is characterized in that the organic compound represented by Chemical Formula A exhibits excellent hole mobility and has an appropriate energy level as the hole transport auxiliary layer, which effectively transport holes to the emitting layer while blocking electrons migrating from the emitting layer, thereby improving efficiency and lifetime of the element.

In addition, the organic compound represented by Chemical Formula A of the present invention has a triplet energy level (T₁) within an appropriate range required for the hole transport layer and/or the hole transport auxiliary layer and thus may effectively suppress triplet excitons generated in the emitting layer from being diffused to outside, thereby improving light-emitting efficiency and element lifetime characteristics. In Particular, if the triplet energy level of the compound used for the hole transport layer and/or the hole transport auxiliary layer is excessively high, a HOMO energy level may also tend to become excessively deep, which may lead to a degradation in hole transport characteristics. Therefore, a lowered element performance such as an increase in the operation voltage may be caused. On the other hand, if the triplet energy level is low, it becomes impossible to prevent triplet excitons generated in the emitting layer from being diffused, and thus efficiency of the element may be lowered. The organic compound represented by Chemical Formula A of the present invention may have a triplet energy level within an appropriate range that secures a balance between a triplet exciton confinement effect and charge transport characteristics.

In addition, the organic compound represented by Chemical Formula A of the present invention may achieve low operation voltage, excellent current efficiency and lifetime characteristics, as well as excellent color reproduction when the organic compound represented by Chemical Formula A of the present invention is applied to the hole transport layer and/or the hole transport auxiliary layer.

More specifically, when the organic compound represented by Chemical Formula A of the present invention is used for the hole transport layer or the hole transport auxiliary layer, and is used for, for example, the hole transport auxiliary layer, the organic light-emitting diode may have improved efficiency and lifetime. In this case, unlike the organic compound represented by Chemical Formula A, according to the present invention, when a heteroaryl group or a fused polycyclic aryl group is not included, or when terphenyl is substituted at a 4-position and an amine group is not substituted at the 1-position of dibenzofuran, a deposition temperature required in a deposition process for forming the hole transport layer or the hole transport auxiliary layer may excessively increase. As a result, in addition to the inefficiency of the deposition process itself, a limitation such as degradation in thermal stability of a molecule may occur under the high-temperature conditions during the deposition process. In contrast, the organic compound represented by Chemical Formula A of the present invention may secure efficiency in the deposition process and excellent thermal stability of the molecule by substituting terphenyl and amine groups at specific positions of dibenzofuran and including a heteroaryl group or a fused polycyclic aryl group.

The organic light-emitting diode according to an embodiment of the inventive concept includes a first electrode (a positive electrode, anode), a second electrode (a negative electrode, cathode) facing the first electrode, and one or more organic material layers disposed between the first electrode and the second electrode, and at least one among the one or more organic material layers is a hole transport layer or hole transport auxiliary layer including the organic compound represented by Chemical Formula A above.

The organic material layer may include one or more among a hole injection layer (HIL), a hole transport layer (HTL), a hole transport auxiliary layer, an emitting layer (EML), an electron transport auxiliary layer, an electron transport layer (ETL), and an electron injection layer (EIL).

For example, the organic light-emitting diode may have a structure in which a first electrode, a hole injection layer (HIL), a hole transport layer (HTL), a hole transport auxiliary layer, an emitting layer (EML), an electron transport layer (ETL), an electron injection layer (EIL), and a second electrode are sequentially stacked.

Herein, the organic material layer including the organic compound represented by Chemical Formula A, according to an embodiment of the present invention, may be the hole transport layer (HTL), or the hole transport auxiliary layer.

The one or more organic material layers may further include one or more selected from the group consisting of a hole injection layer, an emitting layer, an electron transport auxiliary layer, an electron transport layer, and an electron injection layer.

The first electrode may be a positive electrode (anode), and the first electrode may include a transparent material with excellent conductivity, such as indium tin oxide (ITO), indium zinc oxide (IZO), tin oxide (SnO₂), and zinc oxide (ZnO).

The second electrode may be a negative electrode (cathode), and the second electrode may include a material such as lithium (Li), aluminum (Al), aluminum-lithium (AL-Li), calcium (Ca), magnesium (Mg), magnesium-indium (Mg—In), and magnesium-silver (Mg—Ag). In addition, a front emitting organic light-emitting diode may form a transparent second electrode which may transmit light using indium tin oxide (ITO), or indium zinc oxide (IZO).

A capping layer CPL may be formed on a surface of the second electrode by a composition for forming a capping layer.

In addition, an encapsulation layer or a protecting layer for protecting the organic light-emitting diode from moisture, oxygen, and the like may be additionally disposed on the capping layer. Such a protecting layer may be formed of a curable adhesive composition containing an inorganic desiccant.

The compound for a hole injection layer or hole transport layer is not particularly limited, and any compound commonly used as a material for a hole injection layer or a hole transport layer may be used. Non-limiting examples of the compound for hole injection layer or hole transport layer may include a phthalocyanine derivative, a porphyrin derivative, a triarylamine derivative, an indolocarbazole derivative, etc. For example, the hole injection layer or hole transport layer compound may include 1,4,5,8,9,11-hexaazatriphenylene-hexacarbonitrile (HAT-CN), copper phthalocyanine (CuPc), 4,4′,4″-tris(3-methylphenylamino)triphenyl amine (m-MTDATA), 4,4′,4″-tris(3-methylphenylamino) phenoxybenzene (m-MTDAPB), 4,4′,4″-tri(N-carbazolyl)triphenylamine (TCTA), 4,4′,4″-tris(N-(2-naphthyl)-N-phenylamino)-triphenylamine (2-TNATA), N4,N4,N4′,N4′-tetra([1,1′-biphenyl]-4-yl)-[1,1′-biphenyl]-4,4′-diamine, bis(N-(1-naphthyl-n-phenyl))benzidine (α-NPD), N,N′-di(naphthalen-1-yl)-N,N′-biphenyl-benzidine (NPB), N,N′-biphenyl-N,N′-bis(3-methylphenyl)-1,1′-biphenyl-4,4′-diamine (TPD), etc.

A compound included in the emitting layer is not specifically limited, and any compound may be used as long as it is commonly used as a compound for an emitting layer. A standalone light-emitting compound or a light-emitting host compound may be used.

The light-emitting compound for the emitting layer may include a compound that may emit light through phosphorescence, fluorescence, thermally activated delayed fluorescence (TADF, also referred to as E-type delayed fluorescence), triplet-triplet annihilation, or a combination of these processes but is not limited thereto. The light-emitting compound may be selected from various materials depending on desired emission colors. Non-limiting examples of the light-emitting compound may include a fused ring derivative such as phenanthrene, anthracene, pyrene, tetracene, pentacene, perylene, naphthopyrene, dibenzopyrene, rubrene, and chrysene, a benzoxazole derivative, a benzothiazole derivative, a benzimidazole derivative, a benzotriazole derivative, an oxazole derivative, an oxadiazole derivative, a thiazole derivative, an imidazole derivative, a thiadiazole derivative, a triazole derivative, a pyrazoline derivative, a stilbene derivative, a thiophene derivative, a tetraphenylbutadiene derivative, a cyclopentadiene derivative, a bis-styryl derivative, a bis-styrylarylrene derivative, a diazaindacene derivative, a furan derivative, a benzofuran derivative, an isobenzofuran derivative, a dibenzofuran derivative, a coumarin derivative, a diacynomethylenepyran derivative, a diacynomethylenethiopyran derivative, a polymethine derivative, a cyanine derivative, an oxo-benzanthracene derivative, a xanthene derivative, a rhodamine derivative, a fluorescein derivative, a pyrylium derivative, a carbostyryl derivative, an acridine derivative, an oxazine derivative, a phenylene oxide derivative, a quinacridone derivative, a quinazoline derivative, a pyrrolopyridine derivative, a furopyridine derivative, an 1,2,5-thiadia zolopyrene derivative, a pyrromethene derivative, a perinone derivative, a pyrrolopyrrol derivative, a squarylium derivative, a violanthrone derivative, a phenazine derivative, an acridone derivative, a deazaflavin derivative, a fluorene derivative, a benzofluorene derivative, an aromatic boron derivative, an aromatic nitrogen boron derivative, a metal complex (a bonded complex of a metal such as Ir, Pt, Au, Eu, Ru, Re, Ag, and Cu, and a heteroaromatic ring ligand), etc. For example, N1,N1,N6,N6-tetrakis(4-(1-silyl)phenyl) pyrene-1,6-diamine, 2,12-di-tert-butyl-5,9-bis(4-(tert-butyl)phenyl)-7-(3,5-di-tert-butylphenyl)-5,9-dihydro-5,9-diaza-13b-borannaphtho[3,2,1-de]anthracene (t-DABNA-dtB), platinumoctaethylporphyrin (PtOEP), Ir(ppy)₃, Ir(ppy)₂(acac), Ir(mppy)₃, Ir(PPy)₂ (m-bppy), BtpIr(acac), Ir(btp)₂(acac), Ir(2-phq)₃, Hex-Ir(phq)₃, Ir(fbi)₂(acac), fac-Tris(2-(3-p-xylyl)phenyl)pyridine iridium (III), Eu(dbm)₃(Phen), Ir(piq)₃, Ir(piq)₂(acac), Ir(Fliq)₂(acac), Ir(Flq)₂(acac), Ru(dtb-bpy)₃·2(PF₆), Ir(BT)₂(acac), Ir(DMP)₃, Ir(Mphq)₃IR(phq)₂tpy, fac-Ir(ppy)₂Pc, Ir(dp)PQ₂, Ir(Dpm)(Piq)₂, Hex-Ir(piq)₂(acac), Ir(dmpq)₃, Ir(dmpq)₂(acac), FPQIrpic, FIrpic, etc., may be included.

As a host material of the emitting layer, a light-emitting host, a hole transporting host, an electron transporting host, or a combination thereof may be used. Non-limiting examples of the light-emitting host material may include a fused ring derivative such as anthracene, or pyrene, a bis-styryl derivative such as a bis-styrylanthracene derivative, or a di-styrylbenzene derivative, a tetraphenylbutadiene derivative, a cyclopentadiene derivative, a fluorene derivative, a benzofluorene derivative, a n-phenylcarbazole (9-phenylcarbazole) derivative, a carbazolylnitrile derivative, etc. Non-limiting examples of the hole-transporting host material may include a carbazole derivative, a dibenzofuran derivative, a dibenzothiophene derivative, a triarylamine derivative, an indolocarbazole derivative, and a benzoxazinophenoxazine derivative. Non-limiting examples of the electron-transporting host material may include a pyridine derivative, a triazine derivative, a phosphine oxide derivative, a benzofuropyridine derivative, and a dibenzoxasiline derivative. For example, non-limiting examples of the electron-transporting host material may include 9,10-bis(2-naphthyl) anthracene (ADN), tris(8-hydroxyquinolinato)aluminum (Alq₃), bis(8-hydroxyquinolinato) beryllium (BAlq), 4,4′-bis(2,2-biphenylethenyl)-1,1′-biphenyl series (DPVBi), spiro-4,4′-bis(2,2-biphenylethenyl)-1,1′-biphenyl(spiro-DPVBi), (2-(2-benzoxazolyl)-phenolato) lithium (LiPBO), bis(biphenylvinyl)benzene, an aluminum-quinoline metal complex, a metal complex of imidazole, thiazole, or oxazole, etc.

The electron injection layer or electron transport layer compound is not specifically limited, and any compound that is commonly used as an electron injection layer or electron transport layer compound may be used. Non-limiting examples of the electron injection layer or electron transport layer compound may include a pyridine derivative, a naphthalene derivative, an anthracene derivative, a phenanthroline derivative, a perinone derivative, a coumarin derivative, a naphthalimide derivative, an anthraquinone derivative, a diphenoquinone derivative, a diphenylquinone derivative, a perylene derivative, an oxadiazole derivative, a thiophene derivative, a triazole derivative, a thiadiazole derivative, a metal complex of an oxine derivative, a quinolinol-based metal complex, a quinoxaline derivative, a polymer of a quinoxaline derivative, a benzazole-based compound, a gallium complex, a pyrazole derivative, a perfluorinated phenylene derivative, a triazine derivative, a pyrazine derivative, a benzoquinoline derivative, an imidazopyridine derivative, a borane derivative, a benzimidazole derivative, a benzoxazole derivative, a benzothiazole derivative, a quinoline derivative, an oligo-pyridine derivative such as terpyridine, a bipyridine derivative, a terpyridine derivative, a naphthyridine derivative, an aldazine derivative, a carbazole derivative, an indole derivative, a phosphine oxide derivative, a bis-styryl derivative, a quinolinol-based metal complex, a hydroxyazole-based metal complex, an azomethine-based metal complex, a tropolone-based metal complex, a flavonol-based metal complex, a benzoquinoline-based metal complex, a metal salt, etc. These materials may be used independently or as a mixture with another material. For example, a material such as 2-(4-(9,10-di(naphthalene-2-yl) anthracene-2-yl)phenyl)-1-phenyl-1H-benzo[d]imidazole, tris(8-hydroxyquinolinato)aluminum (Alq₃), LiF, Liq, Li₂O, BaO, NaCl, and CsF may be included.

A compound for an electron transport auxiliary layer disposed between the electron transport layer and the emitting layer is not specifically limited, and any compound may be used as long as it is commonly used as a compound for the electron transport auxiliary layer. For example, the electron transport auxiliary layer may include a pyrimidine derivative, and the like.

The organic light-emitting diode according to an embodiment of the present invention may be a top-emission type, or a bottom-emission type.

The organic light-emitting diode according to an embodiment of the present invention may be used in a display device.

The organic light-emitting diode according to an embodiment of the present invention may be applied to a transparent display device, a mobile display device, a flexible display device, and the like, but is not limited thereto.

The organic light-emitting diode according to an embodiment of the present invention may have a tandem structure including a plurality light-emitting stacks between the positive electrode and the negative electrode.

Hereinafter, synthesis methods of the compounds will be described below by exemplifying representative examples. However, the synthesis methods of the compounds according to the present invention are not limited to the following exemplified methods.

### SYNTHESIS EXAMPLES

A synthesis Example of Compound 1 is described as a representative example, and the compounds of Chemical Formula A of the present invention may be synthesized in a similar manner to a reaction of Compound 1.

In the following reaction scheme, a solvent, a catalyst, a protecting group, a leaving group, a reaction temperature, a reaction time, and the like are provided as representative examples, and all equivalents in terms of a solvent, a catalyst, a protecting group, a leaving group, a reaction temperature, a reaction time, equivalents of a reactant, and the like may be adopted.

### 1. Synthesis of Compound 1

Compound 1 may be synthesized as follows, but an embodiment of the present invention is not limited thereto.

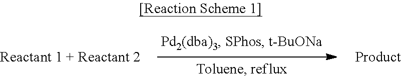

Under a nitrogen atmosphere, Reactant 1 of Compound 1 (35 mmol), Reactant 2 of Compound 1 (36 mmol), Pd2 (dba) 3 (0.5 mmol), SPhos (1 mmol), t-BuONa (52 mmol), and toluene (150 mL) were added to a reaction flask and were stirred and refluxed. An organic layer was extracted using toluene, and water. The extracted solution was treated with MgSO₄ to remove residual moisture, extracted under a reduced pressure, and then purified and then recrystallized using a column chromatography method to obtain a product. The synthesis results of Compound 1 are as listed in Table 1 below.

The synthesized compounds are representatively shown in Table 1 below, and the specific compounds of the present invention and compounds similar thereto may be synthesized through the above synthetic examples.

TABLE 1

| | | | | Amount | |
|---|---|---|---|---|---|
| | | | | of | |
| Com- | | | | product | [M + |
| pound | Reactant 1 | Reactant 2 | Product | (Yield) | H]⁺ |
| 1 | 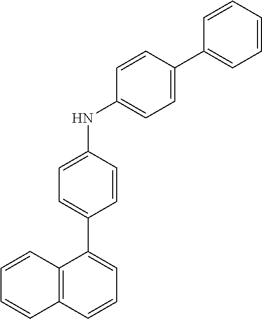 [SMILES: c1ccc(-c2ccc(Nc3ccc(-c4cccc5ccccc45)cc3)cc2)cc1] | 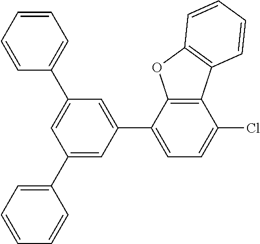 [SMILES: Clc1ccc(-c2cc(-c3ccccc3)cc(-c3ccccc3)c2)c2oc3ccccc3c12] | 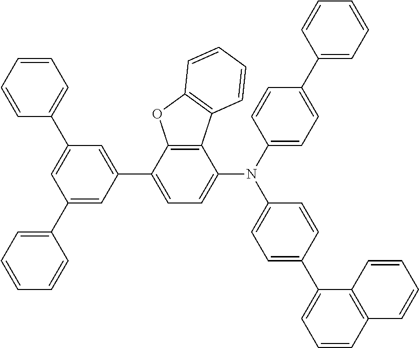 [SMILES: c1ccc(-c2ccc(N(c3ccc(-c4cccc5ccccc45)cc3)c3ccc(-c4cc(-c5ccccc5)cc(-c5ccccc5)c4)c4oc5ccccc5c34)cc2)cc1] | 22.5 g (84%) | 765.30 |
| 2 | 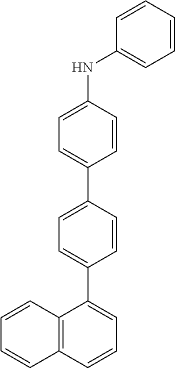 [SMILES: c1ccc(Nc2ccc(-c3ccc(-c4cccc5ccccc45)cc3)cc2)cc1] | 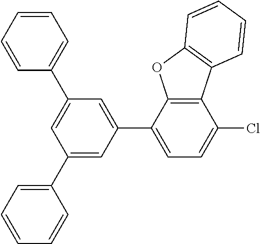 [SMILES: Clc1ccc(-c2cc(-c3ccccc3)cc(-c3ccccc3)c2)c2oc3ccccc3c12] | 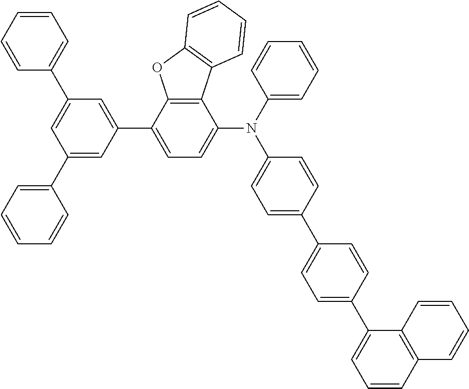 [SMILES: c1ccc(-c2cc(-c3ccccc3)cc(-c3ccc(N(c4ccccc4)c4ccc(-c5ccc(-c6cccc7ccccc67)cc5)cc4)c4c3oc3ccccc34)c2)cc1] | 21.2 g (79%) | 765.30 |
| 3 | 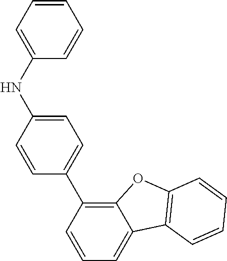 [SMILES: c1ccc(Nc2ccc(-c3cccc4c3oc3ccccc34)cc2)cc1] | 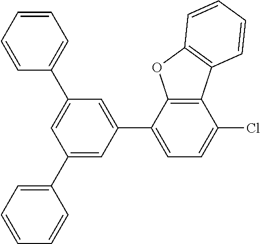 [SMILES: Clc1ccc(-c2cc(-c3ccccc3)cc(-c3ccccc3)c2)c2oc3ccccc3c12] | 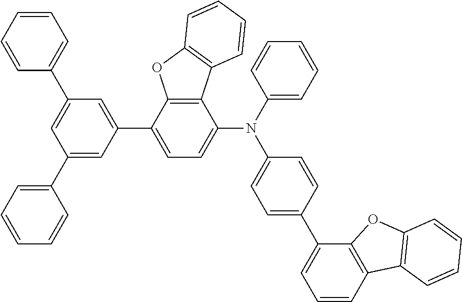 [SMILES: c1ccc(-c2cc(-c3ccccc3)cc(-c3ccc(N(c4ccccc4)c4ccc(-c5cccc6c5oc5ccccc56)cc4)c4c3oc3ccccc34)c2)cc1] | 20.7 g (81%) | 729.27 |
| 4 | 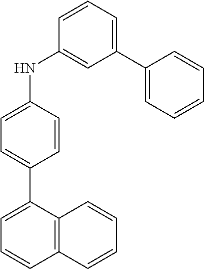 [SMILES: c1ccc(-c2cccc(Nc3ccc(-c4cccc5ccccc45)cc3)c2)cc1] | 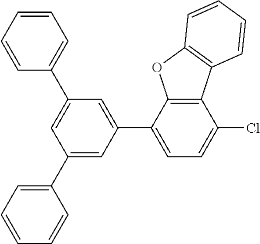 [SMILES: Clc1ccc(-c2cc(-c3ccccc3)cc(-c3ccccc3)c2)c2oc3ccccc3c12] | 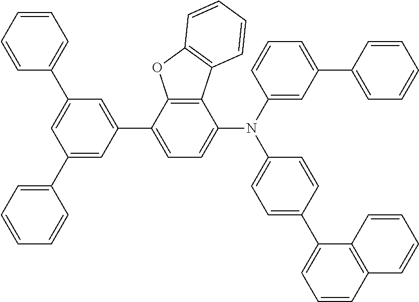 [SMILES: c1ccc(-c2cc(-c3ccccc3)cc(-c3ccc(N(c4ccc(-c5cccc6ccccc56)cc4)c4cccc(-c5ccccc5)c4)c4c3oc3ccccc34)c2)cc1] | 22.0 g (82%) | 765.30 |
| 5 | 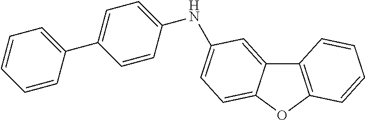 [SMILES: c1ccc(-c2ccc(Nc3ccc4oc5ccccc5c4c3)cc2)cc1] | 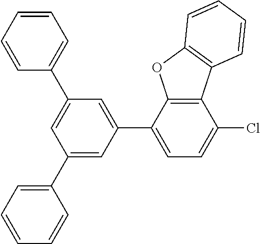 [SMILES: Clc1ccc(-c2cc(-c3ccccc3)cc(-c3ccccc3)c2)c2oc3ccccc3c12] | 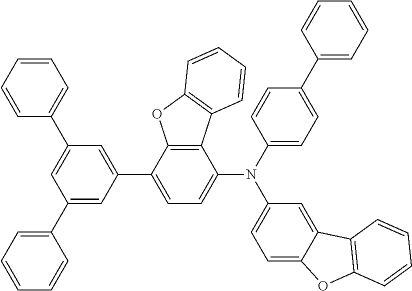 [SMILES: c1ccc(-c2ccc(N(c3ccc4oc5ccccc5c4c3)c3ccc(-c4cc(-c5ccccc5)cc(-c5ccccc5)c4)c4oc5ccccc5c34)cc2)cc1] | 20.4 g (80%) | 729.27 |
| 6 | 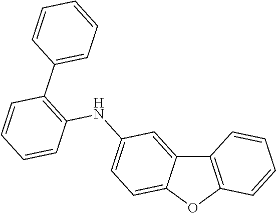 [SMILES: c1ccc(-c2ccccc2Nc2ccc3oc4ccccc4c3c2)cc1] | 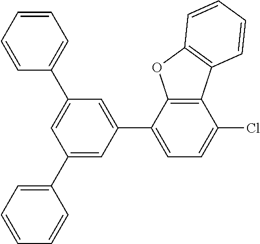 [SMILES: Clc1ccc(-c2cc(-c3ccccc3)cc(-c3ccccc3)c2)c2oc3ccccc3c12] | 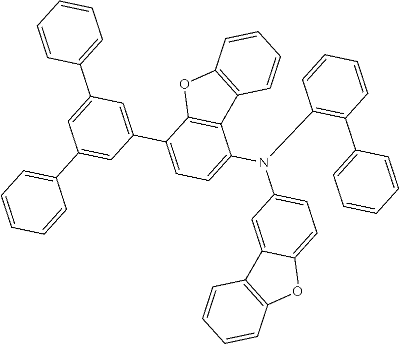 [SMILES: c1ccc(-c2cc(-c3ccccc3)cc(-c3ccc(N(c4ccc5oc6ccccc6c5c4)c4ccccc4-c4ccccc4)c4c3oc3ccccc34)c2)cc1] | 20.2 g (79%) | 729.27 |
| 27 | 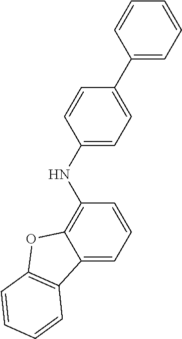 [SMILES: c1ccc(-c2ccc(Nc3cccc4c3oc3ccccc34)cc2)cc1] | 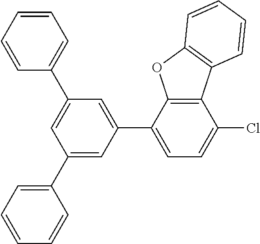 [SMILES: Clc1ccc(-c2cc(-c3ccccc3)cc(-c3ccccc3)c2)c2oc3ccccc3c12] | 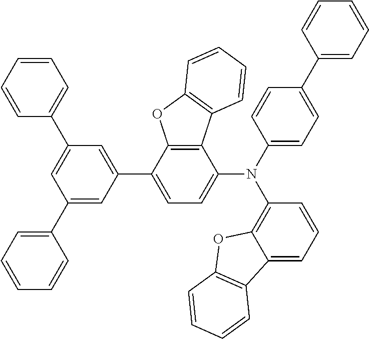 [SMILES: c1ccc(-c2ccc(N(c3cccc4c3oc3ccccc34)c3ccc(-c4cc(-c5ccccc5)cc(-c5ccccc5)c4)c4oc5ccccc5c34)cc2)cc1] | 20.7 g (81%) | 729.27 |
| 98 | 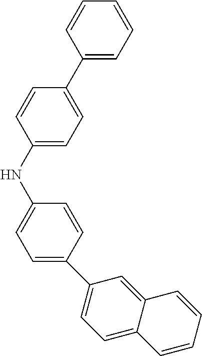 [SMILES: c1ccc(-c2ccc(Nc3ccc(-c4ccc5ccccc5c4)cc3)cc2)cc1] | 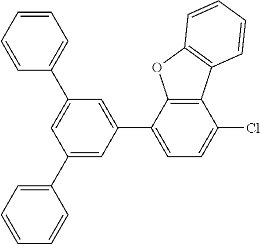 [SMILES: Clc1ccc(-c2cc(-c3ccccc3)cc(-c3ccccc3)c2)c2oc3ccccc3c12] | 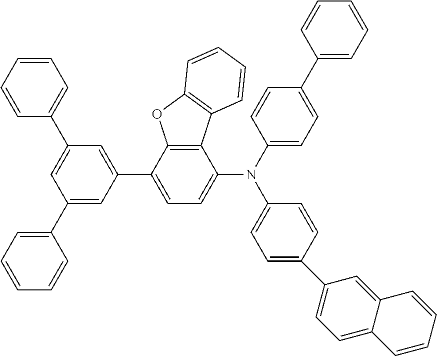 [SMILES: c1ccc(-c2ccc(N(c3ccc(-c4ccc5ccccc5c4)cc3)c3ccc(-c4cc(-c5ccccc5)cc(-c5ccccc5)c4)c4oc5ccccc5c34)cc2)cc1] | 21.4 g (80%) | 765.30 |
| 45 | 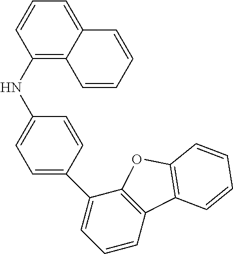 [SMILES: c1ccc2c(Nc3ccc(-c4cccc5c4oc4ccccc45)cc3)cccc2c1] | 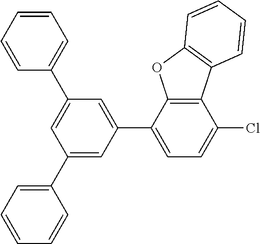 [SMILES: Clc1ccc(-c2cc(-c3ccccc3)cc(-c3ccccc3)c2)c2oc3ccccc3c12] | 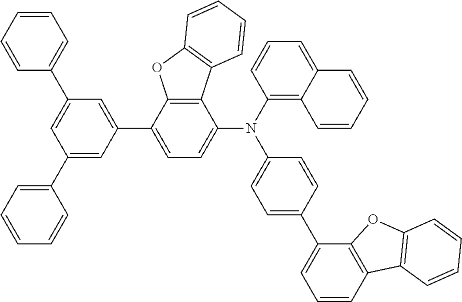 [SMILES: c1ccc(-c2cc(-c3ccccc3)cc(-c3ccc(N(c4ccc(-c5cccc6c5oc5ccccc56)cc4)c4cccc5ccccc45)c4c3oc3ccccc34)c2)cc1] | 21.3 g (78%) | 779.28 |
| 35 | 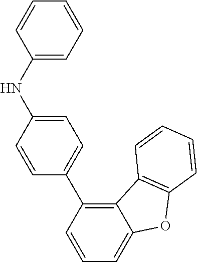 [SMILES: c1ccc(Nc2ccc(-c3cccc4oc5ccccc5c34)cc2)cc1] | 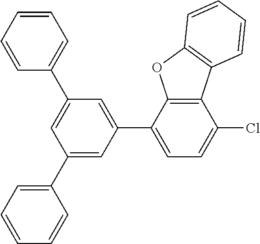 [SMILES: Clc1ccc(-c2cc(-c3ccccc3)cc(-c3ccccc3)c2)c2oc3ccccc3c12] | 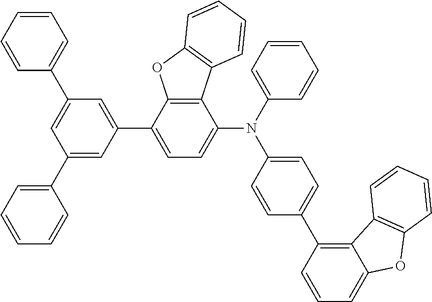 [SMILES: c1ccc(-c2cc(-c3ccccc3)cc(-c3ccc(N(c4ccccc4)c4ccc(-c5cccc6oc7ccccc7c56)cc4)c4c3oc3ccccc34)c2)cc1] | 20.7 g (81%) | 729.27 |
| 108 | 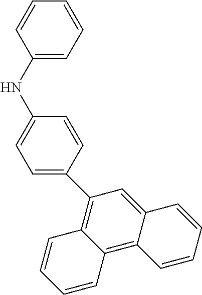 [SMILES: c1ccc(Nc2ccc(-c3cc4ccccc4c4ccccc34)cc2)cc1] | 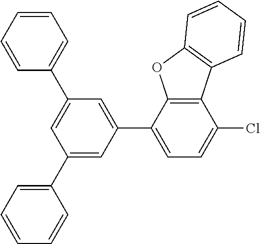 [SMILES: Clc1ccc(-c2cc(-c3ccccc3)cc(-c3ccccc3)c2)c2oc3ccccc3c12] | 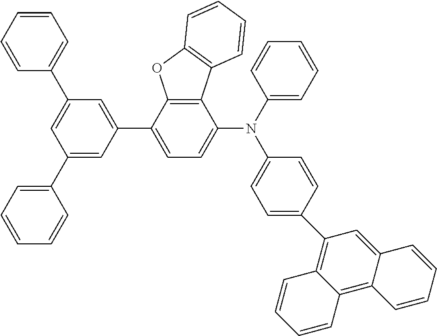 [SMILES: c1ccc(-c2cc(-c3ccccc3)cc(-c3ccc(N(c4ccccc4)c4ccc(-c5cc6ccccc6c6ccccc56)cc4)c4c3oc3ccccc34)c2)cc1] | 21.2 g (82%) | 739.29 |
| 34 | 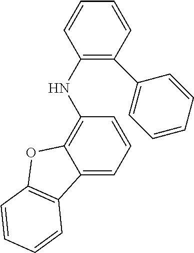 [SMILES: c1ccc(-c2ccccc2Nc2cccc3c2oc2ccccc23)cc1] | 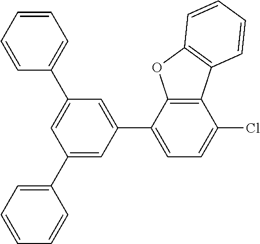 [SMILES: Clc1ccc(-c2cc(-c3ccccc3)cc(-c3ccccc3)c2)c2oc3ccccc3c12] | 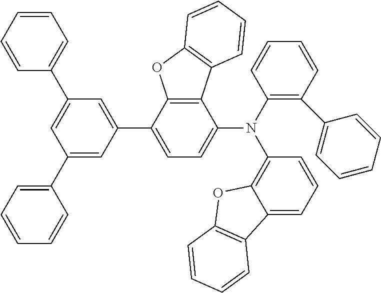 [SMILES: c1ccc(-c2cc(-c3ccccc3)cc(-c3ccc(N(c4ccccc4-c4ccccc4)c4cccc5c4oc4ccccc45)c4c3oc3ccccc34)c2)cc1] | 19.9 g (78%) | 729.27 |
| 28 | 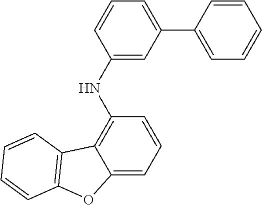 [SMILES: c1ccc(-c2cccc(Nc3cccc4oc5ccccc5c34)c2)cc1] | 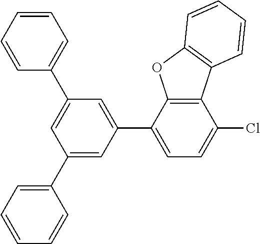 [SMILES: Clc1ccc(-c2cc(-c3ccccc3)cc(-c3ccccc3)c2)c2oc3ccccc3c12] | 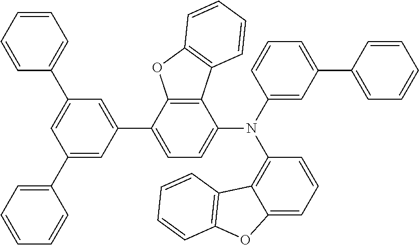 [SMILES: c1ccc(-c2cc(-c3ccccc3)cc(-c3ccc(N(c4cccc(-c5ccccc5)c4)c4cccc5oc6ccccc6c45)c4c3oc3ccccc34)c2)cc1] | 20.2 g (79%) | 729.27 |
| 31 | 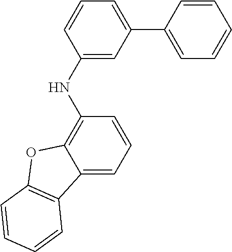 [SMILES: c1ccc(-c2cccc(Nc3cccc4c3oc3ccccc34)c2)cc1] | 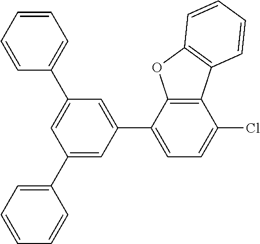 [SMILES: Clc1ccc(-c2cc(-c3ccccc3)cc(-c3ccccc3)c2)c2oc3ccccc3c12] | 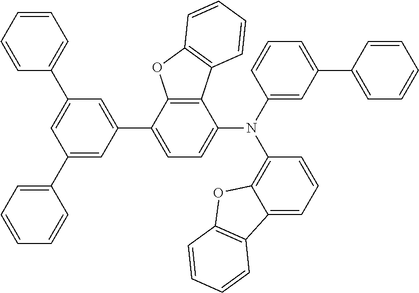 [SMILES: c1ccc(-c2cc(-c3ccccc3)cc(-c3ccc(N(c4cccc(-c5ccccc5)c4)c4cccc5c4oc4ccccc45)c4c3oc3ccccc34)c2)cc1] | 20.4 g (80%) | 729.27 |
| 25 | 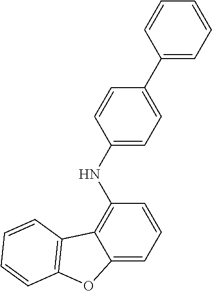 [SMILES: c1ccc(-c2ccc(Nc3cccc4oc5ccccc5c34)cc2)cc1] | 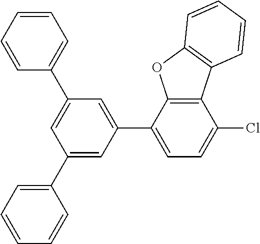 [SMILES: Clc1ccc(-c2cc(-c3ccccc3)cc(-c3ccccc3)c2)c2oc3ccccc3c12] | 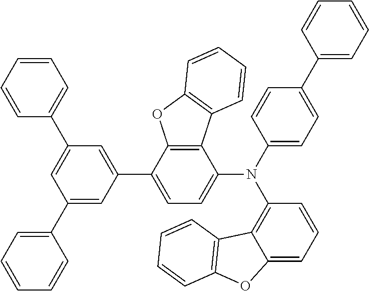 [SMILES: c1ccc(-c2ccc(N(c3cccc4oc5ccccc5c34)c3ccc(-c4cc(-c5ccccc5)cc(-c5ccccc5)c4)c4oc5ccccc5c34)cc2)cc1] | 20.7 g (81%) | 729.27 |
| 32 | 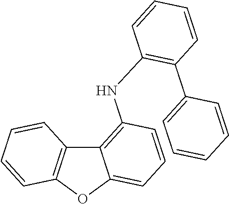 [SMILES: c1ccc(-c2ccccc2Nc2cccc3oc4ccccc4c23)cc1] | 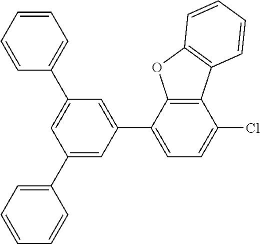 [SMILES: Clc1ccc(-c2cc(-c3ccccc3)cc(-c3ccccc3)c2)c2oc3ccccc3c12] | 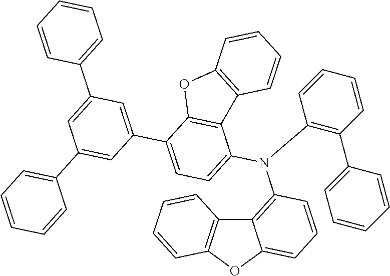 [SMILES: c1ccc(-c2cc(-c3ccccc3)cc(-c3ccc(N(c4ccccc4-c4ccccc4)c4cccc5oc6ccccc6c45)c4c3oc3ccccc34)c2)cc1] | 20.2 g (79%) | 729.27 |
| 9 | 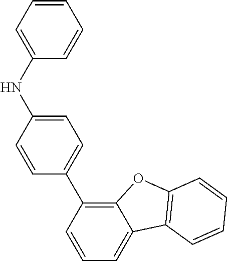 [SMILES: c1ccc(Nc2ccc(-c3cccc4c3oc3ccccc34)cc2)cc1] | 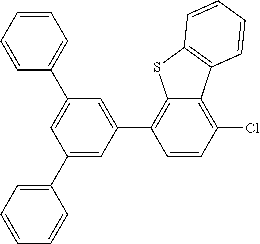 [SMILES: Clc1ccc(-c2cc(-c3ccccc3)cc(-c3ccccc3)c2)c2sc3ccccc3c12] | 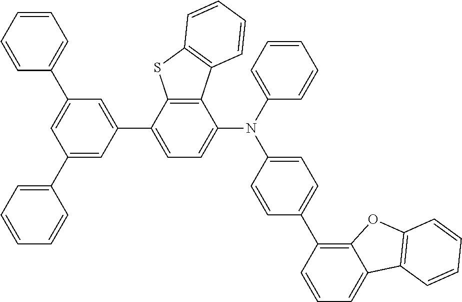 [SMILES: c1ccc(-c2cc(-c3ccccc3)cc(-c3ccc(N(c4ccccc4)c4ccc(-c5cccc6c5oc5ccccc56)cc4)c4c3sc3ccccc34)c2)cc1] | 21.1 g (81%) | 745.24 |
| 10 | 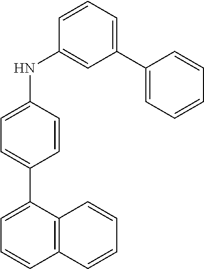 [SMILES: c1ccc(-c2cccc(Nc3ccc(-c4cccc5ccccc45)cc3)c2)cc1] | 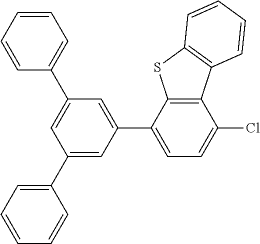 [SMILES: Clc1ccc(-c2cc(-c3ccccc3)cc(-c3ccccc3)c2)c2sc3ccccc3c12] | 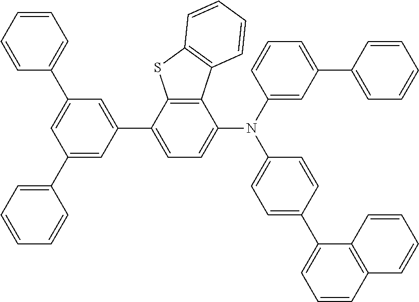 [SMILES: c1ccc(-c2cc(-c3ccccc3)cc(-c3ccc(N(c4ccc(-c5cccc6ccccc56)cc4)c4cccc(-c5ccccc5)c4)c4c3sc3ccccc34)c2)cc1] | 21.6 g (79%) | 781.28 |
| 125 | 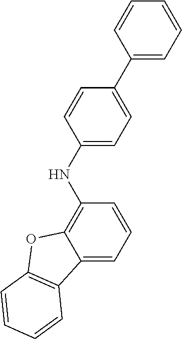 [SMILES: c1ccc(-c2ccc(Nc3cccc4c3oc3ccccc34)cc2)cc1] | 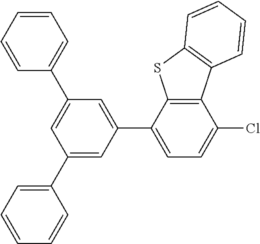 [SMILES: Clc1ccc(-c2cc(-c3ccccc3)cc(-c3ccccc3)c2)c2sc3ccccc3c12] | 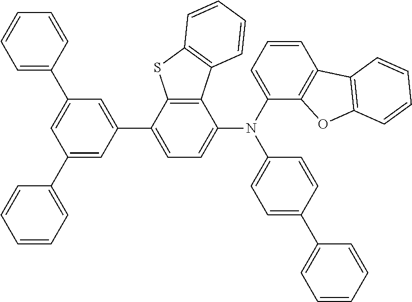 [SMILES: c1ccc(-c2ccc(N(c3cccc4c3oc3ccccc34)c3ccc(-c4cc(-c5ccccc5)cc(-c5ccccc5)c4)c4sc5ccccc5c34)cc2)cc1] | 21.4 g (82%) | 745.24 |
| 15 | 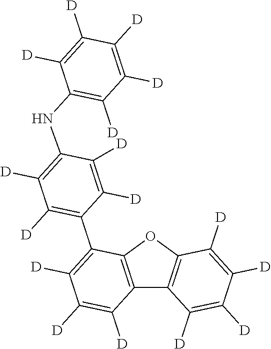 [SMILES: [2H]c1c([2H])c([2H])c(Nc2c([2H])c([2H])c(-c3c([2H])c([2H])c([2H])c4c3oc3c([2H])c([2H])c([2H])c([2H])c34)c([2H])c2[2H])c([2H])c1[2H]] | 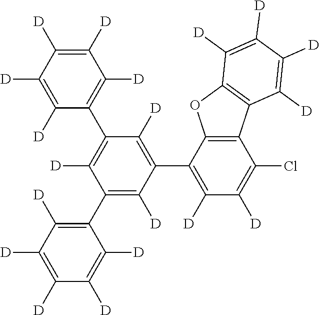 [SMILES: [2H]c1c([2H])c([2H])c(-c2c([2H])c(-c3c([2H])c([2H])c([2H])c([2H])c3[2H])c([2H])c(-c3c([2H])c([2H])c(Cl)c4c3oc3c([2H])c([2H])c([2H])c([2H])c34)c2[2H])c([2H])c1[2H]] | 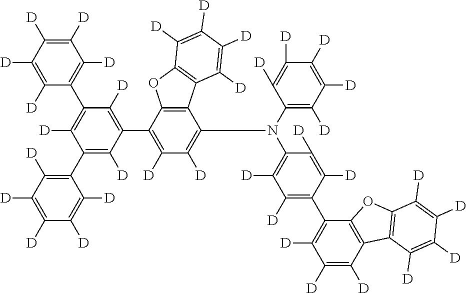 [SMILES: [2H]c1c([2H])c([2H])c(-c2c([2H])c(-c3c([2H])c([2H])c([2H])c([2H])c3[2H])c([2H])c(-c3c([2H])c([2H])c(N(c4c([2H])c([2H])c([2H])c([2H])c4[2H])c4c([2H])c([2H])c(-c5c([2H])c([2H])c([2H])c6c5oc5c([2H])c([2H])c([2H])c([2H])c56)c([2H])c4[2H])c4c3oc3c([2H])c([2H])c([2H])c([2H])c34)c2[2H])c([2H])c1[2H]] | 21.7 g (81%) | 764.49 |
| 206 | 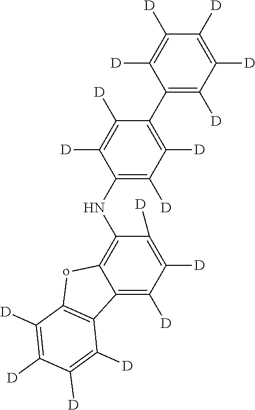 | 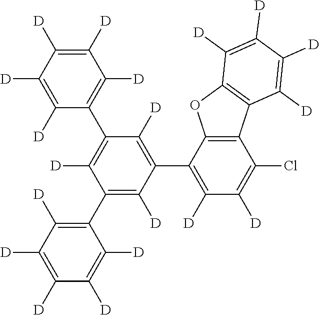 [SMILES: [2H]c1c([2H])c([2H])c(-c2c([2H])c(-c3c([2H])c([2H])c([2H])c([2H])c3[2H])c([2H])c(-c3c([2H])c([2H])c(Cl)c4c3oc3c([2H])c([2H])c([2H])c([2H])c34)c2[2H])c([2H])c1[2H]] | 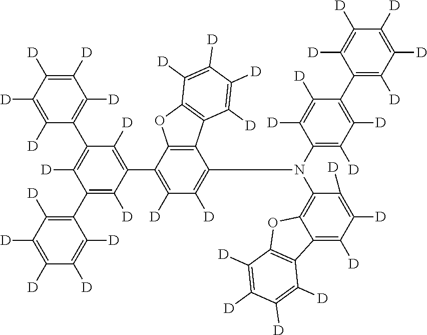 [SMILES: [2H]c1c([2H])c([2H])c(-c2c([2H])c([2H])c(N(c3c([2H])c([2H])c([2H])c4c3oc3c([2H])c([2H])c([2H])c([2H])c34)c3c([2H])c([2H])c(-c4c([2H])c(-c5c([2H])c([2H])c([2H])c([2H])c5[2H])c([2H])c(-c5c([2H])c([2H])c([2H])c([2H])c5[2H])c4[2H])c4oc5c([2H])c([2H])c([2H])c([2H])c5c34)c([2H])c2[2H])c([2H])c1[2H]] | 22.0 g (82%) | 764.49 |
| 16 | 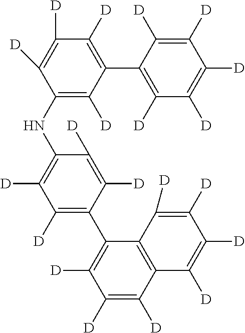 [SMILES: [2H]c1c([2H])c([2H])c(-c2c([2H])c([2H])c([2H])c(Nc3c([2H])c([2H])c(-c4c([2H])c([2H])c([2H])c5c([2H])c([2H])c([2H])c([2H])c45)c([2H])c3[2H])c2[2H])c([2H])c1[2H]] | 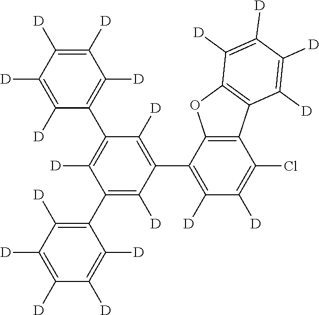 [SMILES: [2H]c1c([2H])c([2H])c(-c2c([2H])c(-c3c([2H])c([2H])c([2H])c([2H])c3[2H])c([2H])c(-c3c([2H])c([2H])c(Cl)c4c3oc3c([2H])c([2H])c([2H])c([2H])c34)c2[2H])c([2H])c1[2H]] | 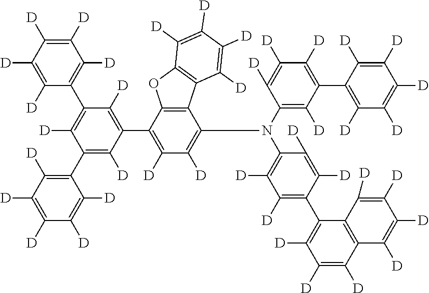 [SMILES: [2H]c1c([2H])c([2H])c(-c2c([2H])c(-c3c([2H])c([2H])c([2H])c([2H])c3[2H])c([2H])c(-c3c([2H])c([2H])c(N(c4c([2H])c([2H])c(-c5c([2H])c([2H])c([2H])c6c([2H])c([2H])c([2H])c([2H])c56)c([2H])c4[2H])c4c([2H])c([2H])c([2H])c(-c5c([2H])c([2H])c([2H])c([2H])c5[2H])c4[2H])c4c3oc3c([2H])c([2H])c([2H])c([2H])c34)c2[2H])c([2H])c1[2H]] | 35.7 g (79%) | 804.55 |
| 207 | 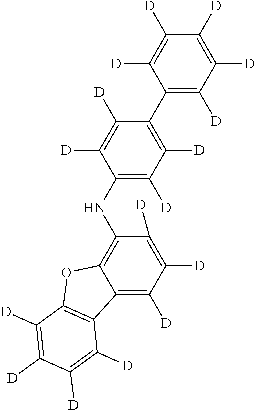 [SMILES: [2H]c1c([2H])c([2H])c(-c2c([2H])c([2H])c(Nc3c([2H])c([2H])c([2H])c4c3oc3c([2H])c([2H])c([2H])c([2H])c34)c([2H])c2[2H])c([2H])c1[2H]] | 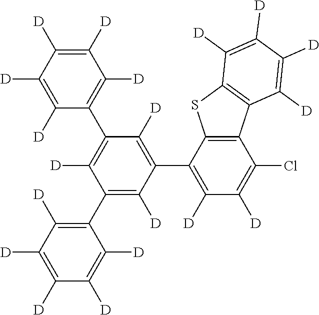 [SMILES: [2H]c1c([2H])c([2H])c(-c2c([2H])c(-c3c([2H])c([2H])c([2H])c([2H])c3[2H])c([2H])c(-c3c([2H])c([2H])c(Cl)c4c3sc3c([2H])c([2H])c([2H])c([2H])c34)c2[2H])c([2H])c1[2H]] | 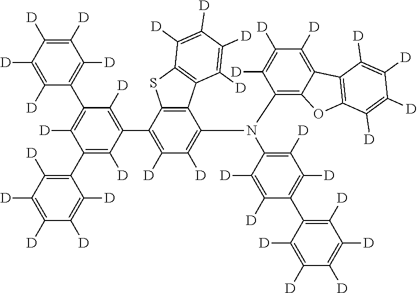 [SMILES: [2H]c1c([2H])c([2H])c(-c2c([2H])c([2H])c(N(c3c([2H])c([2H])c([2H])c4c3oc3c([2H])c([2H])c([2H])c([2H])c34)c3c([2H])c([2H])c(-c4c([2H])c(-c5c([2H])c([2H])c([2H])c([2H])c5[2H])c([2H])c(-c5c([2H])c([2H])c([2H])c([2H])c5[2H])c4[2H])c4sc5c([2H])c([2H])c([2H])c([2H])c5c34)c([2H])c2[2H])c([2H])c1[2H]] | 21.9 g (80%) | 780.46 |

Effects of the compound according to the present invention were confirmed through the following experiments, which are merely representative examples, and the experimental examples are not limited thereto.

### Experimental Example 1: Simulation Results for Hole Transport Auxiliary Layer

The hole transport auxiliary layer plays a role to reduce accumulation of holes at an interface of the emitting layer, caused by the HOMO level difference between the hole transport layer and the emitting layer. For this purpose, it is preferable that the HOMO energy difference with the emitting layer is smaller than the HOMO energy difference with the hole transport layer. Furthermore, in order to minimize migrating of electrons from the emitting layer due to a leakage into the hole transport layer, the hole transport auxiliary layer should have a LUMO energy level higher than that of the emitting layer.

To verify suitability of the compound represented by A of the present invention as a material for a hole transport auxiliary layer, a HOMO energy level (eV), and a LUMO energy level (eV) were calculated using Spartan software (B3LYP DFT 6-31G* by spartan '16), and the results are listed in Table 2 below.

TABLE 2

| Compounds | HOMO (calculation) | LUMO (calculation) |
|---|---|---|
| Compound 1 | −5.04 | −1.20 |
| Compound 2 | −5.04 | −1.19 |
| Compound 3 | −5.02 | −1.21 |
| Compound 4 | −5.07 | −1.19 |
| Compound 5 | −4.99 | −1.22 |
| Compound 6 | −4.98 | −1.11 |

### [Example 1] Manufacture of Organic Light-Emitting Diode (Blue Emitting Layer)

A substrate on which ITO (100 nm) was stacked, which serves as a first electrode (anode) of an organic light-emitting diode, was patterned into regions for a second electrode (cathode), the first electrode (anode), and an insulation layer through a photolithography process. Subsequently, a surface of the first electrode (ITO) was subjected to UV-ozone treatment and O₂:N₂ plasma treatment for purposes of increasing a work function thereof and cleaning.

Next, a mixture of 2-(7-dicyanomethylene-1,3,4,5,6,8,9,10-octafluoro-7H-pyren-2-ylidene)-malononitrile (NDP-9), and N4,N4,N4′,N4′-tetra([1,1′-biphenyl]-4-yl)-[1,1′-biphenyl]-4,4′-diamine at a ratio of approximately 3:97 was deposited on the anode as a hole injection layer (HIL) to a thickness of approximately 10 nm. Subsequently, N4,N4,N4′,N4′-tetra([1,1′-biphenyl]-4-yl)-[1,1′-biphenyl]-4,4′-diamine was vacuum deposited on the hole injection layer as a hole transport layer to a thickness of approximately 100 nm, and a hole transport auxiliary layer was formed using Compound 31 on the hole transport layer (HTL) to a thickness of approximately 15 nm.

9,10-bis(2-naphthyl) anthracene (ADN) as a host, and 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB) as a dopant were deposited on the hole transport auxiliary layer to form a blue emitting layer on the hole transport auxiliary layer to approximately 25 nm, where a mixing ratio (based on weight) of the host:dopant was approximately 97:3. A mixture of 2-(4-(9,10-di(naphthalene-2-yl) anthracene-2-yl)phenyl)-1-phenyl-1H-benzo[d]imidazole and Liq at a weight ratio of about 1:1 was deposited on the blue emitting layer to approximately 25 nm as an electron transport layer (ETL). On the electron transport layer (ETL), Liq was deposited as an electron injection layer to approximately 1 nm, and a mixture of magnesium and silver at a weight ratio of about 1:4 was deposited as a negative electrode to a thickness of approximately 16 nm. N4,N4′-bis[4-[bis(3-methyl phenyl)amino]phenyl]-N4,N4′-diphenyl-[1,1′-biphenyl]-4,4′-diamine (DNTPD) was deposited on the cathode as a capping layer to a thickness of approximately 60 nm. To protect the organic light-emitting diode from oxygen and moisture in an atmosphere, an encapsulation layer (or protecting layer) was formed by bonding a seal cap containing a desiccant with a UV-curable adhesive, thereby completing the manufacture of the organic light-emitting diode.

### Examples 2 to 57

Organic light-emitting diodes according to Examples 2 to 57 were manufactured in the same manner as the organic light-emitting diode according to Example 1, except that the material of Compound 31 used as the hole transport auxiliary layer material in Example 1 was changed to materials listed in Table 3 below.

### Comparative Examples 1 to 4

Organic light-emitting diodes according to Comparative Examples 1 to 4 were manufactured in the same manner as the organic light-emitting diode according to Example 1, except that the material of Compound 31 used as the hole transport auxiliary layer material in Example 1 was changed to Compound A, Compound B, Compound C, and Compound D below.

Compound A

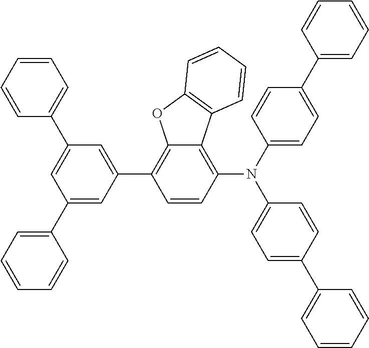 [SMILES: c1ccc(-c2ccc(N(c3ccc(-c4ccccc4)cc3)c3ccc(-c4cc(-c5ccccc5)cc(-c5ccccc5)c4)c4oc5ccccc5c34)cc2)cc1]

Compound B

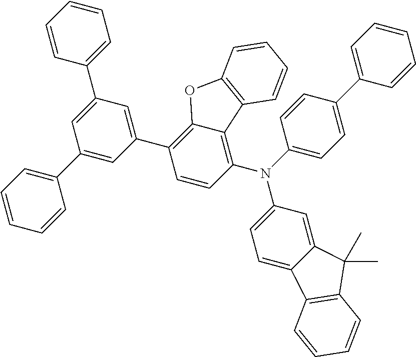 [SMILES: CC1(C)c2ccccc2-c2ccc(N(c3ccc(-c4ccccc4)cc3)c3ccc(-c4cc(-c5ccccc5)cc(-c5ccccc5)c4)c4oc5ccccc5c34)cc21]

Compound C

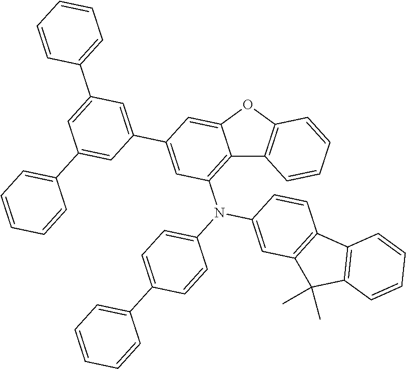 [SMILES: CC1(C)c2ccccc2-c2ccc(N(c3ccc(-c4ccccc4)cc3)c3cc(-c4cc(-c5ccccc5)cc(-c5ccccc5)c4)cc4oc5ccccc5c34)cc21]

Compound D

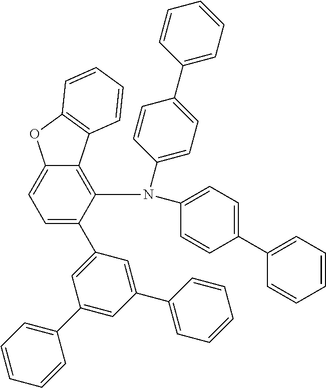 [SMILES: c1ccc(-c2ccc(N(c3ccc(-c4ccccc4)cc3)c3c(-c4cc(-c5ccccc5)cc(-c5ccccc5)c4)ccc4oc5ccccc5c34)cc2)cc1]

### [Experimental Example 2] Organic Light-Emitting Diode Performance Evaluation (Blue Element)

For each of the organic light-emitting diodes manufactured in Examples 1 to 57 and Comparative Examples 1 to 4, a driving voltage (V), efficiency (Cd/A), and Y color coordinate (based on CIE 1931, CIEy) were measured by applying a current of 10 mA/cm² using a Konica Minolta CS-2000 spectroradiometer. In addition, a lifetime (LT95, hrs) was measured using a McScience M6000 under a constant current driving of 10 mA/cm² by checking the time taken for luminance to decrease to 95% from 100% of an initial luminance. In addition, Blue Index (BI=(Cd/A)/CIEy) values were calculated using efficiency and color coordinate values and recorded accordingly. The evaluation results are listed in Table 3 below.

TABLE 3

| | | | | | | Life- |
|---|---|---|---|---|---|---|
| Example/ | Hole transport | Driving | Effi- | | | time |
| Comparative | auxiliary layer | voltage | ciency | | | (LT95) |
| Example | compound | (V) | (Cd/A) | CIEy | BI | (hrs) |
| Example 1 | 25 | 3.42 | 8.0 | 0.044 | 181.8 | 456 |
| Example 2 | 5 | 3.39 | 7.9 | 0.045 | 175.6 | 454 |
| Example 3 | 26 | 3.40 | 7.8 | 0.045 | 173.3 | 452 |
| Example 4 | 27 | 3.41 | 8.1 | 0.043 | 188.4 | 458 |
| Example 5 | 28 | 3.34 | 8.4 | 0.044 | 190.9 | 464 |
| Example 6 | 29 | 3.31 | 8.3 | 0.045 | 184.4 | 462 |
| Example 7 | 30 | 3.32 | 8.2 | 0.044 | 186.4 | 460 |
| Example 8 | 31 | 3.33 | 8.5 | 0.043 | 197.7 | 466 |
| Example 9 | 32 | 3.38 | 7.6 | 0.043 | 176.7 | 445 |
| Example 10 | 6 | 3.35 | 7.4 | 0.044 | 168.2 | 447 |
| Example 11 | 33 | 3.36 | 7.5 | 0.044 | 170.5 | 445 |
| Example 12 | 34 | 3.37 | 7.7 | 0.044 | 175.0 | 449 |
| Example 13 | 35 | 3.41 | 8.0 | 0.044 | 181.8 | 456 |
| Example 14 | 36 | 3.38 | 7.9 | 0.045 | 175.6 | 454 |
| Example 15 | 3 | 3.40 | 8.1 | 0.043 | 188.4 | 458 |
| Example 16 | 38 | 3.43 | 7.5 | 0.044 | 170.5 | 449 |
| Example 17 | 41 | 3.44 | 7.6 | 0.045 | 168.9 | 452 |
| Example 18 | 42 | 3.38 | 7.8 | 0.044 | 177.3 | 445 |
| Example 19 | 43 | 3.35 | 7.7 | 0.045 | 171.1 | 443 |
| Example 20 | 44 | 3.36 | 7.7 | 0.045 | 171.1 | 441 |
| Example 21 | 45 | 3.37 | 7.9 | 0.043 | 183.7 | 447 |
| Example 22 | 48 | 3.41 | 7.7 | 0.044 | 175.0 | 441 |
| Example 23 | 49 | 3.42 | 7.5 | 0.045 | 166.7 | 437 |
| Example 24 | 51 | 3.37 | 7.8 | 0.045 | 173.3 | 444 |
| Example 25 | 52 | 3.34 | 7.6 | 0.044 | 172.7 | 442 |
| Example 26 | 53 | 3.35 | 7.7 | 0.045 | 171.1 | 440 |
| Example 27 | 54 | 3.36 | 7.8 | 0.043 | 181.4 | 446 |
| Example 28 | 57 | 3.40 | 7.6 | 0.045 | 168.9 | 440 |
| Example 29 | 58 | 3.41 | 7.5 | 0.044 | 170.5 | 436 |
| Example 30 | 60 | 3.52 | 8.0 | 0.044 | 181.8 | 449 |
| Example 31 | 63 | 3.61 | 8.0 | 0.043 | 186.0 | 452 |
| Example 32 | 64 | 3.54 | 8.3 | 0.045 | 184.4 | 456 |
| Example 33 | 71 | 3.57 | 7.8 | 0.043 | 181.4 | 443 |
| Example 34 | 75 | 3.60 | 8.0 | 0.043 | 186.0 | 452 |
| Example 35 | 82 | 3.55 | 7.7 | 0.045 | 171.1 | 435 |
| Example 36 | 90 | 3.58 | 7.8 | 0.044 | 177.3 | 437 |
| Example 37 | 1 | 3.38 | 7.5 | 0.045 | 166.7 | 416 |
| Example 38 | 98 | 3.41 | 7.5 | 0.045 | 166.7 | 415 |
| Example 39 | 99 | 3.36 | 7.4 | 0.045 | 164.4 | 412 |
| Example 40 | 100 | 3.39 | 7.3 | 0.045 | 162.2 | 411 |
| Example 41 | 101 | 3.37 | 7.2 | 0.045 | 160.0 | 410 |
| Example 42 | 4 | 3.32 | 7.6 | 0.045 | 168.9 | 418 |
| Example 43 | 103 | 3.35 | 7.5 | 0.045 | 166.7 | 417 |
| Example 44 | 104 | 3.31 | 7.4 | 0.045 | 164.4 | 409 |
| Example 45 | 106 | 3.34 | 7.3 | 0.045 | 162.2 | 412 |
| Example 46 | 107 | 3.37 | 7.3 | 0.045 | 162.2 | 411 |
| Example 47 | 108 | 3.45 | 7.5 | 0.045 | 166.7 | 415 |
| Example 48 | 2 | 3.43 | 7.4 | 0.045 | 164.4 | 416 |
| Example 49 | 111 | 3.42 | 7.2 | 0.045 | 160.0 | 412 |
| Example 50 | 112 | 3.46 | 7.4 | 0.045 | 164.4 | 415 |
| Example 51 | 9 | 3.60 | 7.3 | 0.044 | 165.9 | 402 |
| Example 52 | 10 | 3.52 | 7.2 | 0.045 | 160.0 | 398 |
| Example 53 | 126 | 3.61 | 7.2 | 0.044 | 163.6 | 406 |
| Example 54 | 134 | 3.61 | 7.3 | 0.045 | 162.2 | 400 |
| Example 55 | 15 | 3.40 | 8.1 | 0.043 | 188.4 | 520 |
| Example 56 | 206 | 3.42 | 8.1 | 0.044 | 184.1 | 532 |
| Example 57 | 16 | 3.32 | 7.6 | 0.045 | 168.9 | 504 |
| Comparative | Compound A | 3.74 | 6.4 | 0.045 | 142.2 | 371 |
| Example 1 | | | | | | |
| Comparative | Compound B | 3.70 | 6.2 | 0.045 | 137.8 | 375 |
| Example 2 | | | | | | |
| Comparative | Compound C | 3.70 | 6.1 | 0.045 | 135.6 | 362 |
| Example 3 | | | | | | |
| Comparative | Compound D | 3.78 | 5.7 | 0.045 | 126.7 | 368 |
| Example 4 | | | | | | |

In the organic compound represented by Chemical Formula A of the present invention, umbrella-shaped terphenyl is substituted at a 4-position of dibenzofuran an arylamine group is included at a 1-position, and a heteroaryl group or a fused polycyclic group is included at Ar. From Table 3, it was confirmed that organic light-emitting diodes including the organic compound represented by Chemical Formula A of the present invention may achieve excellent color realization while ensuring low driving voltage, high current efficiency, and long lifetime characteristics.

On the contrary, in Comparative Example 1 and 2, there is no heteroaryl group or a fused polycyclic aryl group, and Comparative Example 3 and 4 differ from the present invention in substitution positions a terphenyl and an amine group substituted for dibenzofuran. As a result of element evaluation, it can be confirmed that Comparative Example 1, 2, 3, and 4 exhibit poor element performance compared to the compound represented by Chemical Formula A of the present invention.

### [Experimental Example 3] Measurement of Lowest Triplet Energy (T₁)

For the compound represented by Chemical Formula A of the present invention and Comparative Example Compounds A, B, and C, a lowest triplet energy (Triplet 1, referred to as “T₁”) (unit: eV, absolute value) was calculated through quantum calculation experiments using Gaussian software (B3LYP DFT 6-31G* by Gaussian 16), and the results are shown in Table 4 below.

TABLE 4

| Compound | T₁(calculation) (eV) |
|---|---|
| Compound 3 | 2.71 |
| Compound 5 | 2.71 |
| Compound 6 | 2.72 |
| Compound 27 | 2.72 |
| Compound 35 | 2.73 |
| Compound 9 | 2.77 |
| Compound 126 | 2.81 |
| Compound A | 2.62 |
| Compound B | 2.58 |
| Compound C | 2.61 |

As confirmed in Table 4, the organic compound represented by Chemical Formula A of the present invention has a triplet energy level (T₁) in an appropriate range for a hole transport layer or a hole transport auxiliary layer. Therefore, the diffusion of triplet excitons generated in the emitting layer to the outside may be effectively suppressed, and the light-emitting efficiency and element lifetime characteristics may be improved.

Meanwhile, when the triplet energy level is low, the diffusion of triplet excitons generated in the emitting layer may not be prevented, and thus the efficiency of the element may decrease.

Accordingly, exemplary embodiments disclosed in the present specification are not intended to limit the technical idea of the present specification but to describe the technical idea of the present specification, and the scope of the technical idea of the present specification is not limited by such exemplary embodiments. Therefore, it should be understood that the examples described above should be understood as exemplary and be not limited thereto.

What is claimed is:

1. An organic compound represented by Chemical Formula A below: 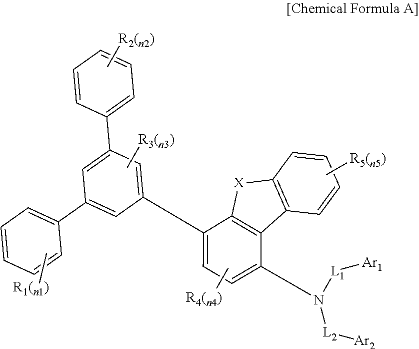
wherein, in Chemical Formula A above,
X is oxygen (O), or sulfur(S),
n1 and n2 are each an integer of 0 to 5,
n3 is an integer of 0 to 3,
n4 is an integer of 0 to 2,
n5 is an integer of 0 to 4,
L₁ and L₂ are the same as, or different from each other and are each independently a single bond, or a substituted or unsubstituted arylene group having 6 to 30 carbon atoms,
Ar₁ and Ar₂ are the same as, or different from each other and are each independently a substituted or unsubstituted aryl group having 6 to 30 carbon atoms, or a substituted or unsubstituted heteroaryl group having 3 to 60 carbon atoms,
R₁ to R₅ are the same as, or different from each other and are each independently hydrogen, deuterium, a substituted or unsubstituted alkyl group having 1 to 10 carbon atoms, a substituted or unsubstituted aryl group having 6 to 30 carbon atoms, or a substituted or unsubstituted heteroaryl group having 3 to 60 carbon atoms, and
when L₁, L₂, Ar₁, Ar₂, and R₁ to R₅ are each substituted, the substituents may be one or more selected from the group consisting of deuterium, a cyano group, a nitro group, a halogen group, a hydroxyl group, an alkyl group having 1 to 10 carbon atoms, a cycloalkyl group having 3 to 10 carbon atoms, an aryl group having 6 to 30 carbon atoms, a heteroaryl group having 3 to 60 carbon atoms, an alkylamino group having 1 to 10 carbon atoms, an arylamino group having 6 to 30 carbon atoms, a heteroarylamino group having a 1 to 30 carbon atoms, an alkylsilyl group having a 1 to 10 carbon atoms, an arylsilyl group having a 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms, and if there are a plurality of substituents, the substituents are the same as or different from each other.

2. The organic compound of claim 1, wherein Ar₁ is selected from the group consisting of substituents represented by M1 to M44 below: 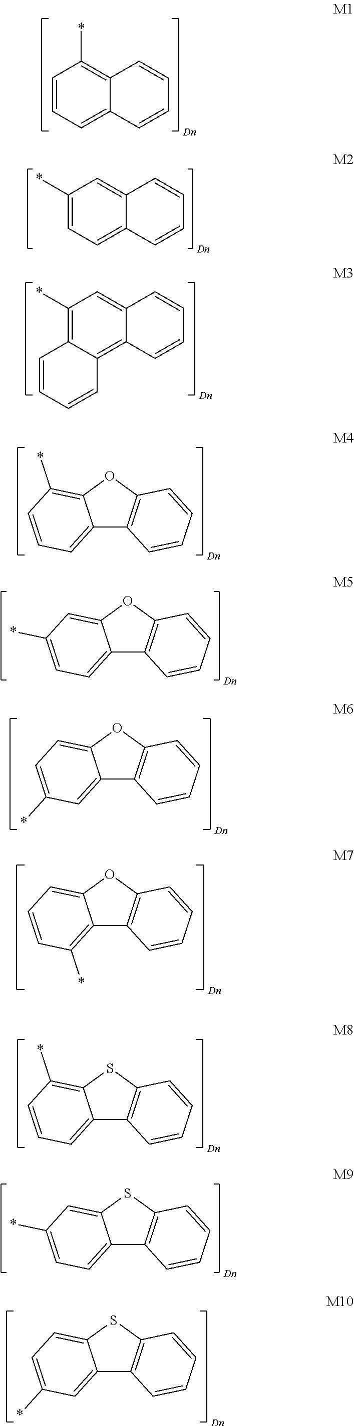 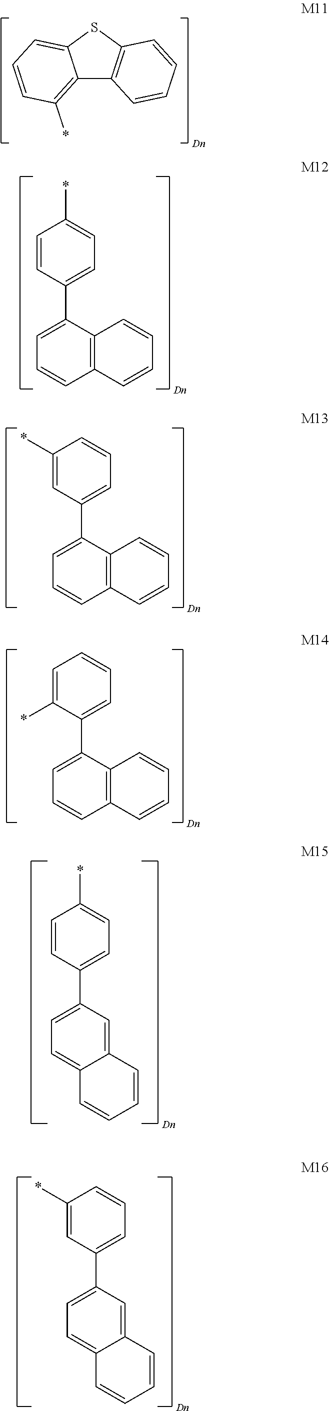 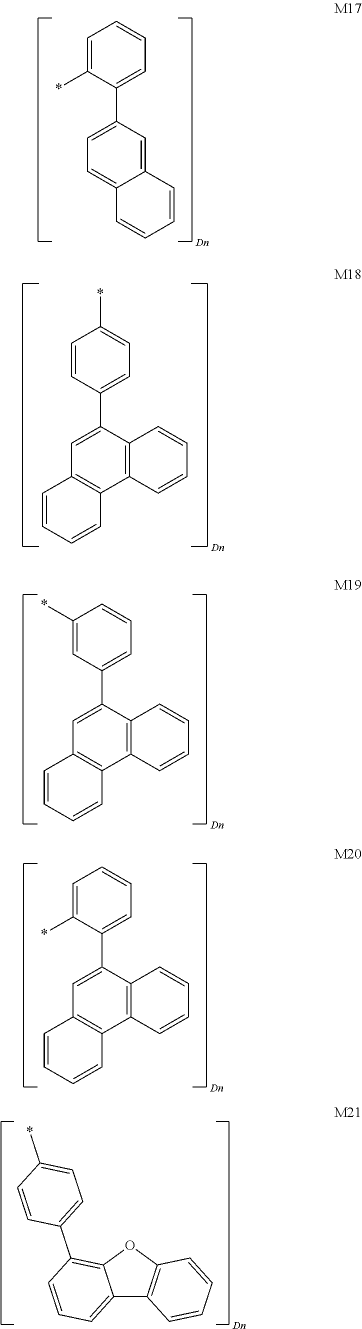 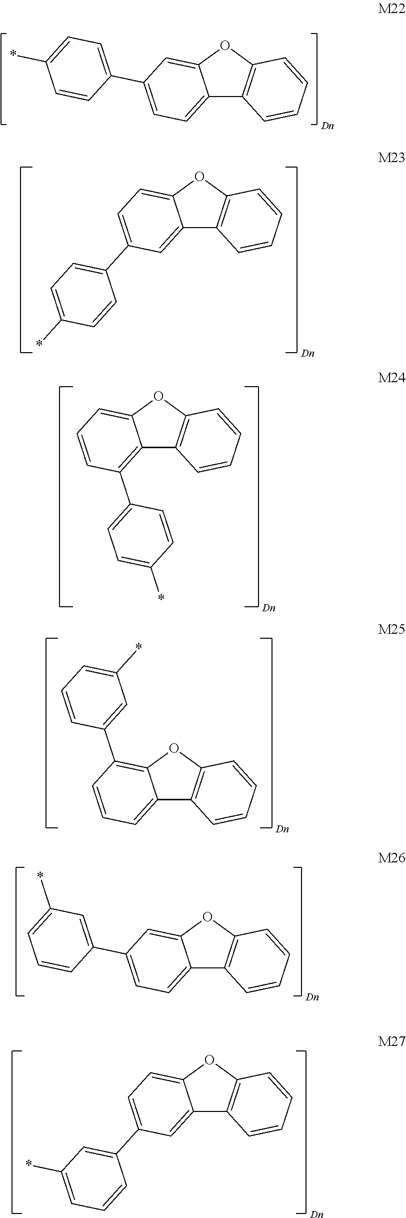 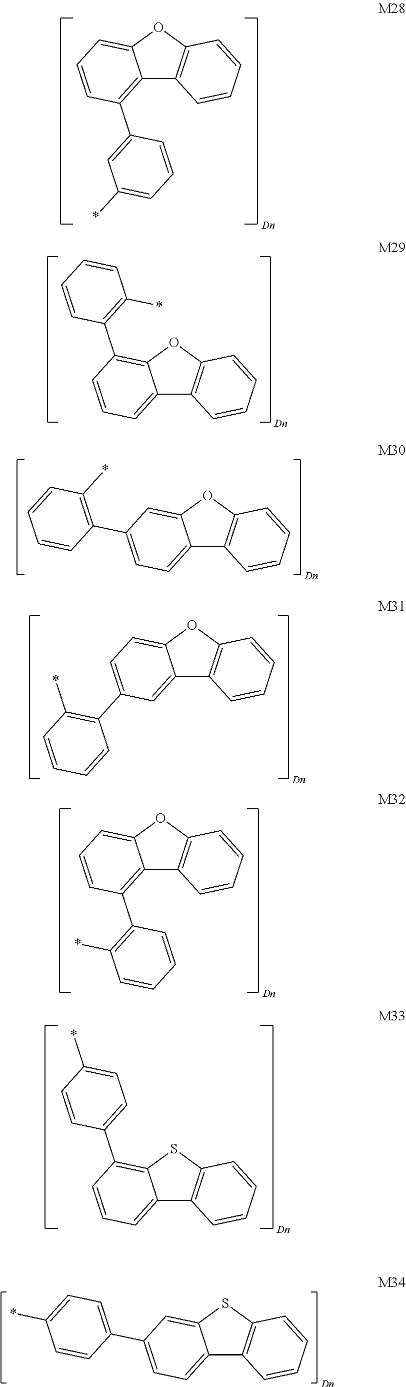 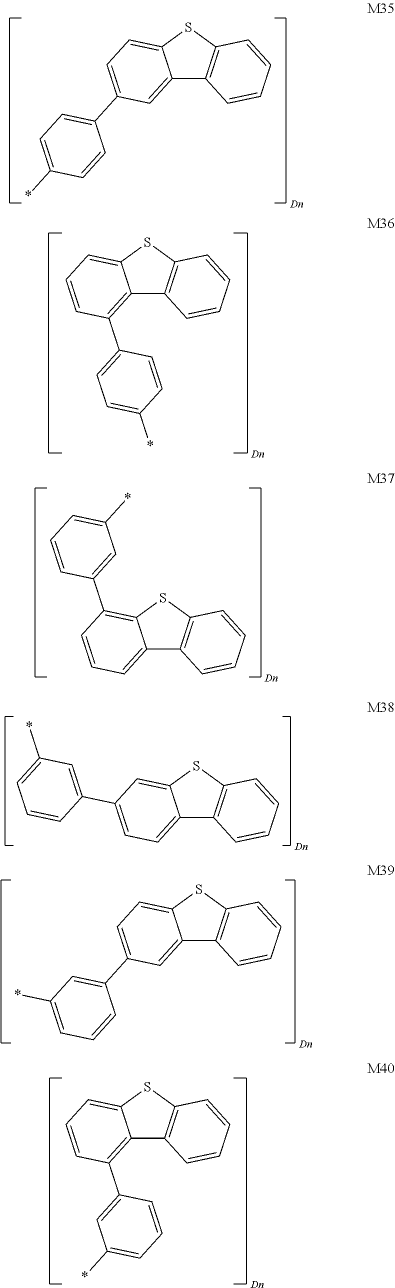 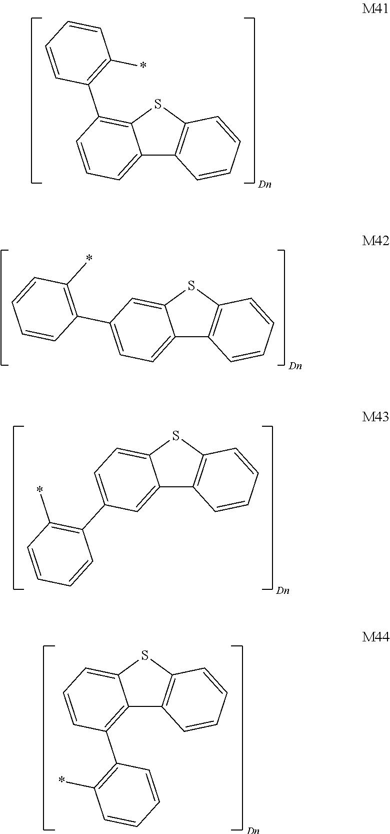
where, * indicates a bonding position.

3. The organic compound of claim 1, wherein Ar₂ is selected from the group consisting of substituents represented by A1 to A58 below: 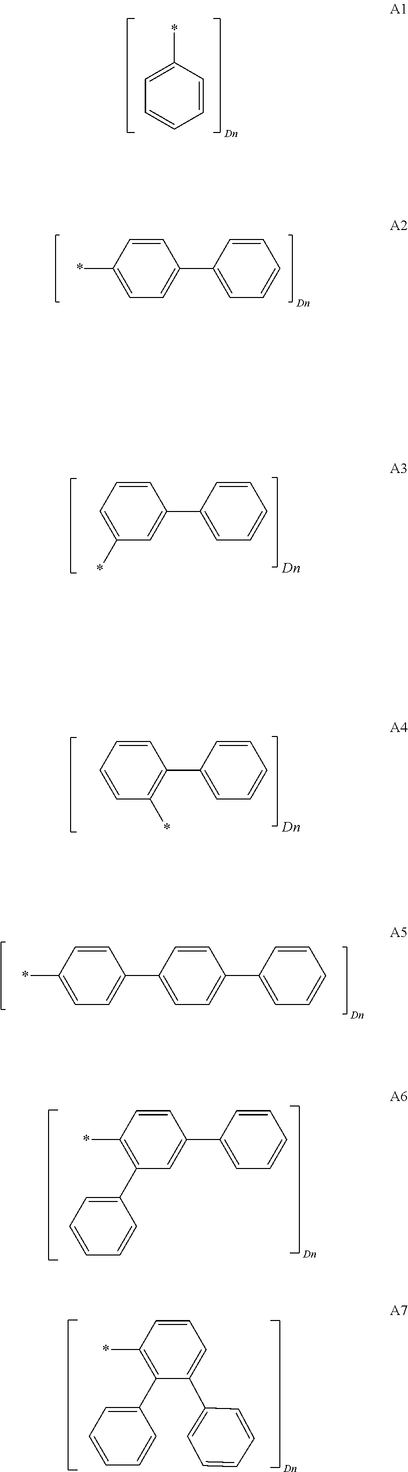 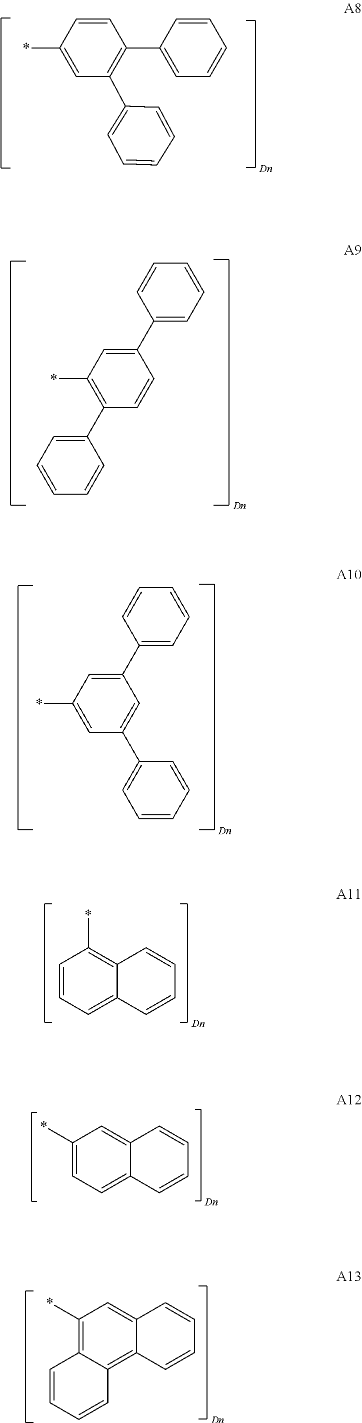 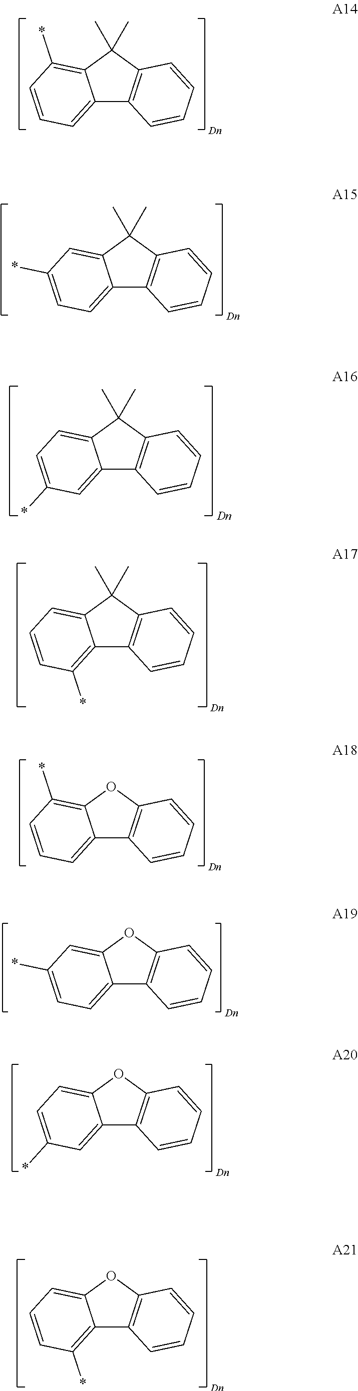 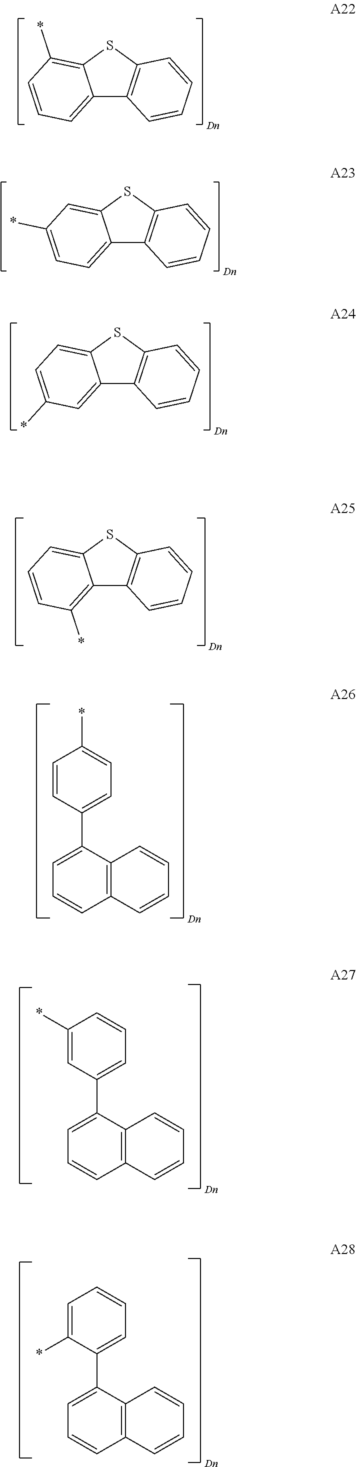 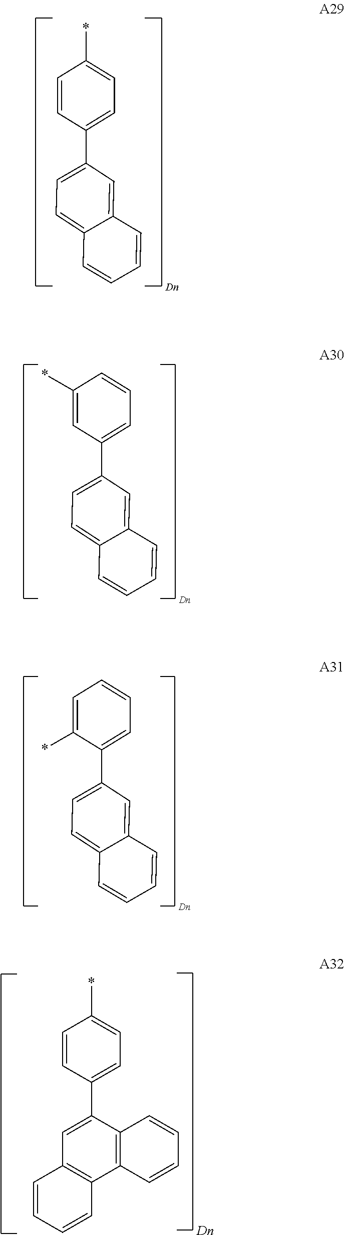 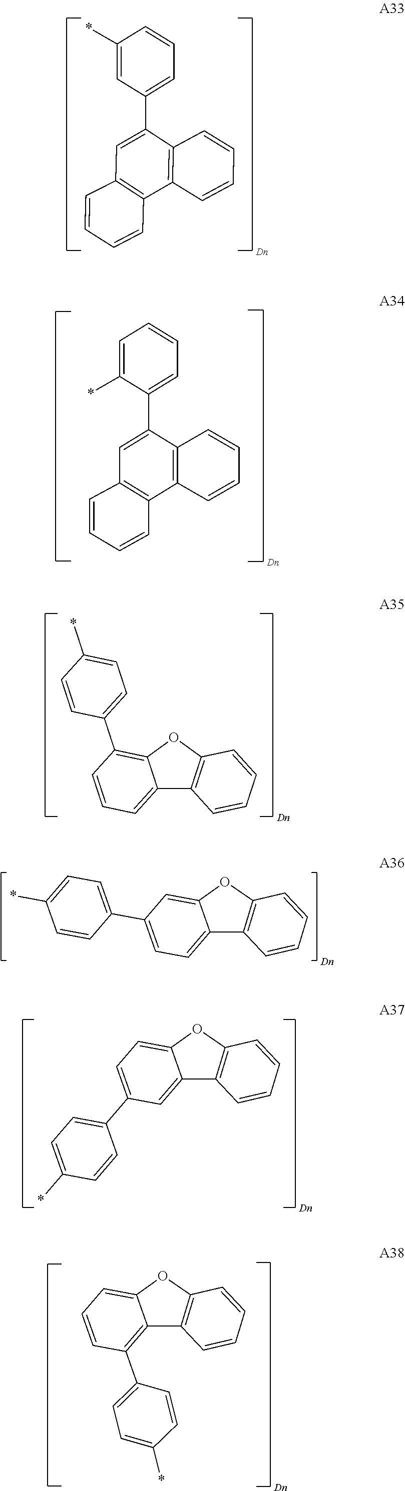 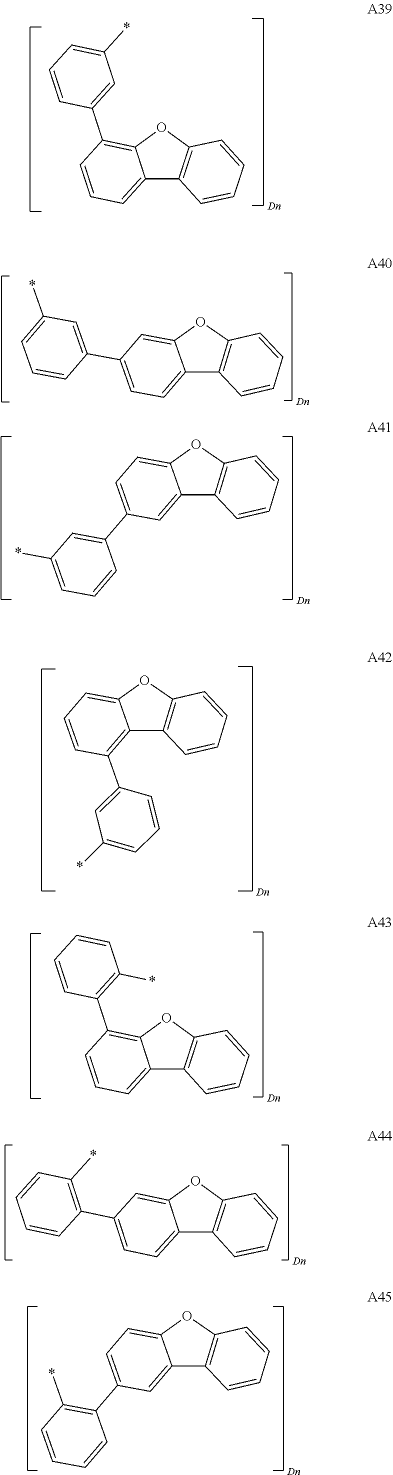 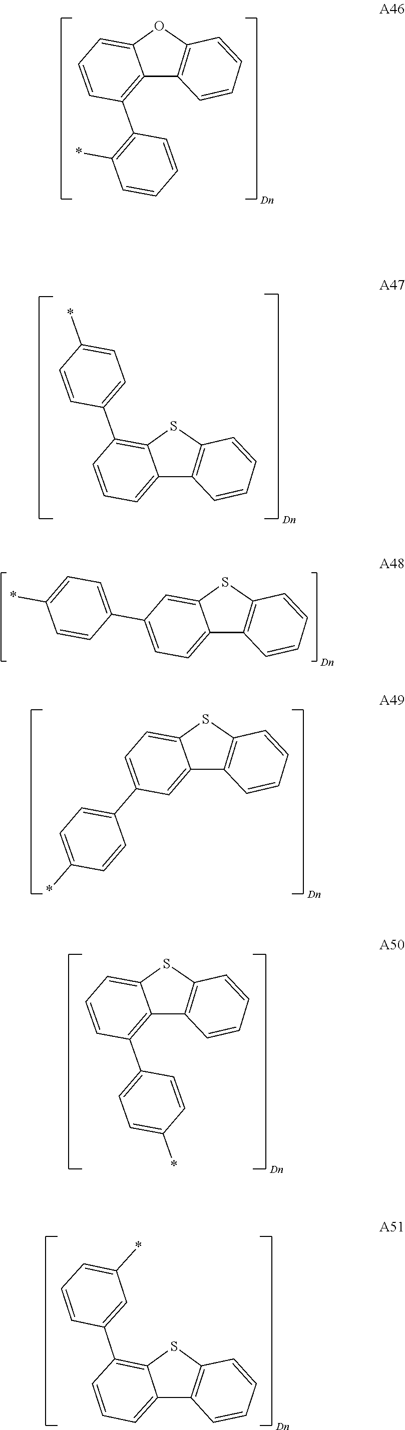 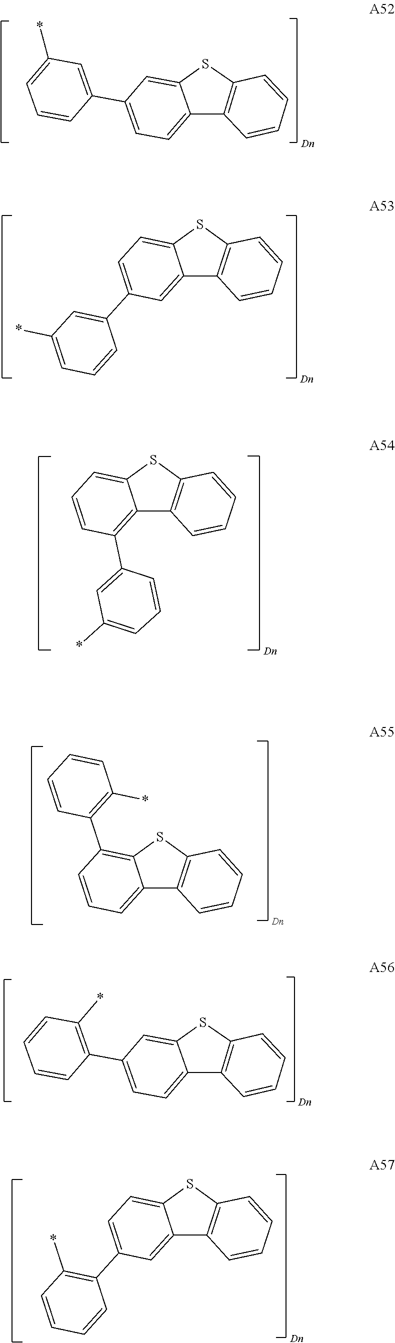 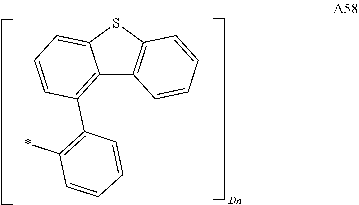
where, * indicates a bonding position.

4. An organic light-emitting diode comprising:
a first electrode;
a second electrode facing the first electrode, and
one or more organic material layers disposed between the first electrode and the second electrode,
wherein at least one among one or more organic material layers comprises an organic compound represented by Chemical Formula A of claim 1.

5. The organic light-emitting diode of claim 4, wherein the organic material layer comprising the organic compound represented by Chemical Formula A is a hole transport layer or a hole transport auxiliary layer.

6. A display device comprising the organic light-emitting diode of claim 4.
